(19) United States
(12) Patent Application Publication
(10) Pub. No.: US 2014/0057953 A1
(43) Pub. Date: Feb. 27, 2014

(54) BIARYL DERIVATIVES AS SELECTIVE 17BETA-HYDROXYSTEROID DEHYDROGENASE TYPE 2 INHIBITORS

(71) Applicants: Rolf Hartmann, Saarbrucken (DE); Sandrine Marchais-Oberwinkler, Blieskastel (DE); Kuiying Xu, Saarbrucken (DE); Ruth Werth, Dudweiler (DE)

(72) Inventors: Rolf Hartmann, Saarbrucken (DE); Sandrine Marchais-Oberwinkler, Blieskastel (DE); Kuiying Xu, Saarbrucken (DE); Ruth Werth, Dudweiler (DE)

(21) Appl. No.: 14/002,784

(22) PCT Filed: Mar. 2, 2012
(86) PCT No.: PCT/EP2012/053653

§ 371 Date: Nov. 13, 2013

(30) Foreign Application Priority Data
Mar. 3, 2011 (EP) 11156733.5

(51) Int. Cl. C07D 333/38; C07D 333/34; C07D 277/56; C07C 235/42; C07C 233/75

(52) U.S. Cl. CPC C07D 333/38; C07C 235/42; C07C 233/75; C07D 277/56; C07D 333/34
USPC 514/365; 564/176; 514/622; 564/161; 514/617; 549/72; 514/448; 548/200; 549/65; 514/445

(57) ABSTRACT

The invention relates to selective, non-steroidal 17beta-hydroxysteroid dehydrogenase type 2 (l7beta-HSD2) inhibitors of formula (I), their production and use, notably for the treatment and prophylaxis of sex steroid deficient diseases like osteoporosis in men and women.

The invention relates to selective, non-steroidal 17beta-hydroxysteroid dehydrogenase type 2 (17β-HSD2) inhibitors their production and use, notably for the treatment and prophylaxis of sex steroid deficient diseases like osteoporosis in men and women.

### BACKGROUND OF THE INVENTION

Estrogens and androgens play a crucial role in the development, growth and function of all tissues involved in reproduction and fertility. It is also well known, that estradiol (E2) and testosterone/dihydrotestosterone (T/DHT) the most active estrogen and androgen, respectively, can be involved in a series of hormone-sensitive diseases. For example estrogens or androgens are often responsible for the development of breast cancer or prostate cancer respectively, via stimulation of cell proliferation in the corresponding tissues (Travis, R. C. et al., Breast Cancer Res., 5:239-247 (2003); Wilding, G., Cancer Surv., 14:113-130 (1992)) or insufficient levels of E2 and T/DHT predispose the human skeleton to osteoporosis in both men and women (Pietschmann, P. et al., Gerontology, 55:3-12 (2009)). Osteoporosis is a systemic skeletal disease characterized by deterioration of bone tissue and low bone mass, resulting in increased fragility of the bone and higher risk of fractures of the hip, spine and wrist. Osteoporotic fractures lead to pain, occasional disability and more important, they increase mortality (Cree, M. et al., J. Am. Geriatr. Soc., 48:283-288 (2000)).

In healthy individuals, bone mass is maintained by a balance between bone resorption and bone formation performed by the osteoclasts (OCs) and osteoblasts (OBs), respectively. This process of bone remodelling facilitates repair of microdamage, provides calcium uptake and therefore brings stability and strength to the bone.

Bone loss is, however, accelerated in post-menopausal women and in elderly men. The mechanisms by which elderly people, both men and women, lose bone are not fully understood and remain under investigation. Decreased quantity of sex hormones is one important factor causing bone loss. In women at menopause, estrogen deficiency (Cree, M. et al., J. Am. Geriatr. Soc., 48:283-288 (2000)) and in older men, estrogen and androgen insufficiency (Fink, H. A. et al., J. Clin. Endocrinol. Metab., 91:3908-3915 (2006); Meier, C. et al., Arch. Intern. Med., 168:47-54 (2008)) result in a disproportionate increase in bone loss as compared with bone formation and often lead to osteoporosis.

Since OBs and OCs express estrogen receptors (Hoyland, J. A. et al., Bone, 20:87-92 (1997)) and respond to estrogen treatments, the most potent estrogen, E2 must have a direct effect on maintenance of bone mineral density. There are also substantial evidence that androgens like testosterone (T) and dihydrotestosterone (DHT) may as well be involved in bone formation, increasing OB activity and therefore protecting the bones against osteoporosis (Vanderschueren, D. et al., Endocr. Rev., 25:389-425 (2004), Vanderschueren, D. et al., Curr. Opin. Endocrinol. Diabetes Obes., 15:250-254 (2008)). T and DHT might act via activation of the androgen receptor (AR) which is present in the OBs (Bland, R., Clin. Sci., 98:217-240 (2000)) or could be the precursor of estrogens (the enzyme aromatase, responsible for the transformation of androgens in estrogens has been identified in the OBs). Controlled increase of E2 and T in bones of osteoporotic patients will simultaneously lower bone resorption (effect of E2) and raise bone formation (effect of T) improving bone loss and osteoporotic fractures. Augmentation of E2 and T levels in bones might be achieved by inhibition of the enzyme 17β-hydroxysteroid dehydrogenase type 2 (17β-HSD2) which has been identified in OBs (Dong, Y. et al., J. Bone Miner. Res., 13:1539-1546 (1998); Feix, M. et al., Mol. Cell. Endocrinol., 171:163-164 (2001); Janssen, J: M: et al., J. Cell. Biochem., 75:528-37 (1999)). A restricted increase in E2 and T in OCs and OBs is important to avoid unwanted side-effects such as induction of breast cancer or prostate cancer. This might be achieved via an intracrine mechanism (Labrie, F., Mol. Cell. Endocrinol. 78:C113-118 (1991)), i.e. the transformation of E2 and T in inactive precursors should be blocked dominantly in the bone cells.

17β-HSD2 (Wu, L. et al., J. Biol. Chem., 268:12964-12969 (1993); Lu, M., J. Biol. Chem., 277:22123-22130 (2002)) belongs to the hydroxysteroid dehydrogenase (HSD) superfamily. The HSD enzymes play pivotal roles not only in the activation but also in the inactivation of steroid hormones. Depending on the need of the cell, they modulate the potency of the sex hormones (Penning, T. M. et al., Endocr. Rev., 18:281-305 (1997)). For example, 17β-HSD1 activates E1 into the very potent E2, while 17β-HSD2 oxidizes E2 into E1 thus decreasing the action of the potent E2. It is therefore believed that in physiology 17β-HSD2 protects the cell against excessive level of active estrogen.

To date, fourteen different 17β-HSDs have been identified (Lukacik, P. et al., Mol. Cell. Endocrinol. 248:61-71 (2006); Luu-The, V. et al., Best Pract. Res. Clin. Endocrinol. Metab., 22:207-221 (2008)) and twelve different subtypes have been cloned from human tissues. They all belong to the Short-Chain Dehydrogenase/Reductase family (except type 5). The 17β-HSDs show little amino acid identity (20-30%) and are membrane-bound or soluble enzymes. The X-ray structures of six human subtypes (type 1, 4, 8, 10, 11, 14) are known (Moeller, G. et al., Mol. Cell. Endocrinol. 301:7-19 (2009)). All of them are NAD(H)- or NADP(H)-dependent. The 17β-HSDs play therefore a key role in hormonal regulation and function in humans. They differ in tissue distribution, catalytic preference (oxidation or reduction), substrate specificity and sub-cellular localisation. It is likely that all 17β-HSDs, modulating estrogen and androgen action, are expressed in the different estrogen/androgen-dependent tissues but certainly at different concentrations. In diseased tissues, the ratio between the different subtypes is changed compared to healthy tissues: one enzyme subtype might be overexpressed or completely absent, leading to higher/lower concentrations of the specific steroids. Selective inhibition of one subtype could therefore be a good strategy to influence the level in estrogen and androgen in hormone sensitive diseases.

17β-HSD2 (EC1.1.1.51) (Wu, L. et al., J. Biol. Chem., 268:12964-12969 (1993); Lu, M., J. Biol. Chem., 277:22123-22130 (2002)) is a trans-membrane protein localized in the endoplasmic reticulum. Its 3D-structure remains unknown. 178-HSD2 shows a broad tissue distribution in adult: it is expressed in liver, small intestine, endometrium, urinary tracts and bone osteoblastic (Dong, Y. et al., J. Bone Miner. Res., 13:1539-1546 (1998); Feix, M. et al., Mol. Cell. Endocrinol., 171:163-164 (2001); Eyre, L. J. et al., J. Bone Miner. Res., 13:996-1004 (1998)) and osteoclastic (van der Eerden, B. C. J. et al., J. Endocrinol., 180:457-467 (2004)) cells. It has a predominant oxidative activity on hydroxy groups at the position C17 of the steroids: it converts the highly active 17β-hydroxysteroid estrogen E2 as well as the 17β-hydroxysteroid androgens T and DHT into their inactive keto forms using the cofactor NAD⁺. The broad tissue distribution together with the oxidative activity of 17β-HSD2 suggests that this enzyme may play an essential role in the inactivation of highly active estrogens and androgens, resulting in diminished sex hormone action in target tissues.

Only very few inhibitors of 17β-HSD2 have been described: Poirier et al. (Sam, K. M., 3. Med. Chem., 38:4518-4528 (1995); Poirier, D. et al., Mol. Cell. Endocrinol., 171:119-128 (2001); Bydal, P. et al., Eur. J. Med. Chem., 44:632-644 (2009)) reported on the potent steroidal C17-spirolactone 1 (inhibitory activity of 70% at 1 μM and 40% at 100 nM). Cook et al. (Cook, J. H. et al., Tetrahedron Letters, 46:1525-1528 (2005); Gunn, D., Bioorg. Med. Chem. Lett., 15:3053-3057 (2005); Wood, J. et al., Bioorg. Med. Chem. Lett., 16:4965-4968 (2006)) found, using high through-put screening methods, the first new class of potent non-steroidal inhibitors of 17β-HSD2: the 4,5-disubstituted cis-pyrrolidines. The most active compound described is 2 (IC₅₀=10 nM). It has been recently proven in monkeys, using an osteoporosis model for in vivo evaluation of the 170-HSD2 inhibitor 3, that inhibition of this enzyme helps to maintain a sufficient local level of E2 and T in bones when the level of circulating active sex steroid drops (Bagi, C. M. et al., J. Musculoskelet. Neuronal Interact., 8:267-280 (2008)).

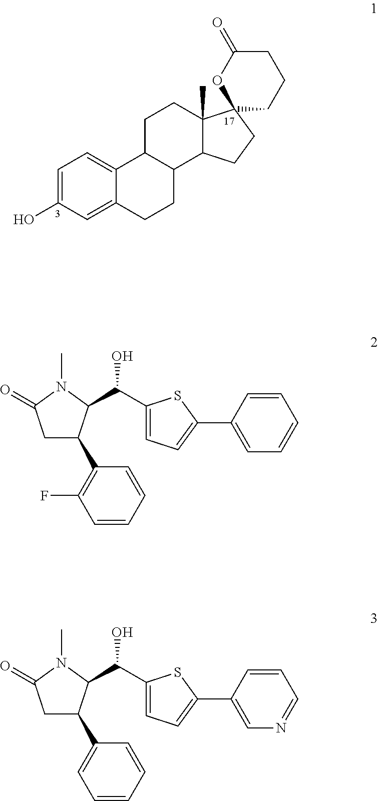

This modulation of steroid levels might be useful for a variety of indications like prevention and treatment of diseases caused by a misbalance of OB/OC activity like osteoporosis and osteopenia (Lindsay, R. et al., Obst. Gynecol., 76:290-295 (1990); Turner, R. T. et al., Endocr. Rev., 16:275-300 (1994); Meczekalski, B. et al., Gynecol. Endocrinol., 26:652-657 (2010)), post-menopausal symptoms, vaginal atrophy (Krychman, J. L. et al., J. Sex. Med., doi:10.1111/j.1743-6109.2010. 021148 (2010); Mac Bride, M. B., Mayo Clin. Proc., 85:87-94 (2010); Smith, A. L et al., Discov. Med., 10:500-510 (2010)); colon cancer (Oduwole O. O. et al., J. Steroid. Biochem. Mol. Biol. 87:133-140 (2003)), neuronal diseases (Behl, C. et al., Biochem. Biophys. Res. Commun., 216:473-482 (1995)) like Alzheimer (Pike, C. J., Front Neuroendocrinol., 30:239-258 (2009)) and Parkinson's disease (Bourque, M. et al., Front Neuroendocrinol., 30:142-157 (2009)), Depression (Schmidt, P. J. et al., Ann. N.Y. Acad. Sci., 179:70-85 (2009)) anxiety, hypercholesterolemia (Karjalainen, A. et al., Arterioscler. Thromb. Vasc. Biol., 4:1101-1106 (2000)), cardiovascular diseases (Traish, A. M. et al., J. Androl., 30:477-494 (2009); Xing D. et al., Arterioscler. Thromb. Vasc. Biol. 29:289-295 (2009)); hair loss (Mooradian, A. D. et al., Endocr. Rev., 8:1-28 (1987); Georgala S et al., Dermatology 208:178-9 (2004)), rheumatic diseases and inflammatory diseases (Cutolo, M. et al., Ann. N.Y. Sci., 1193:36-42 (2010); Islander U. et al., Mol. Cell. Endocrinol. Doi:10.1016/j.mce. 2010.05.018 (2010)).

### SHORT DESCRIPTION OF THE INVENTION

It was now found an improved series of new non-steroidal inhibitors which show, besides a high potency at the protein level (17β-HSD2: IC₅₀<100 nM, selectivity factor over 17β-HSD1 of up to >100 and very weak binding affinity to the estrogen receptors α and β: % RBA<0.1). The invention thus provides:

(1) A compound having 17beta-hydroxysteroid dehydrogenase type 2 (17β-HSD2) inhibitory activity of the formula (I)

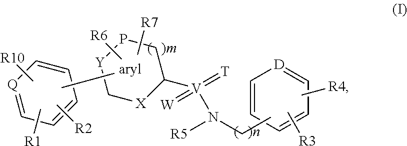

wherein

R1 and R3 are independently selected from H, —OH, alkoxy, acyloxy, alkyl, —N(R8)₂, —SR8, halogen, haloalkyl and phenyl;

R2, R4, R6 and R7 are independently selected from H, —ORS, —COR8, —COOR8, —CONHR8, —OCOR8, —SR8, —SON(R8)₂, —SO₂N(R8)₂, —N(R8)₂, —NHCOR8, —NHSOR8, —NHSO₂R8, -halogen, —NO₂, —CN, —SO₂R8, —CH₂N(R8)₂, —CH₂OR8, aryl optionally substituted with up to three R8, and alkyl optionally substituted with up to three R8;

R5 is lower alkyl;

R8 is selected from H, alkyl optionally substituted with up to three R9, —OH, —NO₂, lower alkoxy, lower alkyl, halogen, haloalkyl, —NH₂, —CN, —NHCOR9, —CONHR9, —NHSO₂R9, —SO₂NHR9, a 5- or 6-membered aromatic or heteroaromatic aryl ring optionally substituted with up to three R9, a homoaromatic ring that might be condensed with a 5- or 6-membered, aliphatic or aromatic heterocyclic ring and that (condensed) homoaromatic ring might optionally be substituted with up to three R9;

R9 is selected from —OH, alkoxy, halogen, haloalkyl, lower alkyl, —NH₂, —NO₂, —CN and phenyl;

R10 is selected from H, halogen, haloalkyl, lower alkyl;

m is an integer selected from 0 and 1;

n is an integer selected from 0 to 2;

the aryl ring is a 5- or 6-membered heteroaromatic ring which carries up to 3 heteroatoms X Y and P independently selected from N, S, O and said substituents R6 or R7 may be bound to heteroatoms or to ring carbon atoms;

X, Y and P are independently selected from CH, N, N(H), S and O;

Q and D are independently selected from CH and N;

V is C or S;

W is O or S; and

T, if V is S, represents 0 or is absent, or, if V is C, is absent; or a salt thereof.

(2) In a preferred embodiment of the aspect (1) above, the compound has the formula (II)

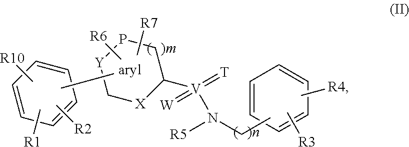

wherein the variables R1, R2, R3, R4, R5, R6, R7, R10, the aryl ring, n, m, X, Y, P, V, W and T have the same meaning as in (1) above, or a salt thereof.
(3) In a further preferred embodiment of the aspect (1) above, the compound has the formula (III)

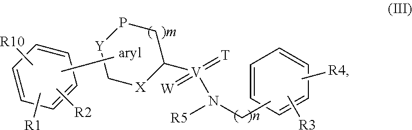

wherein the variables R1, R2, R3, R4, R5, R10, the aryl ring, n, m, X, Y, P, V, W and T have the same meaning as in (1) above, or a salt thereof.
(4) In a further preferred embodiment of the aspect (1) above, the compound has the formula (IV)

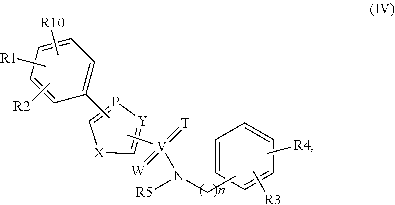

wherein the variables R1, R2, R3, R4, R5, R10, n, X, Y, P, V, W and T have the same meaning as in (1) above, or a salt thereof.
(5) In a preferred embodiment of the aspect (4) above, the compound has the formula (IVa)

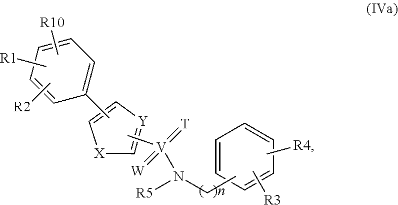

wherein the variables R1, R2, R3, R4, R5, R10, n, X, Y, V, W and T have the same meaning as in (1) above, or a salt thereof.
(6) In a preferred embodiment of the aspect (5) above, the compound has the formula (IVb)

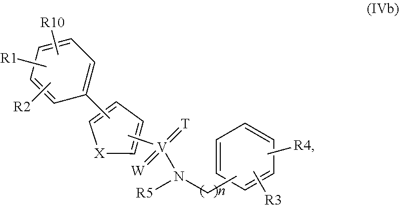

wherein the variables R1, R2, R3, R4, R5, R10, n, X, V, W and T have the same meaning as in (1) above, or a salt thereof.
(7) In a further preferred embodiment of the aspect (1) above, the compound has the formula (Va) or (Vb)

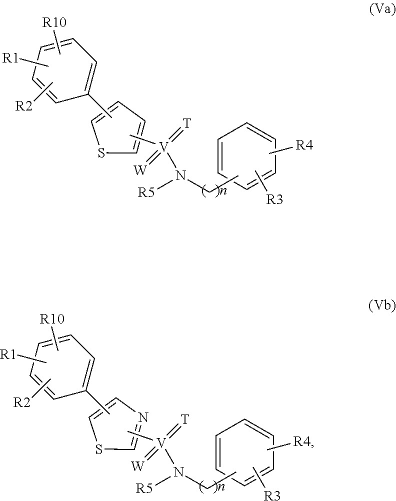

wherein the variables R1, R2, R3, R4, R5, R10, n, V, W and T have the same meaning as in (1) above, or a salt thereof.
(8) In a further preferred embodiment of the aspect (1) above, the compound has the formula (VIa) or (VIb)

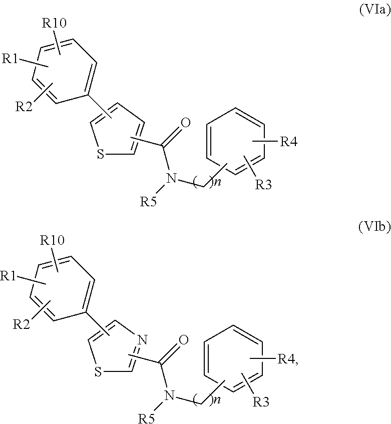

wherein the variables R1, R2, R3, R4, R5, R10, and n have the same meaning as in (1) above, or a salt thereof.
(9) In a further preferred embodiment of the aspect (1) above, the compound has the formula (VIIa) or (VIIb)

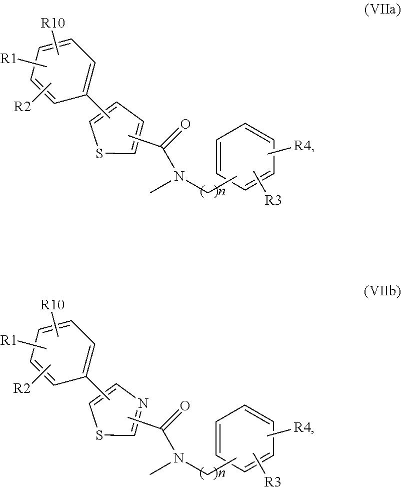

wherein the variables R1 and R3 are independently selected from H, —OH, —OMe, -Me, —NMe₂ and phenyl; R2, R4 and R10 are independently selected from H and halogen; and n is 0 or 1; or a salt thereof.
(10) In still a further preferred embodiment of the aspect (1) above, the compound has the formula (VIIIa), (VIIIb) or (VIIIc)

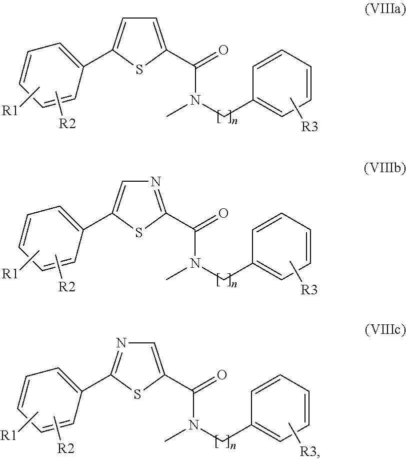

wherein R1 and R3 are independently selected from —OH, —OCH₃ and —CH₃; R2 is H, fluoro or methyl; and n is 0 or 1; or a salt thereof.
(11) A process for producing a compound formula (I) or a salt thereof according to any one of aspects (1) to (10) above, which comprises condensing an activated acyl or sulfonyl compound of the formula (B) with a secondary amine of the formula (C), followed by a cross-coupling reaction with a compound of the formula (A)

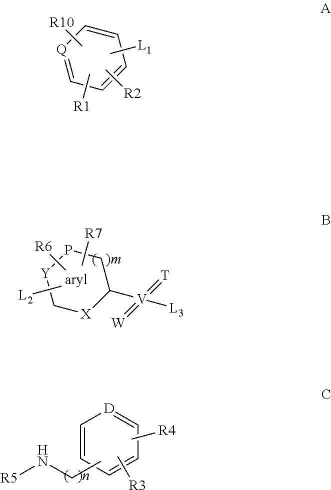

wherein all the variables are as defined in (1) above, and L1, L2 and L3 are leaving groups.
(12) A compound having 17β-HSD2 inhibitory activity according to any one of aspects (1) to (10) above or a pharmaceutically acceptable salt thereof for use in the treatment and/or prophylaxis of sex steroid deficient-diseases.
(13) A pharmaceutical composition or medicament comprising at least one compound of aspects (1) to (10) above or a pharmaceutically acceptable salt thereof, and optionally a suitable carrier or excipient.
(14) The use of a compound of aspects (1) to (10) above or a pharmaceutically acceptable salt thereof for preparing a medicament for the treatment and prophylaxis of osteoporotic diseases.
(15) A method for the treatment and prophylaxis of osteoporotic diseases in a patient which comprises administering the patient a suitable amount of a compound of aspects (1) to (10) above or a pharmaceutically acceptable salt thereof.

### DETAILED DESCRIPTION OF THE INVENTION

In the compounds of formula (I) of the invention, the variables and the terms used for their characterization have the following meanings:

“Alkyl residues” and “alkoxy residues” within the meaning of the invention may be straight-chain, branched-chain or cyclic, and saturated or (partially) unsaturated. Preferable alkyl residues and alkoxy residues are saturated or have one or more double and/or triple bonds. Of straight-chain or branched-chain alkyl residues, preferred are those having from 1 to 6 carbon atoms, preferably those having from 1 to 4 carbon atoms. Of the cyclic alkyl residues, more preferred are mono- or bicyclic alkyl residues having from 3 to 6 carbon atoms.

“Lower alkyl” and “lower alkoxy” residues within the meaning of the invention are straight-chain, branched-chain or cyclic saturated lower alkyl residues and lower alkoxy residues or those having a double or triple bond. Of the straight-chain ones, those having from 1 to 6 carbons atoms, especially 1 to 3 carbon atoms, are particularly preferred.

“Aryls” and “homocyclic aromatic groups” within the meaning of the present invention include, if not specified otherwise, mono-, bi- and tricyclic aryl residues having from 3 to 18 ring atoms, which may optionally be anellated with one or more saturated rings. Particularly preferred are anthracenyl, dihydronaphthyl, fluorenyl, hydrindanyl, indanyl, indenyl, naphthyl, naphthenyl, phenanthrenyl, phenyl and tetralinyl.

“Halogen” includes fluorine, chlorine, bromine and iodine.

The aryl in the formulas (I) to (III) include five- and six-membered arylene and heteroarylene residnes that are apparent to a skilled person, including phenyl, pyidin, pyrrol, furan, thiophen, imidazol, oxazol, thiazol, triazol etc.

“Salts” and “pharmaceutically acceptable salts” within the meaning of the present invention include salts of the compounds with organic acids (such as lactic acid, acetic acid, amino acid, oxalic acid, etc.), inorganic acids (such as HCl, HBr, phosphoric acid etc.), and, if the compounds have acid substituents, also with organic or inorganic bases including amino acids. Preferred are salts with HCl.

“Pharmacologically suitable carriers” within the meaning of the present invention are selected by the skilled person, depending on the desired dosage form.

In preferred embodiments of aspects (1) to (8) of the invention, the variables, if present, have the following meaning:

R1 and R3 are independently selected from H, —OH, alkoxy, alkyl, —N(R8)₂, halogen, haloalkyl and phenyl;

R2, R4, R6 and R7 are independently selected from H, —OR8, —COR8, —COOR8, —CONHR8, —OCOR8, —SO₂N(R8)₂, —N(R8)₂, —NHCOR8, —NHSO₂R8, halogen, —CH₂N(R8)₂, —CH₂OR8, aryl optionally substituted with up to three R8, C₁₋₆ alkyl optionally substituted with up to three R8 and halo C₁₋₆ alkyl;

R5 is lower alkyl, preferably is methyl, isopropyl or cyclopropyl;

R8 is selected from H, C₁₋₆ alkyl optionally substituted with up to three R9, halogen, halo C₁₋₆ alkyl, —OH, lower alkoxy, —NH₂, and 5- or 6-membered aromatic or heteroaromatic ring optionally substituted with up to three R9, preferably is phenyl or benzyl;

R9 is selected from —OH, alkoxy, halogen, haloalkyl, C₁₋₄ alkyl, —NH₂ and phenyl; X,

Y and P are independently selected from CH, N, N(H) and S; and/or

n is 0 or 1.

In the process of aspect (11) of the invention the leaving groups L1, L2 and L3 vary depending on the type of condensation reaction, but are selected to avoid any interference with each other (i.e. are “orthogonal” to allow the subsequent condensation reactions without cross reactions), or are ligands that can be converted into such orthogonal leaving groups. Preferably L1 is a —B(OH)₂ group, L2 is Br or OTf (trifluoromethanesulfonate) and L3 is Cl.

In a preferred embodiment of the inhibitor for use in the treatment and/or prophylaxis of sex steroid deficient-diseases of aspects (12) above, or in the pharmaceutical composition or medicament of aspects (13) and (14) above, or in the method of aspect (15) above is for the treatment and prophylaxis of sex steroid deficient diseases including diseases caused by a misbalance of OB/OC activity like osteoporosis and osteopenia, bone repair (such as bone fracture repair, bone healing, fracture healing, bone grafting, healing of bone implants and the like) post-menopausal symptoms, vaginal atrophy, colon cancer, neuronal diseases like Alzheimer and Parkinson's disease, depression, anxiety, hypercholesterolemia, cardiovascular diseases, hair loss, rheumatic diseases and inflammatory diseases, preferably for the treatment and prophylaxis of osteoporosis.

Particular preferred compound of aspects (1) to (10) above and preferred compounds for use in the treatment and/or prophylaxis of sex steroid deficient-diseases of aspects (10) above are the following compounds (1) to (85):

3′-Methoxy-N-(3-methoxybenzyl)-N-methylbiphenyl-4-carboxamide (1), 3′-hydroxy-N-(3-hydroxybenzyl)-N-methylbiphenyl-4-carboxamide (2), N-(3-methoxybenzyl)-N,3′-dimethylbiphenyl-4-carboxamide (3), N-(3-hydroxybenzyl)-N,3′-dimethylbiphenyl-4-carboxamide (4), 4′-Methoxy-N-(3-methoxybenzyl)-N-methylbiphenyl-3-carboxamide (5), 4′-hydroxy-N-(3-hydroxybenzyl)-N-methylbiphenyl-3-carboxamide (6), 3′-methoxy-N-(3-methoxybenzyl)-N-methylbiphenyl-3-carboxamide (7), 3′-hydroxy-N-(3-hydroxybenzyl)-N-methylbiphenyl-3-carboxamide (8), 4′-methoxy-N-(3-methoxyphenyl)-N-methylbiphenyl-3-carboxamide (9), 4′-hydroxy-N-(3-hydroxyphenyl)-N-methylbiphenyl-3-carboxamide (10), 3′-methoxy-N-(3-methoxyphenyl)-N-methylbiphenyl-3-carboxamide (11), 3′-hydroxy-N-(3-hydroxyphenyl)-N-methylbiphenyl-3-carboxamide (12), 2′-methoxy-N-(3-methoxyphenyl)-N-methylbiphenyl-3-carboxamide (13), 2′-hydroxy-N-(3-hydroxyphenyl)-N-methylbiphenyl-3-carboxamide (14), N-(3-methoxyphenyl)-N-methylbiphenyl-3-carboxamide (15), N-(3-hydroxyphenyl)-N-methylbiphenyl-3-carboxamide (16), N-(3-methoxyphenethyl)-5-(3-methoxyphenyl)-N-methylthiophene-2-carboxamide (17), N-(3-hydroxyphenethyl)-5-(3-hydroxyphenyl)-N-methylthiophene-2-carboxamide (18), N-(3-methoxyphenethyl)-N-methyl-5-phenylthiophene-2-carboxamide (19), N-(3-hydroxyphenethyl)-N-methyl-5-phenylthiophene-2-carboxamide (20), 5-(3-fluorophenyl)-N-(3-methoxyphenethyl)-N-methylthiophene-2-carboxamide (21), 5-(3-fluorophenyl)-N-(3-hydroxyphenethyl)-N-methylthiophene-2-carboxamide (22), N-(3-methoxybenzyl)-5-(3-methoxyphenyl)-N-methylthiophene-2-carboxamide (23), N-(3-hydroxybenzyl)-5-(3-hydroxyphenyl)-N-methylthiophene-2-carboxamide (24), N-(3-methoxybenzyl)-5-(4-methoxyphenyl)-N-methylthiophene-2-carboxamide (25), N-(3-hydroxybenzyl)-5-(4-hydroxyphenyl)-N-methylthiophene-2-carboxamide (26), N-(3-Methoxybenzyl)-5-(2-methoxyphenyl)-N-methylthiophene-2-carboxamide (27), N-(3-hydroxybenzyl)-5-(2-hydroxyphenyl)-N-methylthiophene-2-carboxamide (28), N-(3-methoxybenzyl)-N-methyl-5-phenylthiophene-2-carboxamide (29), N-(3-hydroxybenzyl)-N-methyl-5-phenylthiophene-2-carboxamide (30), 5-(4-cyanophenyl)-N-(3-methoxybenzyl)-N-methylthiophene-2-carboxamide (31), 5-(4-cyanophenyl)-N-(3-hydroxybenzyl)-N-methylthiophene-2-carboxamide (32), N-benzyl-5-(3-methoxyphenyl)-N-methylthiophene-2-carboxamide (33), N-benzyl-5-(3-hydroxyphenyl)-N-methylthiophene-2-carboxamide (34), N-benzyl-5-(4-methoxyphenyl)-N-methylthiophene-2-carboxamide (35), N-benzyl-5-(4-hydroxyphenyl)-N-methylthiophene-2-carboxamide (36), N-benzyl-5-(2-methoxyphenyl)-N-methylthiophene-2-carboxamide (37), N-benzyl-5-(2-hydroxyphenyl)-N-methylthiophene-2-carboxamide (38), N-(3-hydroxybenzyl)-5-(4-methoxyphenyl)-N-methylthiophene-2-carboxamide (39), 5-(3-(dimethylamino)phenyl)-N-(3-hydroxybenzyl)-N-methylthiophene-2-carboxamide (40), 5-(3-fluorophenyl)-N-(3-hydroxybenzyl)-N-methylthiophene-2-carboxamide (41), 5-(4-fluorophenyl)-N-(3-hydroxybenzyl)-N-methylthiophene-2-carboxamide (42), 5-(2-fluoro-3-methoxyphenyl)-N-(3-hydroxybenzyl)-N-methylthiophene-2-carboxamide (43), 5-(2-fluoro-3-hydroxyphenyl)-N-(3-hydroxybenzyl)-N-methylthiophene-2-carboxamide (44), N-(3-hydroxybenzyl)-5-(3-methoxyphenyl)-N-methylthiophene-2-carboxamide (45), N-(3-Hydroxybenzyl)-N-methyl-5-m-tolylthiophene-2-carboxamide (46), N,5-bis(3-methoxyphenyl)-N-methylthiophene-2-carboxamide (47), N,5-bis(3-hydroxyphenyl)-N-methylthiophene-2-carboxamide (48), 5-(2-methoxyphenyl)-N-(3-methoxyphenyl)-N-methylthiophene-2-carboxamide (49), 5-(2-hydroxyphenyl)-N-(3-hydroxyphenyl)-N-methylthiophene-2-carboxamide (50), N-(3-methoxyphenyl)-N-methyl-5-phenylthiophene-2-carboxamide (51), N-(3-hydroxyphenyl)-N-methyl-5-phenylthiophene-2-carboxamide (52), 5-(4-cyanophenyl)-N-(3-methoxyphenyl)-N-methylthiophene-2-carboxamide (53), 5-(4-cyanophenyl)-N-(3-hydroxyphenyl)-N-methylthiophene-2-carboxamide (54), N-(3-methoxyphenyl)-5-(4-methoxyphenyl)-N-methylthiophene-2-carboxamide (55), 5-(2-fluoro-3-methoxyphenyl)-N-(3-methoxyphenyl)-N-methylthiophene-2-carboxamide (56), 5-(2,4-dimethoxyphenyl)-N-(3-methoxyphenyl)-N-methylthiophene-2-carboxamide (57), 5-(3-(dimethylamino)phenyl)-N-(3-methoxyphenyl)-N-methylthiophene-2-carboxamide (58), 5-(3-fluorophenyl)-N-(3-methoxyphenyl)-N-methylthiophene-2-carboxamide (59), 5-(3,4-difluorophenyl)-N-(3-methoxyphenyl)-N-methylthiophene-2-carboxamide (60), 5-(3-fluoro-4-methoxyphenyl)-N-(3-methoxyphenyl)-N-methylthiophene-2-carboxamide (61), 5-(4-fluoro-3-methoxyphenyl)-N-(3-methoxyphenyl)-N-methylthiophene-2-carboxamide (62), N-(3-methoxyphenyl)-N-methyl-5-(3-(methylthio)phenyl)thiophene-2-carboxamide (63), N-(3-methoxyphenyl)-N-methyl-5-m-tolylthiophene-2-carboxamide (64), 5-(2,6-difluoro-3-methoxyphenyl)-N-(3-methoxyphenyl)-N-methylthiophene-2-carboxamide (65), 5-(2-fluoro-3-methylphenyl)-N-(3-methoxyphenyl)-N-methylthiophene-2-carboxamide (66), 5-(3-methoxyphenyl)-N-methyl-N-phenylthiophene-2-carboxamide (67), 5-(3-hydroxyphenyl)-N-methyl-N-phenylthiophene-2-carboxamide (68), N-methyl-N,5-diphenylthiophene-2-carboxamide (69), 5-(3-methoxyphenyl)-N-methyl-N-m-tolylthiophene-2-carboxamide (70), 5-(3-hydroxyphenyl)-N-methyl-N-m-tolylthiophene-2-carboxamide (71), 5-(2-fluoro-3-methoxyphenyl)-N-methyl-N-(m-tolylthiophene)-2-carboxamide (72), 5-(2-fluoro-3-methoxyphenyl)-N-(4-methoxyphenyl)-N-methylthiophene-2-carboxamide (73), N-(2-fluorophenyl)-N-methyl-5-m-tolylthiophene-2-carboxamide (74), 5-(3-methoxyphenyl)-N-methyl-N-(3-(trifluoromethyl)phenyl)thiophene-2-carboxamide (75), N-(biphenyl-3-yl)-5-(3-methoxyphenyl)-N-methylthiophene-2-carboxamide (76), 5-(2-fluoro-3-methoxyphenyl)-N-(3-methoxyphenyl)thiophene-2-carboxamide (77), 5-(2-fluoro-3-methoxyphenyl)-N-(3-methoxyphenyl)-N-phenylthiophene-2-carboxamide (78), N-(3-methoxyphenyl)-N-methyl-2-(3-methylphenyl)-1,3-thiazole-5-carboxamide (79), 2-(2-fluoro-3-methoxyphenyl)-N-(3-hydroxybenzyl)-N-methyl-1,3-thiazole-5-carboxamide (80), N-(3-methoxyphenyl)-N-methyl-5-(3-methylphenyl)-1,3-thiazole-2-carboxamide (81), N-cyclopropyl-N-(3-methoxyphenyl)-5-(3-methylphenyl)thiophene-2-carboxamide (82), N-cyclopropyl-5-(2-fluoro-3-methoxyphenyl)-N-(3-hydroxybenzyl)thiophene-2-carboxamide (83), N-(3-methoxyphenyl)-N-methyl-5-(3-methylphenyl)thiophene-2-sulfonamide (84) and 5-(2-fluoro-3-methoxyphenyl)-N-(3-hydroxybenzyl)-N-methylthiophene-2-sulfonamide (85), or a salt thereof.

Particularly preferred among compounds (1) to (85) are compounds (28), (30), (39), (43), (44), (46), (47), (55), (56), (58), (64), (65), (72), (76) and (81), or a salt thereof.

In the following the chemical structures of the particularly preferred inhibitors (1) to (78) of the invention (together with compounds (79)-(85) herein after also referred to as “title compounds”) are shown.

Title compounds: 1-22:

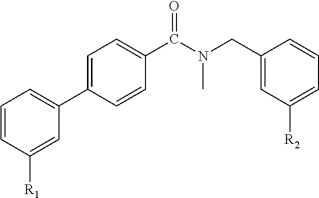

| cmpd | R₁ | R₂ |
|---|---|---|
| 1 | OCH₃ | OCH₃ |
| 2 | OH | OH |
| 3 | m-CH₃ | OCH₃ |
| 4 | m-CH₃ | OH |

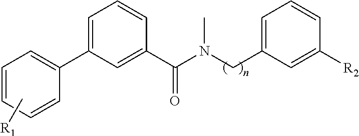

| cmpd | n | R₁ | R₂ |
|---|---|---|---|
| 5 | 1 | p-OCH₃ | OCH₃ |
| 6 | 1 | p-OH | OH |
| 7 | 1 | m-OCH₃ | OCH₃ |
| 8 | 1 | m-OH | OH |
| 9 | 0 | p-OCH₃ | OCH₃ |
| 10 | 0 | p-OH | OH |
| 11 | 0 | m-OCH₃ | OCH₃ |
| 12 | 0 | m-OH | OH |
| 13 | 0 | o-OCH₃ | OCH₃ |
| 14 | 0 | o-OH | OH |
| 15 | 0 | — | OCH₃ |
| 16 | 0 | — | OH |

| 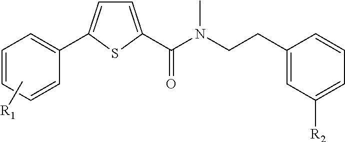 | | |
|---|---|---|
| cmpd | R₁ | R₂ |
| 17 | m-OCH₃ | OCH₃ |
| 18 | m-OH | OH |
| 19 | | OCH₃ |
| 20 | | OH |
| 21 | m-F | OCH₃ |
| 22 | m-F | OH |

Title compounds: 23-46:

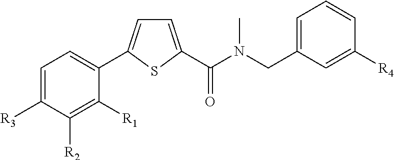

| cmpd | R₁ | R₂ | R₃ | R₄ |
|---|---|---|---|---|
| 23 | | OCH₃ | | OCH₃ |
| 24 | | OH | | OH |
| 25 | | | OCH₃ | OCH₃ |
| 26 | | | OH | OH |
| 27 | OCH₃ | | | OCH₃ |
| 28 | OH | | | OH |
| 29 | | | | OCH₃ |
| 30 | | | | OH |
| 31 | | | CN | OCH₃ |
| 32 | | | CN | OH |
| 33 | | OCH₃ | | |
| 34 | | OH | | |
| 35 | | | OCH₃ | |
| 36 | | | OH | |
| 37 | OCH₃ | | | |
| 38 | OH | | | |
| 39 | | | OCH₃ | OH |
| 40 | | N(Me)₂ | | OH |
| 41 | | F | | OH |
| 42 | | | F | OH |
| 43 | F | OCH₃ | | OH |
| 44 | F | OH | | OH |
| 45 | | OCH₃ | | OH |
| 46 | | CH₃ | | OH |

Title compounds: 47-78:

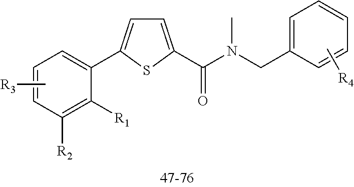

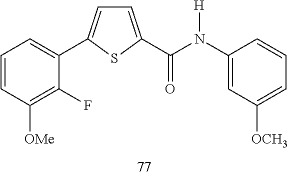 [SMILES: COc1cccc(NC(=O)c2ccc(-c3cccc(OC)c3F)s2)c1]

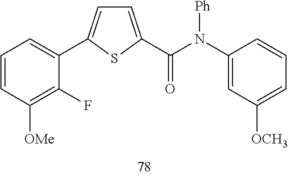 [SMILES: COc1cccc(N(C(=O)c2ccc(-c3cccc(OC)c3F)s2)c2ccccc2)c1]

| cmpd | R₁ | R₂ | R₃ | R₄ |
|---|---|---|---|---|
| 47 | | OCH₃ | | m-OCH₃ |
| 48 | | OH | | m-OH |
| 49 | OCH₃ | | | m-OCH₃ |
| 50 | OH | | | m-OH |
| 51 | | | | m-OCH₃ |
| 52 | | | | m-OH |
| 53 | | | p-CN | m-OCH₃ |
| 54 | | | p-CN | m-OH |
| 55 | | | p-OCH₃ | m-OCH₃ |
| 56 | F | OCH₃ | | m-OCH₃ |
| 57 | OCH₃ | | m-OCH₃ | m-OCH₃ |
| 58 | | N(CH₃)₂ | | m-OCH₃ |
| 59 | | F | | m-OCH₃ |
| 60 | | F | p-F | m-OCH₃ |
| 61 | | F | p-OCH₃ | m-OCH₃ |
| 62 | | OCH₃ | p-F | m-OCH₃ |
| 63 | | SCH₃ | | m-OCH₃ |
| 64 | | CH₃ | | m-OCH₃ |
| 65 | F | OCH₃ | o-F | m-OCH₃ |
| 66 | F | CH₃ | | m-OCH₃ |
| 67 | | OCH₃ | | |
| 68 | | OH | | |
| 69 | | | | |
| 70 | | OCH₃ | | m-CH₃ |
| 71 | | OH | | m-CH₃ |
| 72 | F | OCH₃ | | m-CH₃ |
| 73 | F | OCH₃ | | p-OCH₃ |
| 74 | | CH₃ | | o-F |
| 75 | | OCH₃ | | m-CF₃ |
| 76 | | OCH₃ | | m-ph |

The synthesis of the compounds of aspects (1) to (10) above according to aspect (11) of the invention is performed according to the following schemes:

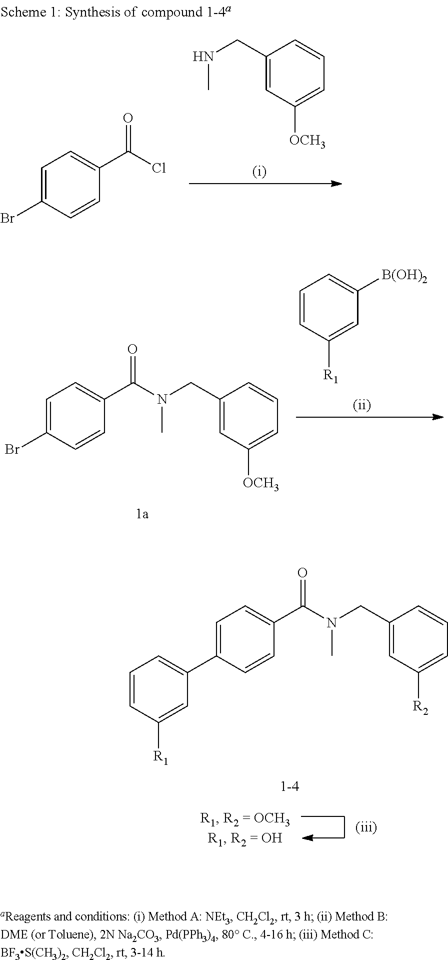

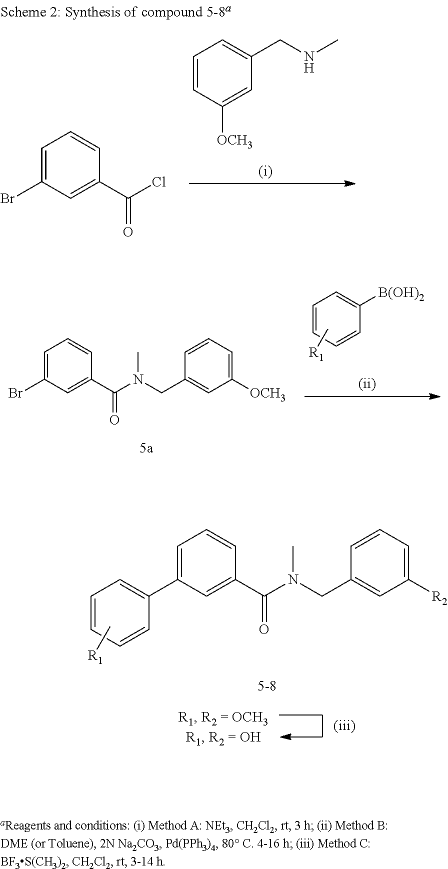

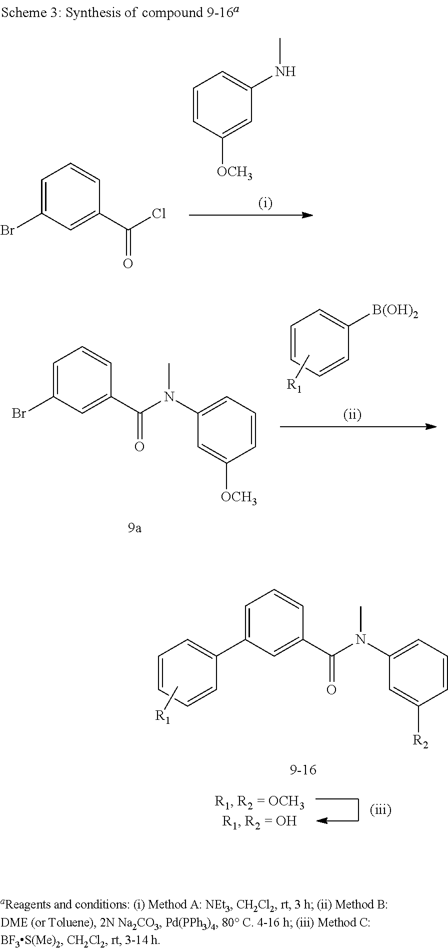

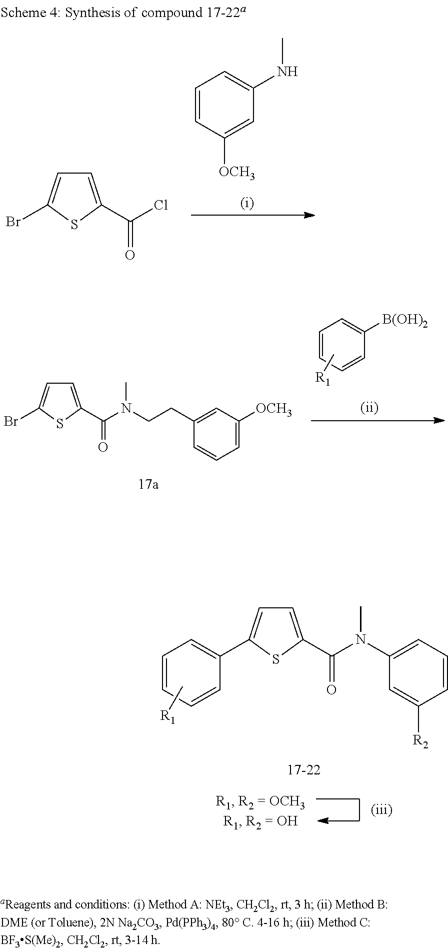

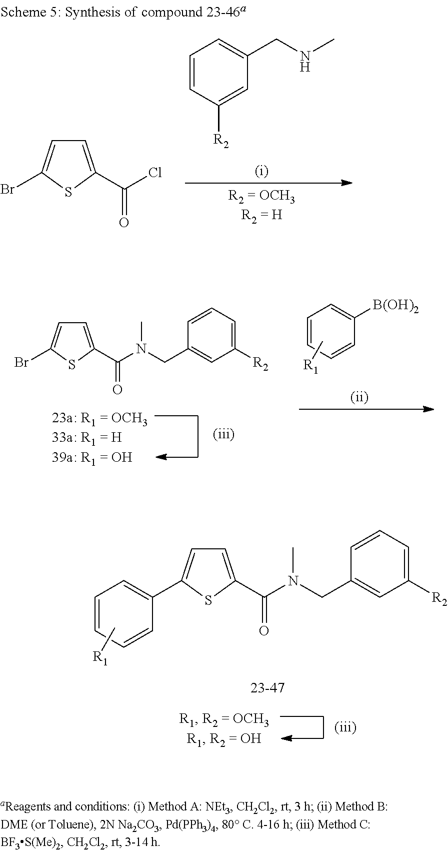

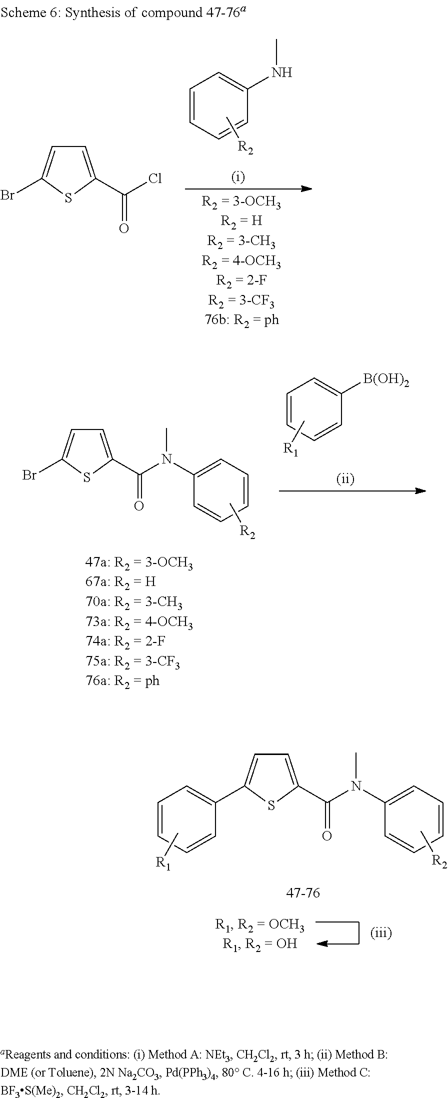

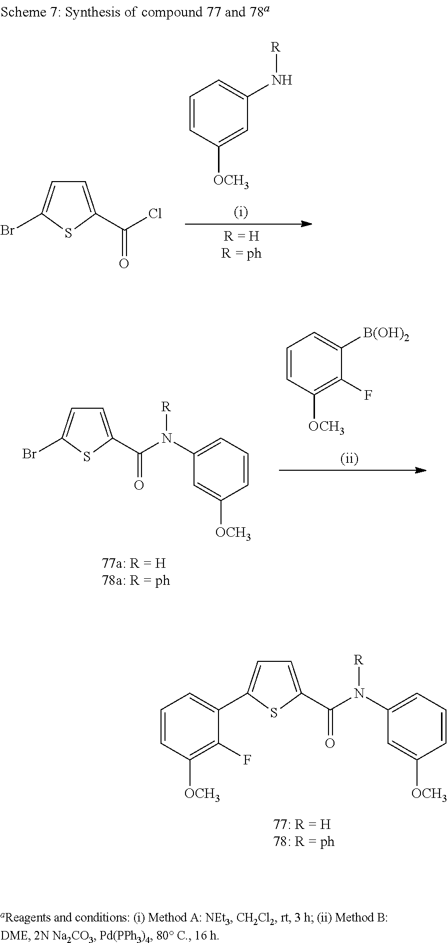

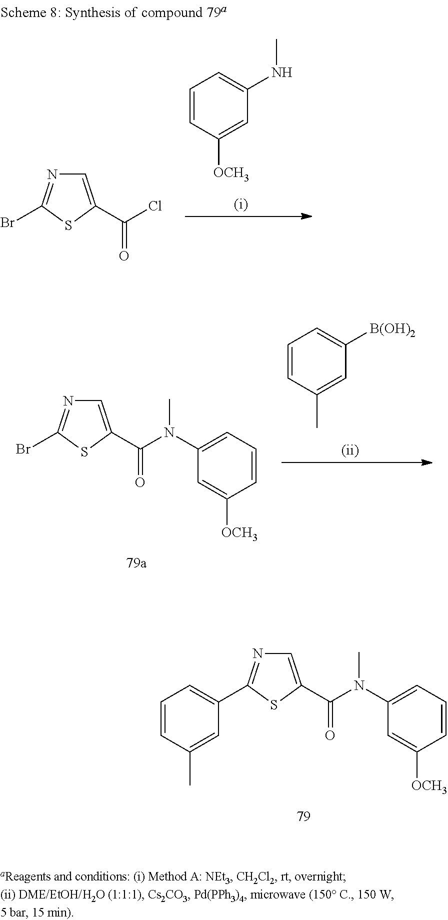

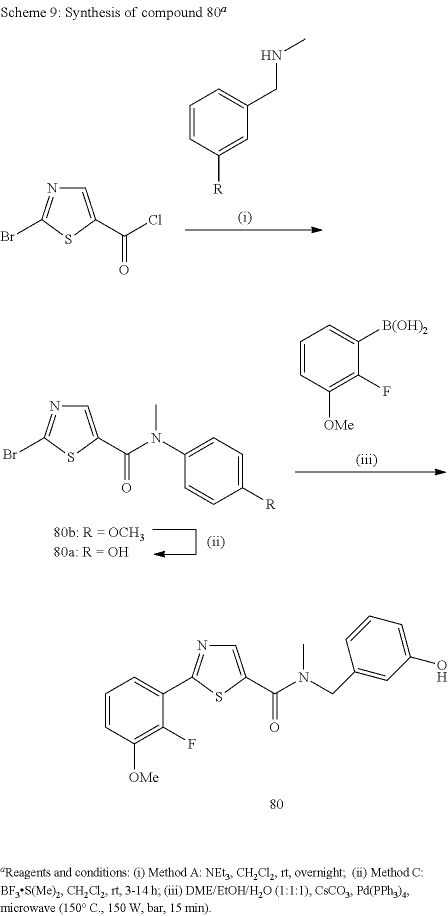

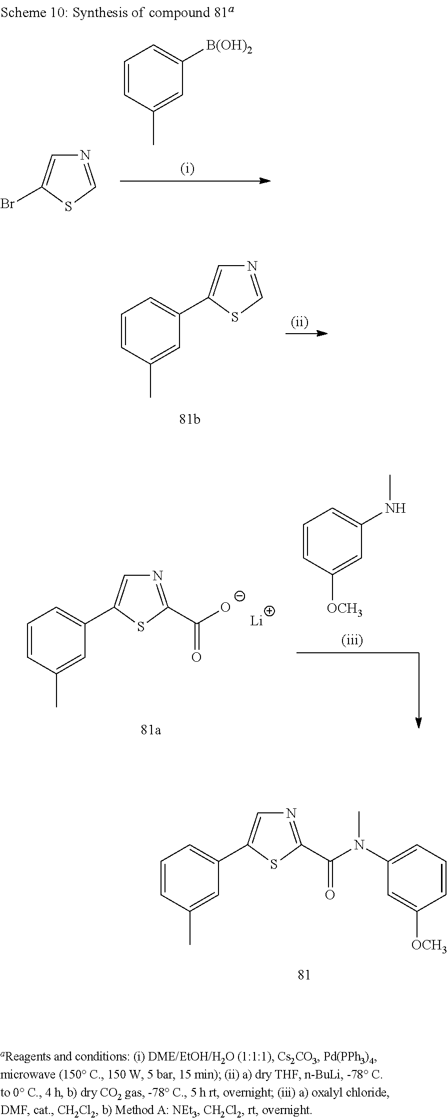

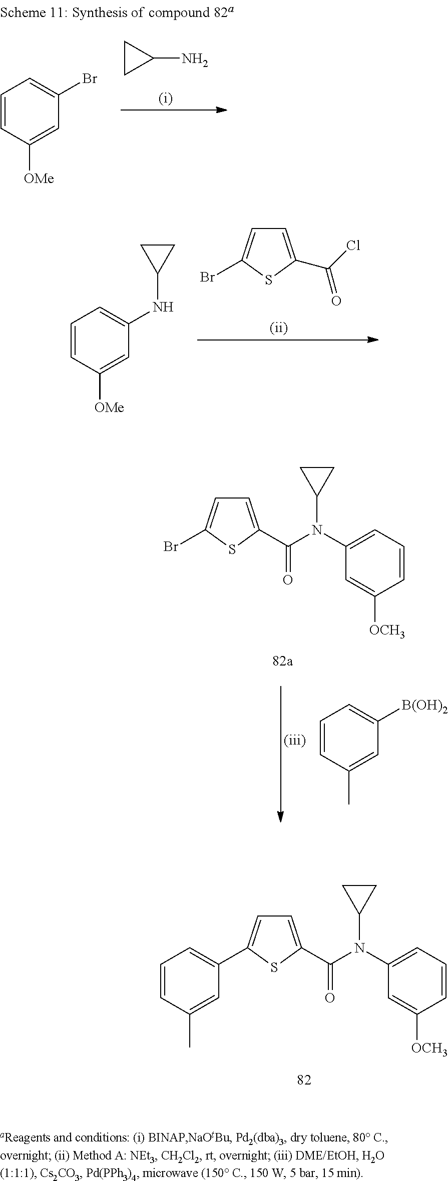

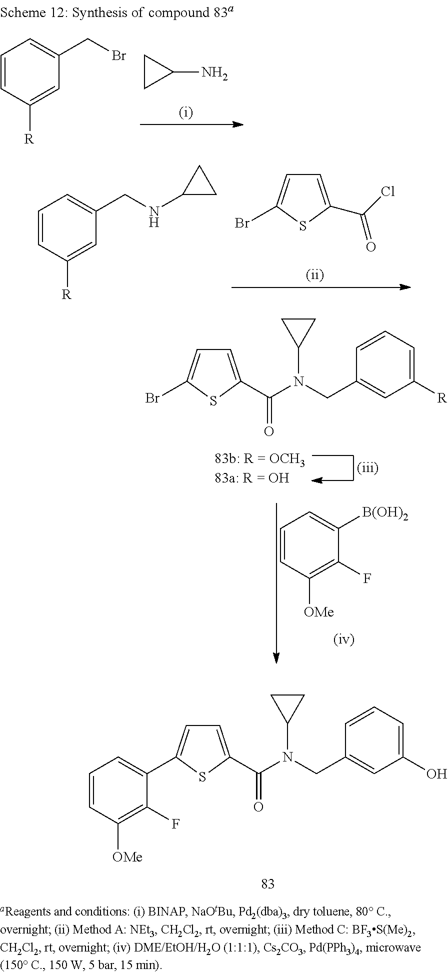

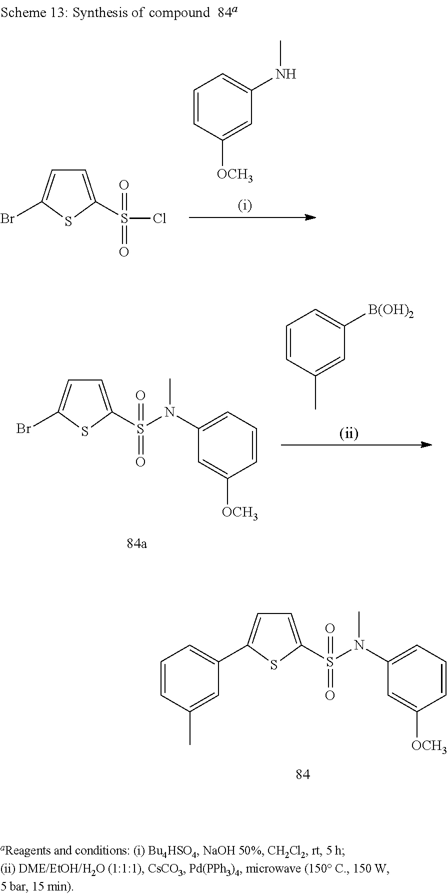

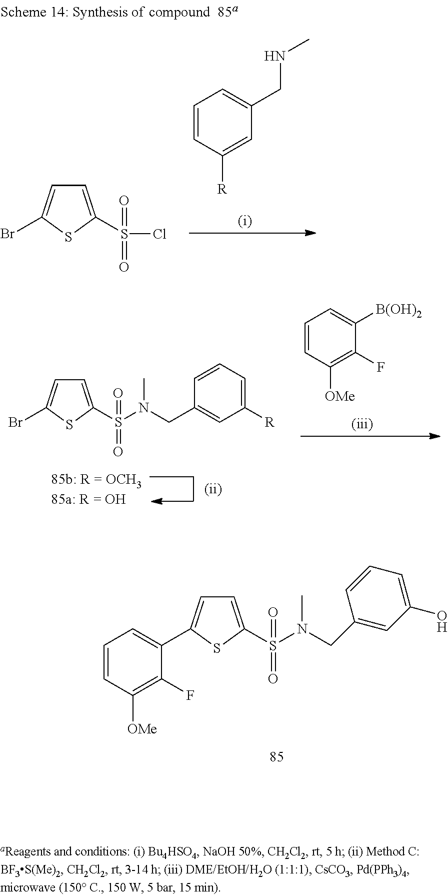

The invention is described in more detail in the following examples, which are however not to be construed as limiting the invention.

### EXAMPLES

### Materials and Methods

Chemical names follow IUPAC nomenclature. Starting materials were purchased from Aldrich, Acros, Lancaster, Maybridge, Combi Blocks, Merck or Fluka and were used without purification.

Flash chromatography (CC) was performed on silica gel (70-200 μm) coated with silica, preparative thin layer chromatography (TLC) on 1 mm SIL G-100 UV₂₅₄ glass plates (Macherey-Nagel) and reaction progress was monitored by TLC on Alugram SIL G UV₂₅₄ (Macherey-Nagel).

¹H-NMR spectra were measured on a Bruker AM500 spectrometer (500 MHz) at 300 K. Chemical shifts are reported in δ (parts per million: ppm), by reference to the hydrogenated residues of deuteriated solvent as internal standard (CDCl₃: δ=7.24 ppm (¹H-NMR) and δ=77 ppm (¹³C-NMR), CD₃OD: δ=5.84 ppm (¹H-NMR) and δ=49.3 ppm (¹³C-NMR), CD₃COCD₃: δ=2.05 ppm (¹H-NMR) and δ=30.8 ppm (¹³C-NMR), CD₃SOCD₃ δ=2.50 ppm (¹H-NMR) and δ=39.5 ppm (¹³C-NMR)). Signals are described as s, d, t, dd, ddd, m, dt, td and q for singlet, doublet, triplet, doublet of doublets, doublet of doublets of doublets, multiplet, doublet of triplets, triplet of doublets and quadruplets respectively. All coupling constants (J) are given in hertz (Hz).

Mass spectra (ESI) were recorded on a TSQ Quantum (Thermo Finnigan) instrument.

Tested compounds have >95% chemical purity as measured by HPLC. Several final compounds were purified via an Agilent Technologies Series 1200-preparative HPLC using a linear gradient run (solvents: acetonitrile, water) starting from 20% acetonitrile up to 100% over 36 min.

### Example 1

### Preparation of Title Compounds 1-78

Method A, General Procedure for Amidation:

At 0° C., a solution of bromoaryl carbonyl chloride (1 eq) in CH₂Cl₂ (2 ml/mol) was added drop wise to a solution of amine or N-substituted amine (1 eq) and triethylamine (1.15 eq) in CH₂Cl₂ (2 ml/mol). The mixture was kept stirring at 0° C. for 3 h and evaporated under reduced pressure. The residue was purified using flash chromatography (FC, n-hexane/ethyl acetate as eluent).

Method B, General Procedure for Suzuki Coupling:

Arylbromide (1 eq), (substituted)aryl boronic acid (1 eq), sodium carbonate (2 eq) and tetrakis(triphenylphosphine) palladium (0.1 eq) in an oxygen free DME/water (1:1) solution was stirred at 80° C. for 4 to 16 hours under nitrogen. The reaction mixture was cooled to rt. The aqueous layer was extracted with dichloromethane. The combined organic layers were washed with brine, dried over sodium sulfate, filtered and concentrated to dryness. The product was purified by FC with hexane/ethyl acetate (20:1-3:1) as eluent.

Method C, General Procedure for Ether Cleavage:

To a solution of methoxyphenyl compounds (1 eq) in dry dichloromethane (5 ml/mmol reactant), borontrifluoride dimethyl sulfide complex (6 eq per methoxy function) was added drop wise at 0° C. and stirred for 3 to 14 h. After the reaction was finished, the reaction mixture was diluted with dichloromethane and NaHCO₃ (5% aqueous) was added to neutralize. The aqueous layer was extracted with dichloromethane. The combined organic layers were washed with brine, dried over sodium sulfate, evaporated to dryness under reduced pressure. The product was purified by FC, with dichloromethane/methanol (100:1-50:1) or hexane/ethyl acetate (10:1-3:1) as eluent.

### 1.1. 3′-Methoxy-N-(3-methoxybenzyl)-N-methylbiphenyl-4-carboxamide (1)

### 4-Bromo-N-(3-methoxybenzyl)-N-methylbenzamide (1a)

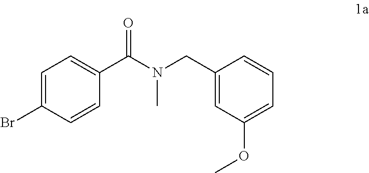 [SMILES: COc1cccc(CN(C)C(=O)c2ccc(Br)cc2)c1]

The title compound was prepared by reaction of 4-bromobenzoylchloride (329 mg, 1.5 mmol) and 3-methoxybenzylamine (227 mg, 1.5 mmol) in presence of triethylamine (0.24 ml, 1.73 mmol) according to method A. Purification by FC (n-hexane/ethyl acetate 6:1) afforded the desired product (468 mg, 93%); MS (ESI): 335 (M+H)⁺; ¹H NMR (CD₃COCD₃): 2.80-2.92 (m, 3H), 3.80 (s, 3H), 4.52-4.69 (m, 2H), 6.79 (br s, 1H), 6.86 (dd, J=8.5, 2.2 Hz, 1H), 6.95 (br s, 1H), 7.28 (t, J=7.9 Hz, 1H), 7.44 (d, J=7.9 Hz, 2H), 7.62 (br s, 2H); ¹³C NMR (CD₃COCD₃): 55.3, 112.9, 113.6, 120.0, 123.1, 129.1, 129.4, 130.0, 131.7, 135.7, 139.1, 159.8.

### 3′-Methoxy-N-(3-methoxybenzyl)-N-methylbiphenyl-4-carboxamide (1)

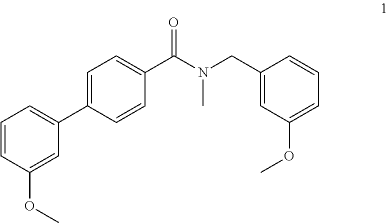 [SMILES: COc1cccc(CN(C)C(=O)c2ccc(-c3cccc(OC)c3)cc2)c1]

The title compound was prepared by reaction of 4-bromo-N-(3-methoxybenzyl)-N-methylbenzamide (1a) (67 mg, 0.2 mmol) and 3-methoxyphenylboronic acid (36 mg, 0.24 mmol) with tetrakis(triphenylphosphine) palladium (23 mg, 0.02 mmol) according to method B for 4 h. Purification by FC (n-hexane/ethyl acetate 6:1→3:1) afforded the desired compound (65 mg, 90%); MS (ESI): 362 (M+H)⁺; ¹H NMR (CD₃COCD₃): 2.97 (s, 3H), 3.81 (s, 3H), 3.87 (s, 3H), 4.61-4.72 (m, 2H), 6.85 (dd, J=8.2, 2.0 Hz, 2H), 6.96 (dd, J=8.2, 2.0 Hz, 2H), 7.23-7.26 (m, 2H), 7.30 (t, J=7.9 Hz, 1H), 7.38 (t, J=8.0 Hz, 1H), 7.57 (d, J=8.5 Hz, 2H), 7.72-7.73 (m, 2H); ¹³C NMR (CD₃COCD₃): 55.5, 55.6, 113.3, 113.6, 114.2, 120.1, 127.7, 128.5, 130.6, 130.8, 136.8, 142.5, 142.8, 161.1, 161.2.

### 1.2. 3′-Hydroxy-N-(3-hydroxybenzyl)-N-methylbiphenyl-4-carboxamide (2)

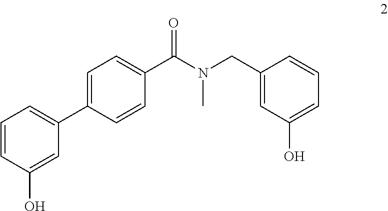 [SMILES: CN(Cc1cccc(O)c1)C(=O)c1ccc(-c2cccc(O)c2)cc1]

The title compound was prepared by reaction of 3′-methoxy-N-(3-methoxybenzyl)-N-methylbiphenyl-4-carboxamide (1) (36 mg, 0.1 mmol) with borontrifluoride dimethyl sulfide complex (0.13 ml, 1.2 mmol) according to method C for 14 h. Purification by FC(CH₂Cl₂/CH₃OH 100:1→50:1) yielded the title compound (28 mg, 84%); MS: 334 (M+H)⁺; ¹H NMR (CD₃COCD₃): 2.95 (s, 3H), 4.57-4.60 (m, 2H), 6.75 (dd, J=8.2, 2.0 Hz, 2H), 6.85 (dd, J=8.2, 2.0 Hz, 2H), 7.14-7.15 (m, 2H), 7.20 (t, J=8.0 Hz, 1H), 7.29 (t, J=8.0 Hz, 1H), 7.55 (d, J=8.2 Hz, 2H), 7.67 (br s, 2H), 8.38 (br s, 1H), 8.48 (s, 1H); ¹³C NMR (CD₃COCD₃): 114.7, 115.2, 115.7, 119.0, 127.6, 128.4, 130.4, 130.9, 136.7, 142.5, 142.9, 158.8, 158.9.

### 1.3. N-(3-Methoxybenzyl)-N,3′-dimethylbiphenyl-4-carboxamide (3)

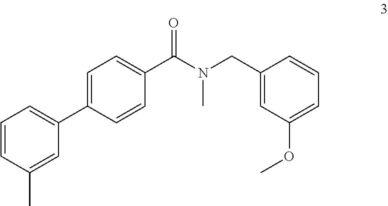 [SMILES: COc1cccc(CN(C)C(=O)c2ccc(-c3cccc(C)c3)cc2)c1]

The title compound was prepared by reaction of 4-bromo-N-(3-methoxybenzyl)-N-methylbenzamide (1a) (53 mg, 0.15 mmol) and 3-methylphenylboronic acid (25 mg, 0.23 mmol) with tetrakis(triphenylphosphine) palladium (17 mg, 0.015 mmol) according to method B for 6 h. Purification by FC (n-hexane/ethyl acetate 6:1→3:1) afforded the desired compound (50 mg, 95%); MS (ESI): 346 (M+H)⁺; ¹H NMR (CD₃COCD₃): 2.40 (s, 3H), 2.97 (s, 3H), 3.80 (s, 3H), 4.60-4.72 (m, 2H), 6.81-6.98 (m, 3H), 7.20 (d, J=7.3 Hz, 1H), 7.30 (t, J=8.0 Hz, 1H), 7.35 (t, J=7.6 Hz, 1H), 7.47 (d, J=7.6 Hz, 1H), 7.51 (s, 1H), 7.56 (d, J=8.5 Hz, 2H), 7.71 (d, J=7.3 Hz, 2H); ¹³C NMR (CD₃COCD₃): 21.5, 55.5, 113.6, 124.9, 127.6, 128.5, 129.3, 129.7, 130.6, 136.6, 139.3, 141.0, 143.0, 161.1.

### 1.4. N-(3-hydroxybenzyl)-N,3′-dimethylbiphenyl-4-carboxamide (4)

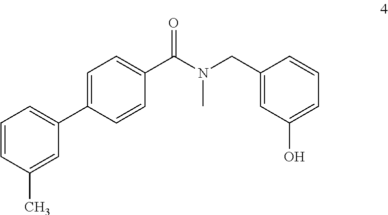 [SMILES: Cc1cccc(-c2ccc(C(=O)N(C)Cc3cccc(O)c3)cc2)c1]

The title compound was prepared by reaction of N-(3-methoxybenzyl)-N,3′-dimethylbiphenyl-4-carboxamide (3) (35 mg, 0.1 mmol) with borontrifluoride dimethyl sulfide complex (0.06 ml, 0.6 mmol) according to method C for 6 h. purification by FC(CH₂Cl₂/CH₃OH 100:1→50:1) yielded the title compound (30 mg, 89%); MS: 332 (M+H)⁺; ¹H NMR (CD₃COCD₃): 2.40 (s, 3H), 2.96 (s, 3H), 4.55-4.70 (m, 2H), 6.69-6.89 (m, 3H), 7.20 (t, J=7.9 Hz, 2H), 7.35 (t, J=7.6 Hz, 1H), 7.47 (d, J=7.9 Hz, 1H), 7.51 (s, 1H), 7.56 (d, J=8.2 Hz, 2H), 7.70-7.71 (m, 2H), 8.36 (br s, 1H); ¹³C NMR (CD₃COCD₃): 21.5, 115.2, 124.9, 127.6, 128.5, 129.3, 129.7, 136.7, 139.3, 141.0, 143.0.

### 1.5. 4′-Methoxy-N-(3-methoxybenzyl)-N-methylbiphenyl-3-carboxamide (5)

### 3-Bromo-N-(3-methoxybenzyl)-N-methylbenzamide (5a)

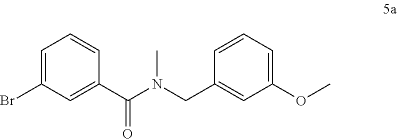 [SMILES: COc1cccc(CN(C)C(=O)c2cccc(Br)c2)c1]

The title compounds was prepared by reaction of 3-bromobenzoylchloride (217 mg, 1 mmol) and N-methyl-3-methoxybenzylamine (151 mg, 1 mmol) in presence of triethylamine (0.16 ml, 1.15 mmol) according to method A. The product was purified by FC (n-hexane/ethyl acetate 6:1), yielded 320 mg (98%) of the required compound; MS (ESI): 334 (M+H)⁺; ¹H NMR (CDCl₃): 2.86-3.03 (m, 3H), 3.81 (s, 3H), 4.45-4.71 (m, 2H), 6.69-6.74 (m, 1H), 6.85 (d, J=8.0 Hz, 1H), 6.89-6.92 (m, 1H), 7.24-7.26 (m, 1H), 7.28 (t, J=7.9 Hz, 1H), 7.37 (d, J=7.9 Hz, 1H), 7.53 (s, 1H), 7.60 (s, 1H); ¹³C NMR (CDCl₃): 50.8, 55.2, 112.5, 112.9, 118.9, 120.5, 122.6, 125.2, 125.5, 129.7, 130.0, 132.7, 138.2.

### 4′-Methoxy-N-(3-methoxybenzyl)-N-methylbiphenyl-3-carboxamide (5)

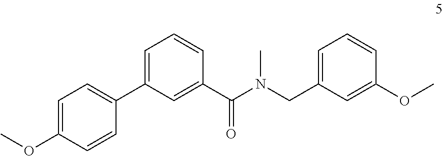

The title compound was prepared by reaction of 3-bromo-N-(3-methoxybenzyl)-N-methylbenzamide (5a) (67 mg, 2 mmol) and 4-methoxyphenylboronic acid (36 mg, 2.4 mmol) with tetrakis(triphenylphosphine) palladium (17 mg, 0.015 mmol) as catalyst according to method B for 4 h. Purification by FC (n-hexane/ethyl acetate 6:1) afforded the desired compound (70 mg, 97%); MS: 362 (M+H)⁺; ¹H NMR (CD₃COCD₃): 2.97 (br s, 3H), 3.79 (s, 3H), 3.84 (s, 3H), 4.57-4.73 (m, 2H), 6.83-6.88 (m, 5H), 7.30 (t, J=7.9 Hz, 1H), 7.40 (dt, J=7.9, 1.3 Hz, 1H), 7.48-7.68 (m, 5H); ¹³C NMR (CD₃COCD₃): 55.5, 55.6, 113.6, 114.5, 115.2, 125.7, 126.0, 128.1, 128.9, 129.7, 130.7, 133.4, 138.5, 141.6, 160.6.

### 1.6. 4′-Hydroxy-N-(3-hydroxybenzyl)-N-methylbiphenyl-3-carboxamide (6)

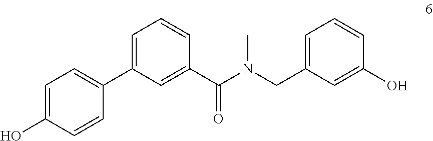 [SMILES: CN(Cc1cccc(O)c1)C(=O)c1cccc(-c2ccc(O)cc2)c1]

The title compound was prepared by reaction of 4′-methoxy-N-(3-methoxybenzyl)-N-methylbiphenyl-3-carboxamide (5) (40 mg, 0.11 mmol) with borontrifluoride dimethyl sulfide complex (0.14 ml, 1.32 mmol) according to method C for 4 h. Purification by FC(CH₂Cl₂/CH₃OH 100:1→50:1) yielded the title compound (30 mg, 82%); MS: 334 (M+H)⁺; ¹H NMR (CD₃COCD₃): 2.93-3.01 (m, 3H), 4.53-4.71 (m, 2H), 6.71-6.92 (m, 5H), 7.19 (t, J=7.9 Hz, 1H), 7.36-7.51 (m, 4H), 7.63 (br s, 1H), 7.66 (s, 1H), 8.51-8.52 (m, 1H), 8.62 (br s, 1H); ¹³C NMR (CD₃COCD₃): 115.2, 116.7, 125.5, 125.7, 128.1, 128.9, 129.7, 130.7, 132.2, 138.1, 139.9, 141.9, 158.3, 158.7.

### 1.7. 3′-Methoxy-N-(3-methoxybenzyl)-N-methylbiphenyl-3-carboxamide (7)

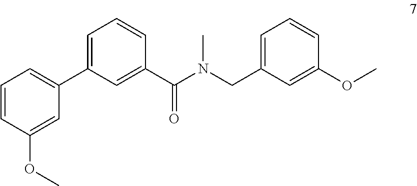 [SMILES: COc1cccc(CN(C)C(=O)c2cccc(-c3cccc(OC)c3)c2)c1]

The title compound was prepared by reaction of 3-bromo-N-(3-methoxybenzyl)-N-methylbenzamide (5a) (67 mg, 0.2 mmol) and 3-methoxyphenylboronic acid (36 mg, 0.24 mmol) with tetrakis(triphenylphosphine) palladium (23 mg, 0.02 mmol) as catalyst according to method B for 4 h. Purification by FC (n-hexane/ethyl acetate 6:1) afforded the desired compound (71 mg, 98%); ¹H NMR (CD₃COCD₃): 2.98 (br s, 3H), 3.79 (s, 3H), 3.85 (s, 3H), 4.58-4.73 (m, 2H), 6.81 (br s, 1H), 6.86 (dd, J=7.9, 2.2 Hz, 1H), 6.93-6.99 (m, 2H), 7.13 (br s, 1H), 7.22 (br s, 1H), 7.29 (t, J=7.9 Hz, 1H), 7.36 (br s, 1H), 7.46 (dt, J=7.6, 1.6 Hz, 1H), 7.52 (br s, 1H), 7.71 (br s, 1H), 7.73 (s, 1H); ¹³C NMR (CD₃COCD₃): 54.9, 55.5, 55.6, 88.6, 113.2, 113.6, 114.2, 120.1, 126.2, 126.8, 128.7, 129.8, 130.5, 130.8, 138.5, 140.1, 141.8, 142.5, 161.1, 161.2.

### 1.8. 3′-Hydroxy-N-(3-hydroxybenzyl)-N-methylbiphenyl-3-carboxamide (8)

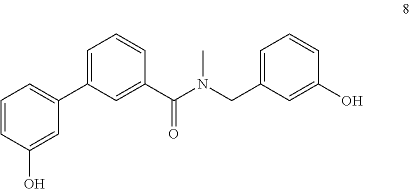 [SMILES: CN(Cc1cccc(O)c1)C(=O)c1cccc(-c2cccc(O)c2)c1]

The title compound was prepared by reaction of 3′-methoxy-N-(3-methoxybenzyl)-N-methylbiphenyl-3-carboxamide (7) (54 mg, 0.15 mmol) with borontrifluoride dimethyl sulfide complex (0.19 ml, 1.8 mmol) according to method C for 5 h, purification by FC(CH₂Cl₂/CH₃OH 100:1→50:1) yielded the title compound (46 mg, 92%); MS (ESI): 334 (M+H)⁺; ¹H NMR (CD₃COCD₃): 2.96 (br s, 3H), 4.54-4.70 (m, 2H), 6.74-6.91 (m, 4H), 7.02-7.27 (m, 4H), 7.44 (d, J=7.6 Hz, 1H), 7.51 (br s, 1H), 7.67 (br s, 1H), 7.69 (s, 1H), 8.37 (br s, 1H), 8.45 (s, 1H); ¹³C NMR (CD₃COCD₃): 114.7, 115.2, 115.6, 119.0, 119.9, 126.2, 126.7, 128.6, 129.7, 130.9, 138.4, 139.9, 142.0, 142.5, 158.7, 158.8.

### 1.9. 4′-Methoxy-N-(3-methoxyphenyl)-N-methylbiphenyl-3-carboxamide (9)

### 3-Bromo-N-(3-methoxyphenyl)-N-methylbenzamide (9a)

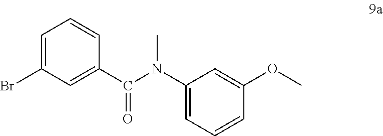 [SMILES: COc1cccc(N(C)C(=O)c2cccc(Br)c2)c1]

The title compound was prepared by reaction of 3-bromobenzoylchloride (217 mg, 1 mmol) and 3-methoxy-N-methylaniline (137 mg, 1 mmol) in presence of triethylamine (0.16 ml, 1.15 mmol) according to method A. Purification by FC (n-hexane/ethyl acetate 6:1) afforded the desired compound (300 mg, 94%); ¹H NMR (CD₃COCD₃): 3.41 (s, 3H), 3.71 (s, 3H), 6.74-6.77 (m, 2H), 6.80 (t, J=2.0 Hz, 1H), 7.15 (t, J=8.0 Hz, 1H), 7.18 (t, J=8.0 Hz, 1H), 7.27 (ddd, J=7.9, 2.0, 0.9 Hz, 1H), 7.45 (ddd, J=7.9, 2.0, 0.9 Hz, 1H), 7.52 (t, J=2.0 Hz, 1H); ¹³C NMR (CD₃COCD₃): 38.2, 55.7, 113.4, 113.9, 120.2, 122.0, 128.0, 130.5, 130.7, 132.2, 133.0, 140.0, 146.8, 161.2, 168.8.

### 4′-Methoxy-N-(3-methoxyphenyl)-N-methylbiphenyl-3-carboxamide (9)

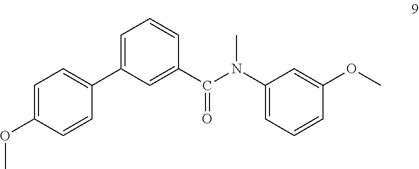 [SMILES: COc1ccc(-c2cccc(C(=O)N(C)c3cccc(OC)c3)c2)cc1]

The title compound was prepared by reaction of 3-bromo-N-(3-methoxyphenyl)-N-methylbenzamide (9a) (80 mg, 0.25 mmol) and 4-methoxyphenylboronic acid (45 mg, 0.3 mmol) with tetrakis(triphenylphosphine) palladium (29 mg, 0.025 mmol) as catalyst according to method B for 4 h. Purification by FC (n-hexane/ethyl acetate 10:1→6:1) afforded the desired compound (82 mg, 94%); MS (ESI): 348 (M+H)⁺; ¹H NMR (CD₃COCD₃): 3.44 (s, 3H), 3.69 (s, 3H), 3.82 (s, 3H), 6.77 (ddd, J=8.0, 2.5, 0.9 Hz, 1H), 6.79 (ddd, J=8.0, 2.2, 0.9 Hz, 1H), 6.82 (t, J=2.2 Hz, 1H), 6.97 (d, J=8.8 Hz, 2H), 7.19 (t, J=8.0 Hz, 1H), 7.26-7.31 (m, 2H), 7.38 (d, J=8.8 Hz, 2H), 7.50 (dt, J=7.3, 1.9 Hz, 1H), 7.52-7.53 (m, 1H); ¹³C NMR (CD₃COCD₃): 38.2, 55.6, 55.7, 113.1, 114.0, 115.1, 120.2, 127.6, 128.1, 128.7, 129.1, 130.6, 133.5, 138.2, 140.8, 147.5, 160.5, 161.2, 170.3.

### 1.10. 4′-Hydroxy-N-(3-hydroxyphenyl)-N-methylbiphenyl-3-carboxamide (10)

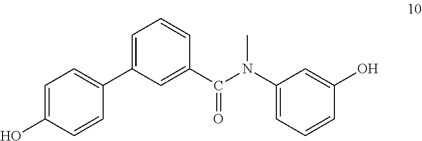 [SMILES: CN(C(=O)c1cccc(-c2ccc(O)cc2)c1)c1cccc(O)c1]

The title compound was prepared by reaction of 4′-methoxy-N-(3-methoxyphenyl)-N-methylbiphenyl-3-carboxamide (9) (35 mg, 0.1 mmol) with borontrifluoride dimethyl sulfide complex (0.13 ml, 1.2 mmol) according to method C for 8 h. Purification by FC(CH₂Cl₂/CH₃OH 100:1→50:1) yielded the title compound (28 mg, 88%); ¹H NMR (CD₃COCD₃): 3.42 (s, 3H), 6.67 (ddd, J=6.9, 2.0, 0.9 Hz, 1H), 6.68-6.69 (m, 2H), 6.88 (d, J=8.8 Hz, 2H), 7.09-7.12 (m, 1H), 7.24-7.29 (m, 2H), 7.31 (d, J=8.8 Hz, 2H), 7.47 (dt, J=6.9, 2.0 Hz, 1H), 7.52 (td, J=1.6, 0.6 Hz, 1H), 8.50 (s, 1H), 8.55 (s, 1H); ¹³C NMR (CD₃COCD₃): 39.3, 115.5, 116.2, 117.6, 120.2, 128.4, 128.5, 129.1, 129.8, 129.9, 131.7, 133.4, 138.9, 142.1, 148.4, 159.2, 160.0, 171.4.

### 1.11. 3′-Methoxy-N-(3-methoxyphenyl)-N-methylbiphenyl-3-carboxamide (11)

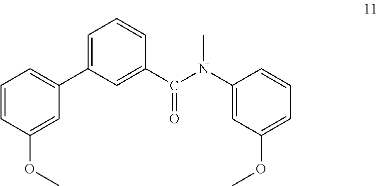 [SMILES: COc1cccc(-c2cccc(C(=O)N(C)c3cccc(OC)c3)c2)c1]

The title compound was prepared by reaction of 3-bromo-N-(3-methoxyphenyl)-N-methylbenzamide (9a) (80 mg, 0.25 mmol) and 3-methoxyphenylboronic acid (45 mg, 0.3 mmol) with tetrakis(triphenylphosphine) palladium (29 mg, 0.025 mmol) as catalyst according to method B for 4 h. Purification by FC (n-hexane/ethyl acetate 10:1→6:1) afforded the desired compound (85 mg, 98%); MS (ESI): 348 (M+H)⁺; ¹H NMR (CD₃COCD₃): 3.45 (s, 3H), 3.70 (s, 3H), 3.83 (s, 3H), 6.76 (dd, J=8.2, 2.5 Hz, 1H), 6.78 (dd, J=7.9, 1.3 Hz, 1H), 6.83 (t, J=2.5 Hz, 1H), 6.90 (ddd, J=8.2, 2.5, 0.6 Hz, 1H), 6.92 (t, J=2.1 Hz, 1H), 7.01 (d, J=7.8 Hz, 1H), 7.19 (t, J=8.2 Hz, 1H), 7.30-7.34 (m, 2H), 7.39 (dt, J=7.8, 1.3 Hz, 1H), 7.53-7.55 (m, 2H); ¹³C NMR (CD₃COCD₃): 39.2, 56.6, 56.7, 114.0, 114.1, 114.9, 115.2, 121.0, 121.2, 129.1, 129.5, 129.7, 130.2, 131.6, 131.7, 139.2, 142.0, 143.6, 148.4, 162.1, 162.2, 171.2.

### 1.12. 3′-Hydroxy-N-(3-hydroxyphenyl)-N-methylbiphenyl-3-carboxamide (12)

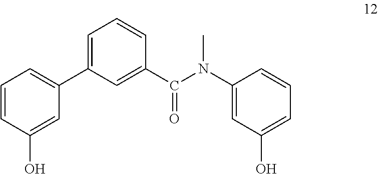 [SMILES: CN(C(=O)c1cccc(-c2cccc(O)c2)c1)c1cccc(O)c1]

The title compound was prepared by reaction of 3′-methoxy-N-(3-methoxyphenyl)-N-methylbiphenyl-3-carboxamide (11) (75 mg, 0.22 mmol) with borontrifluoride dimethyl sulfide complex (0.28 ml, 2.64 mmol) according to method C for 8 h.

Purification by FC(CH₂Cl₂/CH₃OH 100:1→50:1) yielded the title compound as colorless solid (65 mg, 94%); MS (ESI): 320 (M+H)⁺; ¹H NMR (CD₃COCD₃): 3.42 (s, 3H), 6.65-6.68 (m, 2H), 6.70 (ddd, J=7.9, 2.5, 0.9 Hz, 1H), 6.82 (ddd, J=7.9, 2.5, 0.9 Hz, 1H), 6.91 (ddd, J=7.6, 2.0, 0.9 Hz, 1H), 6.97 (t, J=2.0 Hz, 1H), 7.11 (td, J=7.9, 0.9 Hz, 1H), 7.23 (t, J=7.9 Hz, 1H), 7.29 (td, J=7.6, 0.9 Hz, 1H), 7.32 (dt, J=7.6, 1.6 Hz, 1H), 7.51 (ddd, J=7.3, 1.9, 1.2 Hz, 1H), 7.57 (td, J=1.9, 0.9 Hz, 1H), 8.36 (s, 1H), 8.45 (s, 1H); ¹³C NMR (CD₃COCD₃): 39.3, 115.5, 115.6, 116.2, 116.4, 120.0, 120.2, 129.0, 129.3, 129.6, 130.0, 131.7, 139.2, 142.2, 143.6, 148.4, 159.7, 159.9, 171.2.

### 1.13. 2′-Methoxy-N-(3-methoxyphenyl)-N-methylbiphenyl-3-carboxamide (13)

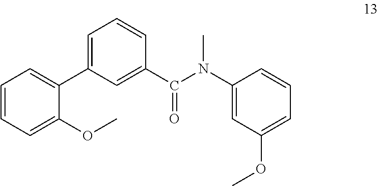 [SMILES: COc1cccc(N(C)C(=O)c2cccc(-c3ccccc3OC)c2)c1]

The title compound was prepared by reaction of 3-bromo-N-(3-methoxyphenyl)-N-methylbenzamide (9a) (80 mg, 0.25 mmol) and 2-methoxyphenylboronic acid (45 mg, 0.3 mmol) with tetrakis(triphenylphosphine) palladium (29 mg, 0.025 mmol) as catalyst according to method B for 12 h. Purification by FC (n-hexane/ethyl acetate 10:1→6:1) afforded the desired compound as colorless oil (79 mg, 91%); ¹H NMR (CD₃COCD₃): 3.44 (s, 3H), 3.69 (s, 3H), 3.75 (s, 3H), 6.74-6.78 (m, 3H), 6.96 (td, J=7.4, 1.0 Hz, 1H), 7.02 (dd, J=7.6, 1.9 Hz, 1H), 7.05 (dd, J=8.0, 0.9 Hz, 1H), 7.17-7.21 (m, 1H), 7.24 (td, J=8.0, 1.0 Hz, 1H), 7.28-7.32 (m, 2H), 7.43-7.45 (m, 2H); ¹³C NMR (CD₃COCD₃): 38.2, 55.7, 55.9, 112.5, 113.0, 113.9, 120.0, 121.6, 127.7, 128.0, 129.8, 130.4, 130.6, 131.3, 131.4, 137.5, 139.0, 147.5, 157.4, 161.2, 170.5.

### 1.14. 2′-Hydroxy-N-(3-hydroxyphenyl)-N-methylbiphenyl-3-carboxamide (14)

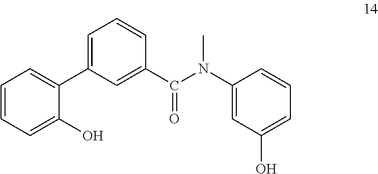 [SMILES: CN(C(=O)c1cccc(-c2ccccc2O)c1)c1cccc(O)c1]

The title compound was prepared by reaction of 2′-methoxy-N-(3-methoxyphenyl)-N-methylbiphenyl-3-carboxamide (13) (60 mg, 0.17 mmol) with borontrifluoride dimethyl sulfide complex (0.21 ml, 2.04 mmol) according to method C for 14 h. Purification by FC(CH₂Cl₂/CH₃OH 100:1→50:1) yielded the title compound as colorless solid (50 mg, 91%); MS (ESI): 320 (M+H)⁺; ¹H NMR (CD₃COCD₃): 3.41 (s, 3H), 6.65-6.68 (m, 3H), 6.86 (td, J=7.9, 1.3 Hz, 1H), 6.95 (dd, J=7.9, 1.3 Hz, 1H), 7.03, (dd, J=7.6, 1.6 Hz, 1H), 7.09 (td, J=7.9, 0.6 Hz, 1H), 7.15 (ddd, J=7.9, 7.0, 1.6 Hz, 1H), 7.24 (td, J=7.6, 0.6 Hz, 1H), 7.29 (dt, J=7.0, 1.6 Hz, 1H), 7.55-7.57 (m, 2H), 8.29, (s, 1H), 8.49 (s, 1H); ¹³C NMR (CD₃COCD₃): 38.4, 114.4, 115.1, 117.0, 119.2, 120.8, 127.7, 128.0, 128.5, 129.6, 130.3, 130.7, 131.3, 131.4, 137.4, 139.0, 147.4, 155.0, 158.9, 170.5.

### 1.15. N-(3-Methoxyphenyl)-N-methylbiphenyl-3-carboxamide (15)

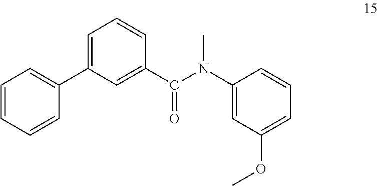 [SMILES: COc1cccc(N(C)C(=O)c2cccc(-c3ccccc3)c2)c1]

The title compound was prepared by reaction of 3-bromo-N-(3-methoxyphenyl)-N-methylbenzamide (9a) (80 mg, 0.25 mmol) and phenylboronic acid (36 mg, 0.3 mmol) with tetrakis(triphenylphosphine) palladium (29 mg, 0.025 mmol) as catalyst according to method B for 4 h. Purification by FC (n-hexane/ethyl acetate 10:1→6:1) afforded the desired compound as colorless oil (75 mg, 95%); MS (ESI): 318 (M+H)⁺; ¹H NMR (CD₃COCD₃): 3.45 (s, 3H), 3.70 (s, 3H), 6.76 (ddd, J=8.2, 2.5, 0.9 Hz, 1H), 6.80 (ddd, J=7.9, 2.2, 0.9 Hz, 1H), 6.83 (t, J=2.2 Hz, 1H), 7.20 (t, J=8.2 Hz, 1H), 7.30-7.37 (m, 3H), 7.39-7.45 (m, 4H), 7.55 (dt, J=7.6, 1.6 Hz, 1H), 7.57 (t, J=1.6 Hz, 1H).

### 1.16. N-(3-Hydroxyphenyl)-N-methylbiphenyl-3-carboxamide (16)

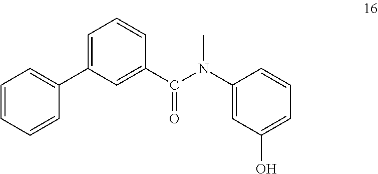 [SMILES: CN(C(=O)c1cccc(-c2ccccc2)c1)c1cccc(O)c1]

The title compound was prepared by reaction of N-(3-methoxyphenyl)-N-methyl biphenyl-3-carboxamide (15) (50 mg, 0.16 mmol) with borontrifluoride dimethyl sulfide complex (0.10 ml, 0.96 mmol) according to method C for 6 h. Purification by FC(CH₂Cl₂/CH₃OH 100:1→50:1) yielded the title compound as colorless solid (40 mg, 84%); MS (ESI): 304 (M+H)⁺; ¹H NMR (CD₃COCD₃): 3.43 (s, 3H), 6.66-6.70 (m, 3H), 7.11 (t, J=8.2 Hz, 1H), 7.30-7.38 (m, 3H), 7.39-7.42 (m, 2H), 7.44-7.46 (m, 2H), 7.55 (dt, J=7.6, 1.6 Hz, 1H), 7.58 (t, J=1.6 Hz, 1H), 8.52 (s, 1H).

### 1.17. N-(3-Methoxyphenethyl)-5-(3-methoxyphenyl)-N-methylthiophene-2-carboxamide (17)

### 2-(3-Methoxyphenyl)-N-methylethanamine (17b)

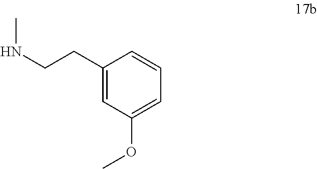

To a solution of THF (0.2 ml) and methylamine (1.9 ml, 1.205 g, 22 mmol, 40% in water) at room temperature was added a solution of 3-methoxyphenethyl bromide (215 mg, 1 mmol) in THF (0.8 ml) over 20 min. The reaction mixture was kept stirring at rt overnight and evaporated. The residue was purified using FC (eluent:CH₂Cl₂/CH₃OH (50:1-30:1)), and yielded the title compound as colorless solid (145 mg, 88%); MS (ESI): 166 (M+H)⁺; ¹H NMR (CD₃COCD₃): 2.06-2.09 (m, 4H), 3.28 (t, J=7.9 Hz, 2H), 3.38 (t, J=7.9 Hz, 2H), 3.78 (s, 3H), 6.81 (dd, J=2.4, 8.0 Hz, 1H), 6.90 (d, J=7.6 Hz, 1H), 6.95 (s, 1H), 7.22 (t, J=8.0 Hz, 1H); ¹³C NMR (CD₃COCD₃): 32.7, 33.5, 51.1, 55.5, 113.4, 115.3, 121.8, 130.6, 139.6, 161.0.

### 5-Bromo-N-(3-methoxyphenethyl)-N-methylthiophene-2-carboxamide (17a)

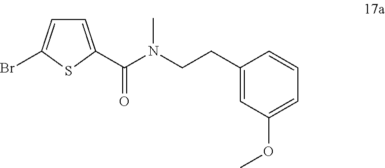 [SMILES: COc1cccc(CCN(C)C(=O)c2ccc(Br)s2)c1]

The title compound was prepared from 5-bromothiophene-2-carbonyl chloride (171 mg, 0.76 mmol) and 2-(3-methoxyphenyl)-N-methylethanamine (17b) (125 mg, 0.76 mmol) in presence of triethylamine (0.12 ml, 1.11 mmol) according to method A. Purification by FC(CH₂Cl₂) yielded the desired compound as colorless oil (235 mg, 94%). The product was directly in the next step without further analysis.

### N-(3-Methoxyphenethyl)-5-(3-methoxyphenyl)-N-methylthiophene-2-carboxamide (17)

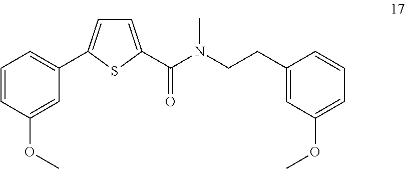 [SMILES: COc1cccc(CCN(C)C(=O)c2ccc(-c3cccc(OC)c3)s2)c1]

The title compound was prepared by reaction of 5-bromo-N-(3-methoxyphenethyl)-N-methylthiophene-2-carboxamide (17a) (78 mg, 0.22 mmol) and 3-methoxyphenylboronic acid (41 mg, 0.27 mmol) with tetrakis(triphenylphosphine) palladium (25 mg, 0.02 mmol) as catalyst according to method B for 6 h. Purification by FC (n-hexane/ethyl acetate 10:1->6:1) afforded the desired compound as colorless solid (65 mg, 78%); ¹H NMR (CD₃COCD₃): 2.96 (t, J=7.5 Hz, 2H), 3.19 (br s, 3H), 3.76 (s, 3H), 3.79 (t, J=7.5 Hz, 2H), 3.87 (s, 3H), 6.78 (ddd, J=8.2, 2.5, 0.9 Hz, 1H), 6.84 (br s, 1H), 6.85 (br s, 1H), 6.94 (ddd, J=8.2, 2.5, 0.9 Hz, 1H), 7.19-7.23 (m, 2H), 7.26 (ddd, J=7.8, 1.5, 0.9 Hz, 1H), 7.32-7.37 (m, 2H), 7.41 (d, J=3.8 Hz, 1H).

### 1.18. N-(3-Hydroxyphenethyl)-5-(3-hydroxyphenyl)-N-methylthiophene-2-carboxamide (18)

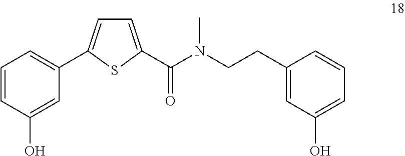 [SMILES: CN(CCc1cccc(O)c1)C(=O)c1ccc(-c2cccc(O)c2)s1]

The title compound was prepared by reaction of N-(3-methoxyphenethyl)-5-(3-methoxyphenyl)-N-methylthiophene-2-carboxamide (17)(45 mg, 0.12 mmol) with borontrifluoride dimethyl sulfide complex (0.16 ml, 1.44 mmol) according to method C for 14 h. Purification by FC(CH₂Cl₂/CH₃OH 100:1→50:1) yielded the title compound as colorless solid (35 mg, 84%); ¹H NMR (CD₃COCD₃): 2.91 (t, J=7.6 Hz, 2H), 3.19 (br s, 3H), 3.76 (t, J=7.6 Hz, 2H), 6.70 (ddd, J=8.0, 2.5, 0.9 Hz, 1H), 6.73-6.76 (m, 2H), 6.84 (ddd, J=8.0, 2.5, 0.9 Hz, 1H), 7.12 (t, J=7.9 Hz, 1H), 7.15-7.18 (m, 2H), 7.26 (t, J=7.9 Hz, 1H), 7.34 (s, 1H), 7.35 (s, 1H), 8.21 (s, 1H), 8.54 (s, 1H); ¹³C NMR (CD₃COCD₃): 113.4, 113.5, 114.2, 116.3, 118.0, 120.8, 123.8, 130.3, 131.1, 135.8, 158.5, 158.9.

### 1.19. N-(3-Methoxyphenethyl)-N-methyl-5-phenylthiophene-2-carboxamide (19)

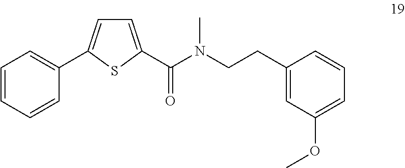 [SMILES: COc1cccc(CCN(C)C(=O)c2ccc(-c3ccccc3)s2)c1]

The title compound was prepared by reaction of 5-bromo-N-(3-methoxyphenethyl)-N-methylthiophene-2-carboxamide (17a) (75 mg, 0.21 mmol) and phenylboronic acid (31 mg, 0.25 mmol) with tetrakis(triphenylphosphine) palladium (24 mg, 0.02 mmol) as catalyst according to method B for 6 h. Purification by FC (n-hexane/ethyl acetate 10:1→6:1) afforded the desired compound as colorless solid (60 mg, 82%); ¹H NMR (CD₃COCD₃): 2.96 (t, J=7.6 Hz, 2H), 3.19 (br s, 3H), 3.76 (s, 3H), 3.79 (t, J=7.6 Hz, 2H), 6.78 (ddd, J=8.2, 2.5, 0.9 Hz, 1H), 6.84-6.85 (m, 2H), 7.21 (t, J=8.8 Hz, 1H), 7.34-7.38 (m, 2H), 7.40 (d, J=3.8 Hz, 1H), 7.42-7.46 (m, 2H), 7.69-7.71 (m, 2H); ¹³C NMR (CD₃COCD₃): 55.4, 112.8, 115.2, 121.9, 123.9, 126.7, 129.2, 130.0, 130.3, 134.5, 160.9.

### 1.20. N-(3-Hydroxyphenethyl)-N-methyl-5-phenylthiophene-2-carboxamide (20)

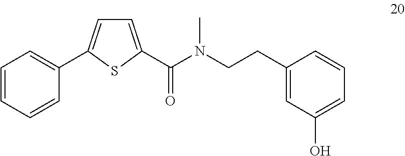 [SMILES: CN(CCc1cccc(O)c1)C(=O)c1ccc(-c2ccccc2)s1]

The title compound was prepared by reaction of N-(3-methoxyphenethyl)-N-methyl-5-phenylthiophene-2-carboxamide (19) (45 mg, 0.13 mmol) with borontrifluoride dimethyl sulfide complex (0.08 ml, 0.78 mmol) according to method C for 14 h. Purification by FC(CH₂Cl₂/CH₃OH 100:1→50:1) yielded the title compound as colorless solid (38 mg, 88%); ¹H NMR (CD₃COCD₃): 2.91 (t, J=7.6 Hz, 2H), 3.19 (br s, 3H), 3.76 (t, J=7.6 Hz, 2H), 6.70 (ddd, J=8.0, 2.5, 0.9 Hz, 1H), 6.73-6.76 (m, 2H), 7.12 (t, J=8.0 Hz, 1H), 7.34-7.38 (m, 2H), 7.40 (d, J=3.8 Hz, 1H), 7.42-7.46 (m, 2H), 7.69-7.72 (m, 2H), 8.21 (s, 1H); ¹³C NMR (CD₃COCD₃): 114.2, 116.6, 120.8, 123.9, 126.7, 129.1, 130.0, 130.3, 134.5, 134.5, 158.5.

### 1.21. 5-(3-Fluorophenyl)-N-(3-methoxyphenethyl)-N-methylthiophene-2-carboxamide (21)

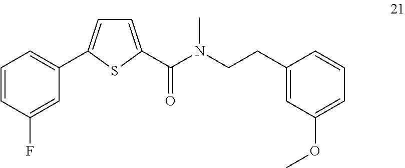 [SMILES: COc1cccc(CCN(C)C(=O)c2ccc(-c3cccc(F)c3)s2)c1]

The title compound was prepared by reaction of 5-bromo-N-(3-methoxyphenethyl)-N-methylthiophene-2-carboxamide (17a) (71 mg, 0.2 mmol) and 3-fluorophenylboronic acid (34 mg, 0.24 mmol) with tetrakis(triphenylphosphine) palladium (23 mg, 0.02 mmol) as catalyst according to method B for 6 h. Purification by FC (n-hexane/ethyl acetate 10:1→6:1) afforded the desired compound as colorless solid (60 mg, 81%); MS (ESI): 370 (M+H)⁺; ¹H NMR (CD₃COCD₃): 2.96 (t, J=7.5 Hz, 2H), 3.19 (br s, 3H), 3.76 (s, 3H), 3.79 (t, J=7.5 Hz, 2H), 6.78 (ddd, J=0.9, 2.5, 8.2 Hz, 1H), 6.83-6.85 (m, 2H), 7.13 (m, 1H), 7.21 (t, J=8.2 Hz, 1H), 7.36 (br s, 1H), 7.46-7.50 (m, 3H), 7.53 (dt, J=1.3, 7.8 Hz, 1H); ¹³C NMR (CD₃COCD₃): 55.4, 112.8, 113.1, 113.3, 115.3, 115.6, 115.8, 121.9, 122.7, 122.7, 124.9, 130.3, 131.9, 132.0, 136.9, 160.9, 165.1.

### 1.22. 5-(3-Fluorophenyl)-N-(3-hydroxyphenethyl)-N-methylthiophene-2-carboxamide (22)

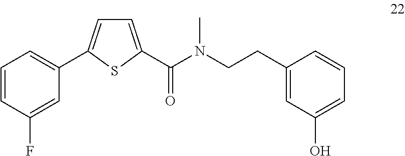 [SMILES: CN(CCc1cccc(O)c1)C(=O)c1ccc(-c2cccc(F)c2)s1]

The title compound was prepared by reaction of 5-(3-fluorophenyl)-N-(3-methoxyphenethyl)-N-methylthiophene-2-carboxamide (21) (40 mg, 0.11 mmol) with borontrifluoride dimethyl sulfide complex (0.07 ml, 0.66 mmol) according to method C for 14 h. Purification by FC(CH₂Cl₂/CH₃OH 100:1→50:1) yielded the title compound as colorless solid (30 mg, 78%); MS (ESI): 356 (M+H)⁺; ¹H NMR (CD₃COCD₃): 2.91 (t, J=7.5 Hz, 2H), 3.19 (br s, 3H), 3.76 (t, J=7.5 Hz, 2H), 6.70 (ddd, J=8.2, 2.5, 0.9 Hz, 1H), 6.73-6.76 (m, 2H), 7.10-7.15 (m, 2H), 7.36 (br s, 1H), 7.46-7.50 (m, 3H), 7.53 (dt, J=7.8, 1.2 Hz, 1H), 8.21 (s, 1H); ¹³C NMR (CD₃COCD₃): 113.1, 113.3, 114.2, 115.6, 115.8, 116.6, 120.71, 122.6, 122.7, 124.9, 130.3, 131.9, 132.0, 136.8, 158.6, 163.1, 165.0.

### 1.23. N-(3-Methoxybenzyl)-5-(3-methoxyphenyl)-N-methylthiophene-2-carboxamide (23)

### 5-Bromo-N-(3-methoxybenzyl)-N-methylthiophene-2-carboxamide (23a)

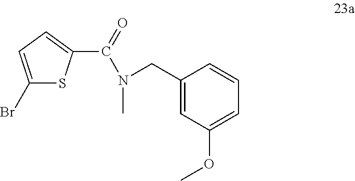 [SMILES: COc1cccc(CN(C)C(=O)c2ccc(Br)s2)c1]

The title compound was prepared by reaction of 5-bromothiophene-2-carbonyl chloride (225 mg, 1 mmol) and N-methyl-3-methoxybenzylamine (151 mg, 1 mmol) in presence of triethylamine (0.16 ml, 1.15 mmol) according to method A. The product was purified by FC (n-hexane/ethyl acetate 6:1), yielded (335 mg, 99%) as colorless oil; MS (ESI): 339 (M+H)⁺; ¹H NMR (CDCl₃): 3.11 (br s, 3H), 3.80 (s, 3H), 4.72 (br s, 2H), 6.80 (s, 1H), 6.83-6.86 (dd, J=8.0, 2.0 Hz, 2H), 6.96 (br s, 1H), 7.07 (br s, 1H), 7.29 (t, J=8.0 Hz, 1H); ¹³C NMR (CDCl₃): 55.2, 112.9, 117.2, 129.4, 129.8, 138.1, 139.5, 153.1, 160.1, 163.3.

### N-(3-Methoxybenzyl)-5-(3-methoxyphenyl)-N-methylthiophene-2-carboxamide (23)

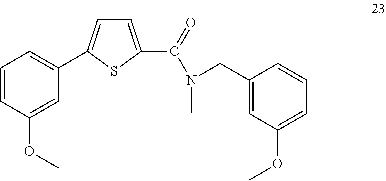 [SMILES: COc1cccc(CN(C)C(=O)c2ccc(-c3cccc(OC)c3)s2)c1]

The title compound was prepared by reaction of 5-bromo-N-(3-methoxybenzyl)-N-methylthiophene-2-carboxamide (23a) (124 mg, 0.37 mmol) and 3-methoxyphenylboronic acid (67 mg, 0.44 mmol) with tetrakis(triphenylphosphine) palladium (43 mg, 0.04 mmol) as catalyst according to method B for 6 h. Purification by FC (n-hexane/ethyl acetate 6:1) afforded the desired compound as colorless solid (130 mg, 96%); MS (ESI): 368 (M+H)⁺; ¹H NMR (CD₃COCD₃): 3.16 (br s, 3H), 3.80 (s, 3H), 3.86 (s, 3H), 4.79 (br s, 2H), 6.86-6.88 (m, 1H), 6.91-6.95 (m, 3H), 7.23 (t, J=2.0 Hz, 1H), 7.25-7.28 (m, 1H), 7.30 (t, J=8.0 Hz, 1H), 7.35 (t, J=8.0 Hz, 1H), 7.41 (br s, 2H); ¹³C NMR (CD₃COCD₃): 55.5, 55.7, 112.1, 113.5, 114.9, 119.1, 124.3, 130.6, 131.1, 135.7, 138.7, 140.1, 148.1, 161.1, 161.3.

### 1.24. N-(3-Hydroxybenzyl)-5-(3-hydroxyphenyl)-N-methylthiophene-2-carboxamide (24)

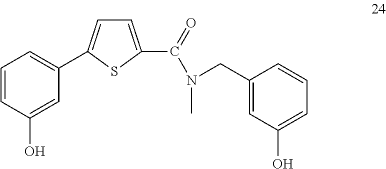 [SMILES: CN(Cc1cccc(O)c1)C(=O)c1ccc(-c2cccc(O)c2)s1]

The title compound was prepared by reaction of N-(3-methoxybenzyl)-5-(3-methoxyphenyl)-N-methylthiophene-2-carboxamide (23) (70 mg, 0.19 mmol) with boron trifluoride dimethyl sulfide complex (0.24 ml, 2.28 mmol) according to method C for 4 h. Purification by FC(CH₂Cl₂/CH₃OH 100:1→50:1) yielded the title compound as colorless solid (59 mg, 91%); MS (ESI): 340 (M+H)⁺; ¹H NMR (CD₃COCD₃): 3.15 (br s, 3H), 4.76 (br s, 2H), 6.77-6.79 (m, 1H), 6.80-6.83 (m, 2H), 6.85 (ddd, J=8.0, 2.5, 0.9 Hz, 1H), 7.15-7.18 (m, 2H), 7.21 (t, J=8.0 Hz, 1H), 7.26 (t, J=7.9 Hz, 1H), 7.34 (d, J=3.5 Hz, 1H), 7.39 (br s, 1H), 8.35 (s, 1H), 8.54 (s, 1H); ¹³C NMR (CD₃COCD₃): 113.5, 115.2, 116.4, 118.0, 124.0, 130.7, 131.1, 135.7, 138.4, 140.0, 148.4, 158.8, 158.9.

### 1.25. N-(3-Methoxybenzyl)-5-(4-methoxyphenyl)-N-methylthiophene-2-carboxamide (25)

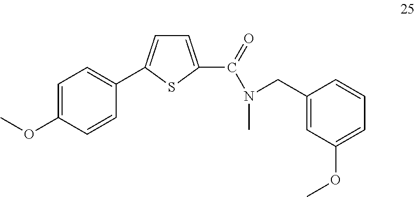 [SMILES: COc1ccc(-c2ccc(C(=O)N(C)Cc3cccc(OC)c3)s2)cc1]

The title compound was prepared by reaction of 5-bromo-N-(3-methoxybenzyl)-N-methylthiophene-2-carboxamide (23a) (130 mg, 0.38 mmol) and 4-methoxyphenylboronic acid (70 mg, 0.46 mmol) with tetrakis(triphenylphosphine) palladium (44 mg, 0.04 mmol) as catalyst according to method B for 6 h. Purification by FC (n-hexane/ethyl acetate 6:1) afforded the desired compound as colorless solid (138 mg, 98%); MS (ESI): 368 (M+H)⁺; ¹H NMR (CD₃COCD₃): 3.17 (br s, 3H), 3.80 (s, 3H), 3.84 (s, 3H), 4.79 (br s, 2H), 6.87 (d, J=8.0 Hz, 1H), 6.91-6.93 (m, 2H), 7.00 (d, J=8.8 Hz, 2H), 7.27-7.32 (m, 2H), 7.38 (br s, 1H), 7.63 (d, J=8.8 Hz, 2H); ¹³C NMR (CD₃COCD₃): 55.5, 55.7, 113.5, 115.4, 122.9, 127.1, 128.1, 130.6, 137.6, 140.2, 148.5, 161.0, 161.1.

### 1.26. N-(3-Hydroxybenzyl)-5-(4-hydroxyphenyl)-N-methylthiophene-2-carboxamide (26)

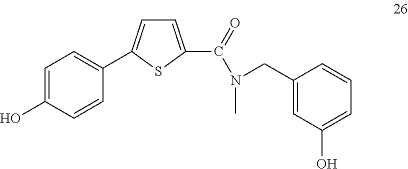 [SMILES: CN(Cc1cccc(O)c1)C(=O)c1ccc(-c2ccc(O)cc2)s1]

The title compound was prepared by reaction of N-(3-methoxybenzyl)-5-(4-methoxyphenyl)-N-methylthiophene-2-carboxamide (25) (100 mg, 0.27 mmol) with borontrifluoride dimethyl sulfide complex (0.34 ml, 3.24 mmol) according to method C for 4 h. Purification by FC(CH₂Cl₂/CH₃OH 100:1→50:1) yielded the title compound as colorless solid (85 mg, 92%); MS (ESI): 340 (M+H)⁺; ¹H NMR (CD₃COCD₃): 3.15 (br s, 3H), 4.75 (br s, 2H), 6.76-6.80 (m, 3H), 6.90 (d, J=8.5 Hz, 2H), 7.19-7.23 (m, 2H), 7.36 (br s, 1H), 7.54 (d, J=8.5 Hz, 2H), 8.40 (s, 1H), 8.70 (s, 1H); ¹³C NMR (CD₃COCD₃): 115.2, 116.8, 122.5, 126.1, 128.2, 130.6, 137.1, 140.1, 149.0, 158.8, 158.9.

### 1.27. N-(3-Methoxybenzyl)-5-(2-methoxyphenyl)-N-methylthiophene-2-carboxamide (27)

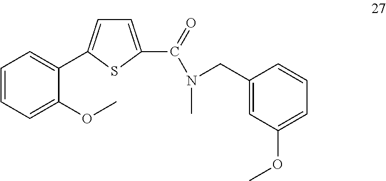 [SMILES: COc1cccc(CN(C)C(=O)c2ccc(-c3ccccc3OC)s2)c1]

The title compound was prepared by reaction of 5-bromo-N-(3-methoxybenzyl)-N-methylthiophene-2-carboxamide (23a) (90 mg, 0.26 mmol) and 2-methoxyphenyl boronic acid (48 mg, 0.32 mmol) with tetrakis(triphenylphosphine) palladium (30 mg, 0.026 mmol) as catalyst according to method B for 4 h. Purification by FC (n-hexane/ethyl acetate 6:1) afforded the desired compound as colorless solid (92 mg, 93%); MS (ESI): 368 (M+H)⁺; ¹H NMR (CD₃COCD₃): 3.16 (br s, 3H), 3.80 (s, 3H), 3.97 (s, 3H), 4.80 (br s, 2H), 6.87 (dd, J=8.0, 2.0 Hz, 1H), 6.92-6.94 (m, 2H), 7.03 (td, J=7.0, 1.0 Hz, 1H), 7.15 (dd, J=8.0, 1.0 Hz, 1H), 7.29-7.36 (m, 2H), 7.39 (br s, 1H), 7.52 (d, J=4.0 Hz, 1H), 7.76 (dd, J=8.0, 1.5 Hz, 1H); ¹³C NMR (CD₃COCD₃): 55.5, 56.0, 112.9, 113.5, 121.8, 123.1, 125.5, 128.8, 129.5, 130.2, 130.6, 132.0, 138.6, 140.3, 143.5, 156.7, 161.1.

### 1.28. N-(3-Hydroxybenzyl)-5-(2-hydroxyphenyl)-N-methylthiophene-2-carboxamide (28)

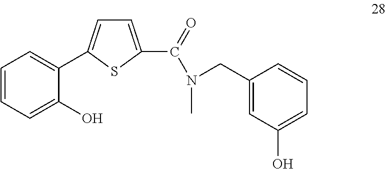 [SMILES: CN(Cc1cccc(O)c1)C(=O)c1ccc(-c2ccccc2O)s1]

The title compound was prepared by reaction of N-(3-methoxybenzyl)-5-(2-methoxyphenyl)-N-methylthiophene-2-carboxamide (27) (48 mg, 0.13 mmol) with borontrifluoride dimethyl sulfide complex (0.16 ml, 1.56 mmol) according to method C for 14 h. Purification by FC(CH₂Cl₂/CH₃OH 100:1→50:1) yielded the title compound as colorless solid (40 mg, 90%); MS (ESI): 340 (M+H)⁺; ¹H NMR (CD₃COCD₃): 3.15 (br s, 3H), 4.76 (br s, 2H), 6.77 (dd, J=8.0, 2.0 Hz, 1H), 6.81-6.84 (m, 2H), 6.92 (td, J=7.6, 0.9 Hz, 1H), 7.04 (dd, J=8.0, 0.9 Hz, 1H), 7.16-7.22 (m, 2H), 7.39 (br s, 1H), 7.55 (d, J=3.4 Hz, 1H), 7.70 (dd, J=8.0, 0.9 Hz, 1H), 8.40 (s, 1H), 9.34 (s, 1H); ¹³C NMR (CD₃COCD₃): 115.2, 117.3, 120.9, 121.4, 125.3, 128.9, 130.0, 130.6, 138.1, 140.1, 144.5, 154.6, 158.8.

### 1.29. N-(3-Methoxybenzyl)-N-methyl-5-phenylthiophene-2-carboxamide (29)

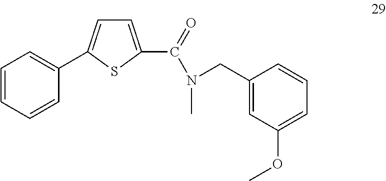 [SMILES: COc1cccc(CN(C)C(=O)c2ccc(-c3ccccc3)s2)c1]

The title compound was prepared by reaction of 5-bromo-N-(3-methoxybenzyl)-N-methylthiophene-2-carboxamide (23a) (90 mg, 0.27 mmol) and phenylboronic acid (43 mg, 0.35 mmol) with tetrakis(triphenylphosphine) palladium (31 mg, 0.027 mmol) as catalyst according to method B for 14 h. Purification by FC (n-hexane/ethyl acetate 6:1) afforded the desired compound as colorless solid (87 mg, 96%); MS (ESI): 338 (M+H)⁺; ¹H NMR (CD₃COCD₃): 3.17 (br s, 3H), 3.80 (s, 3H), 4.80 (br s, 2H), 6.88 (ddd, J=8.2, 2.5, 0.9 Hz, 1H), 6.92 (s, 1H), 6.93 (d, J=7.9 Hz, 1H), 7.32 (t, J=8.2 Hz, 1H), 7.36 (t, J=7.6 Hz, 1H), 7.41-7.46 (m, 4H), 7.71 (d, J=7.6 Hz, 2H); ¹³C NMR (CD₃COCD₃): 55.5, 113.5, 124.1, 126.7, 129.2, 130.0, 130.6, 134.5, 140.1, 148.3, 161.1.

### 1.30. N-(3-Hydroxybenzyl)-N-methyl-5-phenylthiophene-2-carboxamide (30)

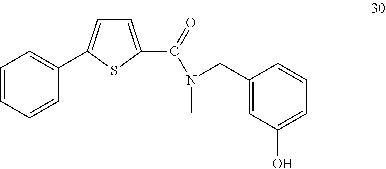 [SMILES: CN(Cc1cccc(O)c1)C(=O)c1ccc(-c2ccccc2)s1]

The title compound was prepared by reaction of N-(3-methoxybenzyl)-N-methyl-5-phenylthiophene-2-carboxamide (29) with borontrifluoride dimethyl sulfide complex (0.13 ml, 1.20 mmol) according to method C for 6 h. Purification by FC(CH₂Cl₂/CH₃OH 100:1→50:1) yielded the title compound as colorless solid (63 mg, 98%); MS (ESI): 346 (M+CH₃CN); ¹H NMR (CD₃COCD₃): 3.16 (br s, 3H), 4.76 (br s, 2H), 6.78 (ddd, J=8.2, 2.5, 0.9 Hz, 1H), 6.81-6.83 (m, 2H), 7.22 (t, J=7.8 Hz, 1H), 7.36 (tt, J=8.2, 1.3 Hz, 1H), 7.40-7.46 (m, 4H), 7.71 (d, J=7.6 Hz, 2H), 8.37 (s, 1H); ¹³C NMR (CD₃COCD₃): 115.2, 124.0, 126.7, 129.2, 130.0, 130.7, 134.5, 138.7, 140.0, 148.3, 158.8.

### 1.31. 5-(4-Cyanophenyl)-N-(3-methoxybenzyl)-N-methylthiophene-2-carboxamide (31)

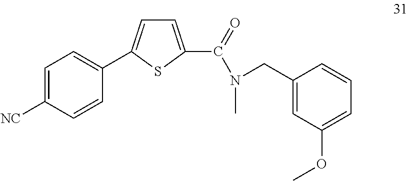 [SMILES: COc1cccc(CN(C)C(=O)c2ccc(-c3ccc(C#N)cc3)s2)c1]

The title compound was prepared by reaction of 5-bromo-N-(3-methoxybenzyl)-N-methylthiophene-2-carboxamide (23a) (80 mg, 0.24 mmol) and 4-cyanophenylboronic acid (42 mg, 0.29 mmol) with tetrakis(triphenylphosphine) palladium (28 mg, 0.024 mmol) as catalyst according to method B for 14 h. Purification by FC (n-hexane/ethyl acetate 6:1) afforded the desired compound as colorless solid (80 mg, 92%); MS (ESI): 363 (M+H)⁺; ¹H NMR (CD₃COCD₃): 3.18 (br s, 3H), 3.80 (s, 3H), 4.80 (br s, 2H), 6.87-6.89 (m, 1H), 6.91-6.93 (m, 2H), 7.31 (t, J=7.9 Hz, 1H), 7.46 (s, 1H), 7.60 (d, J=3.3 Hz, 1H), 7.84 (d, J=8.8 Hz, 2H), 7.92 (d, J=8.2 Hz, 2H); ¹³C NMR (CD₃COCD₃): 55.5, 112.3, 113.6, 119.1, 126.3, 127.3, 130.7, 133.9, 138.7, 140.0, 145.7, 161.2.

### 1.32. 5-(4-Cyanophenyl)-N-(3-hydroxybenzyl)-N-methylthiophene-2-carboxamide (32)

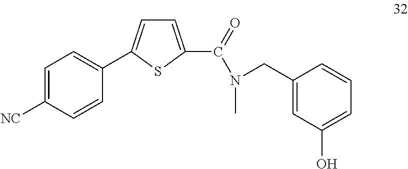 [SMILES: CN(Cc1cccc(O)c1)C(=O)c1ccc(-c2ccc(C#N)cc2)s1]

The title compound was prepared by reaction of 5-(4-cyanophenyl)-N-(3-methoxybenzyl)-N-methylthiophene-2-carboxamide (31) (60 mg, 0.17 mmol) with borontrifluoride dimethyl sulfide complex (0.11 ml, 1.02 mmol) according to method C for 14 h. Purification by FC(CH₂Cl₂/CH₃OH 100:1→50:1) yielded the title compound as yellowish solid (55 mg, 95%); MS (ESI): 349 (M+H)⁺; ¹H NMR (CD₃COCD₃): 3.16 (br s, 3H), 4.76 (br s, 2H), 6.77-6.82 (m, 3H), 7.21 (t, J=7.7 Hz, 1H), 7.46 (br s, 1H), 7.60 (d, J=2.8 Hz, 1H), 7.83 (d, J=8.5 Hz, 2H), 7.92 (d, J=8.5 Hz, 2H), 8.37 (s, 1H); ¹³C NMR (CD₃COCD₃): 112.3, 115.2, 115.3, 118.8, 119.1, 126.3, 127.3, 130.7, 133.9, 138.7, 139.9, 145.7, 158.8.

### 1.33. N-Benzyl-5-(3-methoxyphenyl)-N-methylthiophene-2-carboxamide (33)

### N-Benzyl-5-bromo-N-methylthiophene-2-carboxamide (33a)

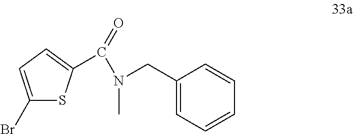 [SMILES: CN(Cc1ccccc1)C(=O)c1ccc(Br)s1]

The title compound was prepared by reaction of 5-bromothiophene-2-carbonyl chloride (225 mg, 1 mmol) and N-methylbenzylamine (121 mg, 1 mmol) in presence of triethylamine (0.16 ml, 1.15 mmol) according to method A. Purification by FC (n-hexane/ethyl acetate 6:1) afforded the desired compound as colorless oil (298 mg, 96%). MS (ESI): 311 (M+H)⁺; ¹H NMR (CD₃COCD₃): 3.14 (br s, 3H), 4.78 (br s, 2H), 7.14 (d, J=3.8 Hz, 1H), 7.25-7.40 (m, 6H); ¹³C NMR (CD₃COCD₃): 117.1, 128.3, 129.6, 131.4, 138.2, 141.9.

### N-Benzyl-5-(3-methoxyphenyl)-N-methylthiophene-2-carboxamide (33)

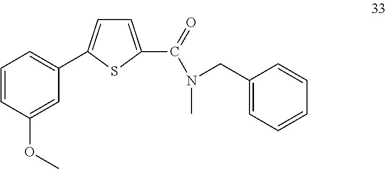 [SMILES: COc1cccc(-c2ccc(C(=O)N(C)Cc3ccccc3)s2)c1]

The title compound was prepared by reaction of by reaction of N-benzyl-5-bromo-N-methylthiophene-2-carboxamide (33a) (80 mg, 0.26 mmol) and 3-methoxyphenylboronic acid (47 mg, 0.31 mmol) with tetrakis(triphenylphosphine) palladium (30 mg, 0.026 mmol) as catalyst according to method B for 4 h. Purification by FC (n-hexane/ethyl acetate 6:1) afforded the desired compound as colorless solid (75 mg, 86%); MS (ESI): 338 (M+H)⁺; ¹H NMR (CD₃COCD₃): 3.17 (br s, 3H), 3.86 (s, 3H), 4.82 (br s, 2H), 6.94 (ddd, J=8.2, 2.5, 0.9 Hz, 1H), 7.23 (t, J=2.0 Hz, 1H), 7.27 (ddd, J=7.9, 1.6, 0.9 Hz, 1H), 7.29-7.32 (m, 1H), 7.34-7.41 (m, 7H); ¹³C NMR (CD₃COCD₃): 55.7, 112.1, 114.9, 119.1, 124.3, 128.2, 129.6, 131.1, 135.7, 138.5, 148.1, 161.3.

### 1.34. N-Benzyl-5-(3-hydroxyphenyl)-N-methylthiophene-2-carboxamide (34)

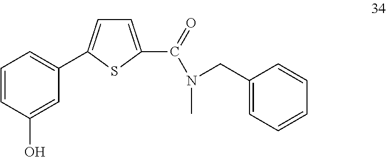 [SMILES: CN(Cc1ccccc1)C(=O)c1ccc(-c2cccc(O)c2)s1]

The title compound was prepared by reaction of N-benzyl-5-(3-methoxyphenyl)-N-methylthiophene-2-carboxamide (33) (65 mg, 0.19 mmol) with borontrifluoride dimethyl sulfide complex (0.12 ml, 1.14 mmol) according to method C for 4 h. Purification by FC(CH₂Cl₂/CH₃OH 100:1) yielded the title compound as colorless solid (55 mg, 88%). MS (ESI): 324 (M+H)⁺; ¹H NMR (CD₃COCD₃): 3.17 (br s, 3H), 4.82 (br s, 2H), 6.85 (ddd, J=8.2, 2.2, 0.9 Hz, 1H), 7.16-7.18 (m, 2H), 7.26 (t, J=7.8 Hz, 1H), 7.29-7.33 (m, 1H), 7.34-7.41 (m, 6H), 8.55 (s, 1H); ¹³C NMR (CD₃COCD₃): 113.5, 116.4, 118.0, 124.0, 128.2, 129.6, 131.1, 135.7, 138.5, 148.4, 158.9.

### 1.35. N-Benzyl-5-(4-methoxyphenyl)-N-methylthiophene-2-carboxamide (35)

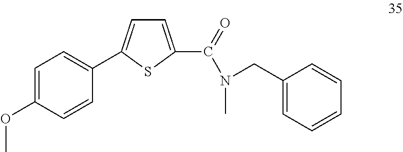 [SMILES: COc1ccc(-c2ccc(C(=O)N(C)Cc3ccccc3)s2)cc1]

The title compound was prepared by reaction of N-benzyl-5-bromo-N-methylthiophene-2-carboxamide (33a) (72 mg, 0.23 mmol) and 4-methoxyphenylboronic acid (42 mg, 0.27 mmol) with tetrakis(triphenylphosphine) palladium (27 mg, 0.023 mmol) as catalyst according to method B for 5 h. Purification by FC (n-hexane/ethyl acetate 6:1) afforded the desired compound as colorless solid (75 mg, 96%); MS (ESI): 338 (M+H)⁺; ¹H NMR (CD₃COCD₃): 3.16 (br s, 3H), 3.84 (s, 3H), 4.82 (br s, 2H), 7.00 (d, J=8.8 Hz, 2H); 7.27 (d, J=3.8 Hz, 1H), 7.29-7.32 (m, 1H), 7.35-7.41 (m, 5H), 7.63 (d, J=8.8 Hz, 2H); ¹³C NMR (CD₃COCD₃): 55.7, 115.4, 122.9, 127.1, 128.1, 128.2, 129.6, 137.6, 138.6, 148.5, 161.0.

### 1.36. N-Benzyl-5-(4-hydroxyphenyl)-N-methylthiophene-2-carboxamide (36)

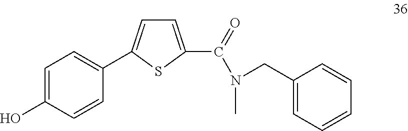 [SMILES: CN(Cc1ccccc1)C(=O)c1ccc(-c2ccc(O)cc2)s1]

The title compound was prepared by reaction of N-benzyl-5-(4-methoxyphenyl)-N-methylthiophene-2-carboxamide (35) (56 mg, 0.17 mmol) with borontrifluoride dimethyl sulfide complex (0.11 ml, 1.02 mmol) according to method C for 6 h. Purification by FC(CH₂Cl₂/CH₃OH 100:1) yielded the title compound as colorless solid (49 mg, 91%); MS: 324 (M+H)⁺; ¹H NMR (CD₃COCD₃): 3.16 (br s, 3H), 4.82 (br s, 2H), 6.91 (d, J=8.8 Hz, 2H), 7.23 (d, J=3.8 Hz, 1H), 7.28-7.32 (m, 1H), 7.34-7.40 (m, 5H), 7.55 (d, J=8.8 Hz, 2H), 8.64 (s, 1H); ¹³C NMR (CD₃COCD₃): 116.8, 122.5, 126.1, 128.2, 129.6, 137.2, 138.6, 149.0, 158.9.

### 1.37. N-Benzyl-5-(2-methoxyphenyl)-N-methylthiophene-2-carboxamide (37)

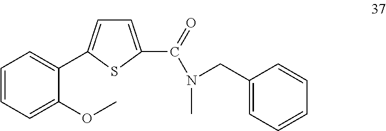 [SMILES: COc1ccccc1-c1ccc(C(=O)N(C)Cc2ccccc2)s1]

The title compound was prepared by reaction of N-benzyl-5-bromo-N-methylthiophene-2-carboxamide (33a) (80 mg, 0.26 mmol) and 2-methoxyphenylboronic acid (47 mg, 0.31 mmol) with tetrakis(triphenylphosphine) palladium (30 mg, 0.026 mmol) as catalyst according to method B for 14 h. Purification by FC (n-hexane/ethyl acetate 6:1) afforded the desired compound as colorless solid (76 mg, 87%); MS (ESI): 338 (M+H)⁺; ¹H NMR (CD₃COCD₃): 3.16 (br s, 3H), 3.97 (s, 3H), 4.82 (br s, 2H), 7.03 (td, J=7.6, 1.3 Hz, 1H), 7.15 (dd, J=8.2, 1.3 Hz, 1H), 7.29-7.41 (m. 7H), 7.52 (d, J=4.0 Hz, 1H), 7.76 (dd, J=7.8, 1.6 Hz, 1H); ¹³C NMR (CD₃COCD₃): 56.0, 112.9, 121.8, 123.1, 125.5, 128.1, 128.8, 129.5, 130.2, 132.0, 138.6, 143.5, 156.7.

### 1.38. N-Benzyl-5-(2-hydroxyphenyl)-N-methylthiophene-2-carboxamide (38)

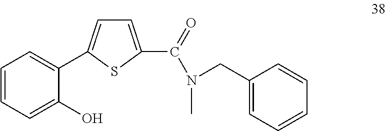 [SMILES: CN(Cc1ccccc1)C(=O)c1ccc(-c2ccccc2O)s1]

The title compound was prepared by reaction of N-benzyl-5-(2-methoxyphenyl)-N-methylthiophene-2-carboxamide (37) (55 mg, 0.16 mmol) with borontrifluoride dimethyl sulfide complex (0.10 ml, 0.96 mmol) according to method C for 14 h. Purification by FC(CH₂Cl₂/CH₃OH 100:1) yielded the title compound as colorless solid (49 mg, 93%); MS: 324 (M+H)⁺; ¹H NMR (CD₃COCD₃): 3.16 (br s, 3H), 4.83 (br s, 2H), 6.93 (ddd, J=8.5, 7.3, 1.3 Hz, 1H), 7.03 (dd, J=7.8, 1.3 Hz, 1H), 7.18 (ddd, J=8.5, 7.3, 1.3 Hz, 1H), 7.28-7.32 (m, 1H), 7.35-7.41 (m, 5H), 7.56 (d, J=4.0 Hz, 1H), 7.70 (dd, J=7.8, 1.6 Hz, 1H), 9.23 (s, 1H); ¹³C NMR (CD₃COCD₃): 117.3, 121.0, 121.5, 125.4, 128.1, 129.0, 129.6, 130.0, 138.2, 138.7, 154.6.

### 1.39. N-(3-Hydroxybenzyl)-5-(4-methoxyphenyl)-N-methylthiophene-2-carboxamide (39)

### 5-Bromo-N-(3-hydroxybenzyl)-N-methylthiophene-2-carboxamide (39a)

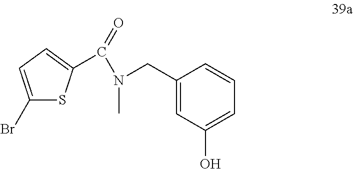 [SMILES: CN(Cc1cccc(O)c1)C(=O)c1ccc(Br)s1]

The title compound was prepared by reaction of 23a (340 mg, 1 mmol) and borontrifluoride dimethyl sulfide complex (0.63 ml, 6 mmol) according to method C for 5 h. Purification by FC(CH₂Cl₂/CH₃OH 100:1) afforded the desired compound as colorless solid (300 mg, 93%); ¹H NMR (CD₃COCD₃): 3.13 (br s, 3H), 4.71 (br s, 2H), 6.76-6.79 (m, 3H), 7.13-7.14 (m, 1H), 7.20 (t, J=7.7 Hz, 1H), 7.25 (br s, 1H), 8.69 (s, 1H).

### N-(3-Hydroxybenzyl)-5-(4-methoxyphenyl)-N-methylthiophene-2-carboxamide (39)

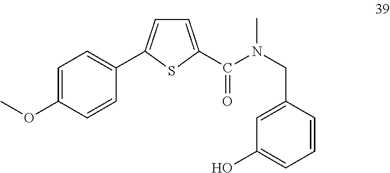 [SMILES: COc1ccc(-c2ccc(C(=O)N(C)Cc3cccc(O)c3)s2)cc1]

The title compound was prepared by reaction of 5-bromo-N-(3-hydroxybenzyl)-N-methylthiophene-2-carboxamide (39a) (49 mg, 0.15 mmol) and 4-methoxyphenylboronic acid (27 mg, 0.18 mmol) with tetrakis(triphenylphosphine) palladium (17 mg, 0.015 mmol) as catalyst according to method B for 6 h. Purification by FC (n-hexane/ethyl acetate 6:1) afforded the desired compound as colorless solid (48 mg, 90%); MS (ESI): 354 (M+H)⁺; ¹H NMR (CD₃COCD₃): 3.15 (br s, 3H), 3.84 (s, 3H), 4.75 (br s, 2H), 6.77 (dd, J=8.0, 2.2 Hz, 1H), 6.80-6.83 (m, 2H), 7.00 (d, J=8.8 Hz, 2H), 7.21 (t, J=8.0 Hz, 1H), 7.27 (d, J=3.5 Hz, 1H), 7.31 (br s, 1H), 7.63 (d, J=8.8 Hz, 2H), 8.35 (s, 1H); ¹³C NMR (CD₃COCD₃): 55.7, 115.2, 115.4, 122.9, 127.1, 128.1, 129.6, 130.6, 132.0, 133.4, 140.1, 143.9, 158.8.

### 1.40. 5-(3-(Dimethylamino)phenyl)-N-(3-hydroxybenzyl)-N-methylthiophene-2-carboxamide (40)

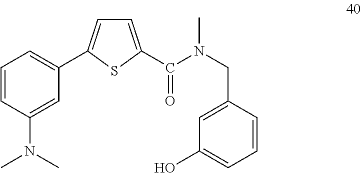 [SMILES: CN(Cc1cccc(O)c1)C(=O)c1ccc(-c2cccc(N(C)C)c2)s1]

The title compound was prepared by reaction of 5-bromo-N-(3-hydroxybenzyl)-N-methylthiophene-2-carboxamide (39a) (49 mg, 0.15 mmol) and N,N-dimethyl-3-aminophenylboronic acid (30 mg, 0.18 mmol) with tetrakis(triphenylphosphine) palladium (17 mg, 0.015 mmol) as catalyst according to method B for 14 h. Purification by FC (n-hexane/ethyl acetate 5:1→3:1) afforded the desired compound as yellowish solid (45 mg, 82%); MS (ESI): 367 (M+H)⁺; ¹H NMR (CD₃COCD₃): 3.00 (s, 6H), 3.15 (br s, 3H), 4.75 (br s, 2H), 6.75-6.82 (m, 4H), 6.98 (d, J=4.0 Hz, 1H), 7.00 (s, 1H), 7.19-7.26 (m, 2H), 7.36 (s, 1H), 7.39 (br s, 1H), 8.36 (s, 1H); ¹³C NMR (CD₃COCD₃): 40.5, 110.5, 113.5, 114.9, 115.2, 123.7, 130.5, 135.0, 138.1, 140.1, 149.6, 152.1, 157.8, 158.8.

### 1.41. 5-(3-Fluorophenyl)-N-(3-hydroxybenzyl)-N-methylthiophene-2-carboxamide (41)

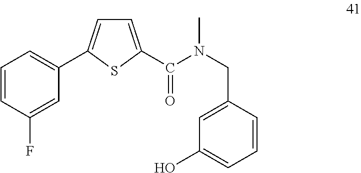 [SMILES: CN(Cc1cccc(O)c1)C(=O)c1ccc(-c2cccc(F)c2)s1]

The title compound was prepared by reaction of 5-bromo-N-(3-hydroxybenzyl)-N-methylthiophene-2-carboxamide (39a) (49 mg, 0.15 mmol) and 3-fluorophenylboronic acid (25 mg, 0.18 mmol) with tetrakis(triphenylphosphine) palladium (17 mg, 0.015 mmol) as catalyst according to method B for 14 h. Purification by FC (n-hexane/ethyl acetate 6:13:1) afforded the desired compound as colorless solid (40 mg, 78%); MS (ESI): 342 (M+H)⁺; ¹H NMR (CD₃COCD₃): 3.16 (br s, 3H), 4.76 (br s, 2H), 6.77-6-82 (m, 3H), 7.11-7.15 (m, 1H), 7.21 (t, J=7.7 Hz, 1H), 7.42 (br s, 1H), 7.46-7.51 (m, 3H), 7.54 (d, J=8.2 Hz, 1H), 8.35 (s, 1H); ¹³C NMR (CD₃COCD₃): 113.2, 115.3, 115.7, 115.9, 122.7, 125.1, 130.7, 131.9, 132.0, 136.8, 139.2, 140.1, 144.4, 146.5, 148.3, 150.9, 158.8, 160.9, 163.1, 165.1.

### 1.42. 5-(4-Fluorophenyl)-N-(3-hydroxybenzyl)-N-methylthiophene-2-carboxamide (42)

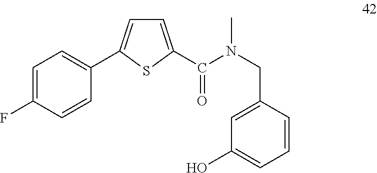 [SMILES: CN(Cc1cccc(O)c1)C(=O)c1ccc(-c2ccc(F)cc2)s1]

The title compound was prepared by reaction of 5-bromo-N-(3-hydroxybenzyl)-N-methylthiophene-2-carboxamide (39a) (49 mg, 0.15 mmol) and 4-fluorophenylboronic acid (25 mg, 0.18 mmol) with tetrakis(triphenylphosphine) palladium (17 mg, 0.015 mmol) as catalyst according to method B for 14 h. Purification by FC (n-hexane/ethyl acetate 5:1→3:1) afforded the desired compound as colorless solid (43 mg, 84%); MS (ESI): 342 (M+H)⁺; ¹H NMR (CD₃COCD₃): 3.16 (br s, 3H), 4.75 (br s, 2H), 6.77-6.82 (m, 3H), 7.19-7.24 (m, 3H), 7.37 (d, J=3.7 Hz, 1H), 7.40 (br s, 1H), 7.73-7.76 (m, 2H), 8.35 (br s, 1H); ¹³C NMR (CD₃COCD₃): 115.3, 116.8, 116.9, 124.3, 127.8, 128.7, 128.8, 130.6, 131.0, 138.9, 140.0, 147.1, 158.8, 162.7, 164.2, 164.6.

### 1.43. 5-(2-Fluoro-3-methoxyphenyl)-N-(3-hydroxybenzyl)-N-methylthiophene-2-carboxamide (43)

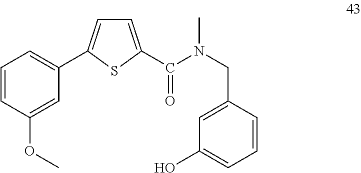 [SMILES: COc1cccc(-c2ccc(C(=O)N(C)Cc3cccc(O)c3)s2)c1]

The title compound was prepared by reaction of 5-bromo-N-(3-hydroxybenzyl)-N-methylthiophene-2-carboxamide (39a) (49 mg, 0.15 mmol) and 2-fluoro-3-methoxyphenylboronic acid (31 mg, 0.18 mmol) with tetrakis(triphenylphosphine) palladium (17 mg, 0.015 mmol) as catalyst according to method B for 14 h. Purification by FC (n-hexane/ethyl acetate 5:1→3:1) afforded the desired compound as colorless solid (40 mg, 72%); MS (ESI): 372 (M+H)⁺; ¹H NMR (CD₃COCD₃): 3.16 (br s, 3H), 3.93 (s, 3H), 4.76 (br s, 2H), 6.77-6.83 (m, 3H), 7.13-7.23 (m, 3H), 7.31 (td, J=7.8, 1.6 Hz, 1H), 7.45 (br s, 1H), 7.48 (br s, 1H), 8.35 (s, 1H); ¹³C NMR (CD₃COCD₃): 56.7, 114.0, 114.1, 115.3, 120.5, 120.6, 122.7, 122.8, 125.6, 127.2, 130.7, 139.9, 140.7, 148.9, 149.6, 150.9, 158.8.

### 1.44. 5-(2-Fluoro-3-hydroxyphenyl)-N-(3-hydroxybenzyl)-N-methylthiophene-2-carboxamide (44)

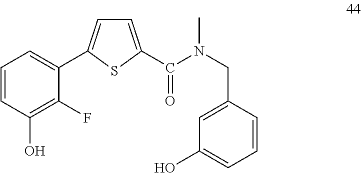 [SMILES: CN(Cc1cccc(O)c1)C(=O)c1ccc(-c2cccc(O)c2F)s1]

The title compound was prepared by reaction of 5-(2-fluoro-3-methoxyphenyl)-N-(3-hydroxybenzyl)-N-methylthiophene-2-carboxamide (43) (30 mg, 0.08 mmol) with borontrifluoride dimethyl sulfide complex (0.05 ml, 0.48 mmol) according to method C for 14 h. Purification by FC(CH₂Cl₂/CH₃OH 100:1) yielded the title compound as colorless solid (25 mg, 87%); MS (ESI): 358 (M+H)⁺; ¹H NMR (CD₃COCD₃): 3.16 (br s, 3H), 4.76 (br s, 2H), 6.77-6.99 (m, 3H), 7.01 (td, J=8.0, 1.6 Hz, 1H), 7.08 (td, J=8.0, 0.9 Hz, 1H), 7.21 (t, J=8.0 Hz, 2H), 7.44 (br s, 1H), 7.46 (s, 1H), 8.36 (s, 1H), 8.86 (d, J=1.6 Hz, 1H); ¹³C NMR (CD₃COCD₃): 115.3, 118.4, 119.7, 122.9, 125.6, 126.6, 127.0, 127.1, 130.6, 139.6, 140.0, 141.1, 146.7, 148.3, 150.3, 158.8.

### 1.45. N-(3-Hydroxybenzyl)-5-(3-methoxyphenyl)-N-methylthiophene-2-carboxamide (45)

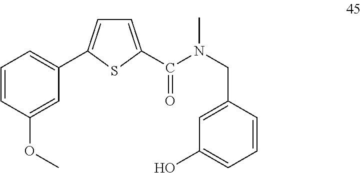 [SMILES: COc1cccc(-c2ccc(C(=O)N(C)Cc3cccc(O)c3)s2)c1]

The title compound was prepared by reaction of 5-bromo-N-(3-hydroxybenzyl)-N-methylthiophene-2-carboxamide (39a) (33 mg, 1 mmol) and 3-methoxyphenylboronic acid (18 mg, 0.12 mmol) with tetrakis(triphenylphosphine) palladium (12 mg, 0.01 mmol) as catalyst according to method B for 6 h. Purification by FC (n-hexane/ethyl acetate 6:1-3:1) afforded the desired compound as colorless solid (25 mg, 71%). MS (ESI): 354 (M+H)⁺; ¹H NMR (CD₃COCD₃): 3.16 (br s, 3H), 3.86 (s, 3H), 4.76 (br s, 2H), 6.77-6.82 (m, 3H), 6.94 (ddd, J=8.0, 2.5, 0.9 Hz, 1H), 7.21 (t, J=8.0 Hz, 1H), 7.23 (t, J=2.0 Hz, 1H), 7.27 (d, J=8.0 Hz, 1H), 7.35 (t, J=8.0 Hz, 1H), 7.41 (br, s, 2H), 8.36 (s, 1H); ¹³C NMR (CD₃COCD₃): 55.7, 112.1, 114.9, 115.3, 119.1, 130.6, 131.1, 135.7, 140.0, 148.1, 158.8, 161.2.

### 1.46. N-(3-Hydroxybenzyl)-N-methyl-5-m-tolylthiophene-2-carboxamide (46)

### N-(3-Methoxybenzyl)-N-methyl-5-m-tolylthiophene-2-carboxamide (46a)

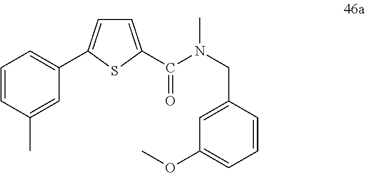 [SMILES: COc1cccc(CN(C)C(=O)c2ccc(-c3cccc(C)c3)s2)c1]

The title compound was prepared by reaction of 5-bromo-N-(3-methoxybenzyl)-N-methylthiophene-2-carboxamide (23a) (53 mg, 0.15 mol) and 3-methylphenylboronic acid (24 mg, 0.18 mmol) with tetrakis(triphenylphosphine) palladium (17 mg, 0.015 mmol) as catalyst according to method B for 6 h. Purification by FC (n-hexane/ethyl acetate 6:1) afforded the desired compound as colorless oil (50 mg, 95%); MS (ESI): 352 (M+H)⁺; ¹H NMR (CD₃COCD₃): 2.38 (s, 3H), 3.17 (br s, 3H), 3.80 (s, 3H), 4.80 (br s, 2H), 6.86-6.89 (m, 1H), 6.91-6.93 (m, 2H), 7.19 (d, J=7.6 Hz, 1H), 7.29-7.33 (m, 2H), 7.38-7.42 (m, 2H), 7.49 (d, J=7.6 Hz, 1H), 7.53 (s, 1H); ¹³C NMR (CD₃COCD₃): 21.3, 55.5, 113.5, 123.8, 123.9, 127.3, 129.9, 130.0, 130.6, 134.4, 138.5, 139.7, 140.1, 148.5, 161.1.

### N-(3-Hydroxybenzyl)-N-methyl-5-m-tolylthiophene-2-carboxamide (46)

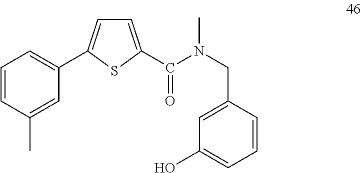 [SMILES: Cc1cccc(-c2ccc(C(=O)N(C)Cc3cccc(O)c3)s2)c1]

The title compound was prepared by reaction of N-(3-methoxybenzyl)-N-methyl-5-m-tolylthiophene-2-carboxamide (46a) (35 mg, 0.1 mmol) with borontrifluoride dimethyl sulfide complex (0.06 ml, 0.6 mmol) according to method C for 4 h. Purification by FC(CH₂Cl₂/CH₃OH 100:1) yielded the title compound as colorless solid (30 mg, 89%); MS (ESI): 338 (M+H)⁺; ¹H NMR (CD₃COCD₃): 2.37 (s, 3H), 3.15 (br s, 3H), 4.75 (br s, 2H), 6.78 (ddd, J=8.2, 2.5, 0.9 Hz, 1H), 6.80-6.83 (m, 2H), 7.17-7.22 (m, 2H), 7.32 (t, J=7.8 Hz, 1H), 7.37-7.43 (m, 2H), 7.49 (d, J=7.8 Hz, 1H), 7.53 (s, 1H), 8.46 (s, 1H); ¹³C NMR (CD₃COCD₃): 21.3, 115.2, 123.8, 123.9, 127.3, 129.9, 130.0, 130.7, 134.4, 138.5, 139.7, 140.0, 148.5, 158.8.

### 1.47. N,5-bis(3-Methoxyphenyl)-N-methylthiophene-2-carboxamide (47)

### 5-Bromo-N-(3-methoxyphenyl)-N-methylthiophene-2-carboxamide (47a)

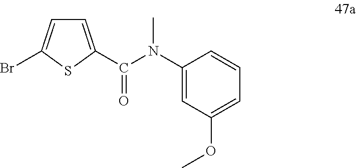 [SMILES: COc1cccc(N(C)C(=O)c2ccc(Br)s2)c1]

The title compound was prepared by reaction of 5-bromothiophene-2-carbonyl chloride (225 mg, 1 mmol) and N-methyl-3-methoxyaniline (137 mg, 1 mmol) in presence of triethylamine (0.16 ml, 1.15 mmol) according to method A. Purification by FC (n-hexane/ethyl acetate 6:1) afforded the desired compound as colorless solid (320 mg, 98%); ¹H NMR (CD₃COCD₃): 3.34 (br s, 3H), 3.82 (s, 3H), 6.52 (d, J=4.1 Hz, 1H), 6.92 (d, J=4.1 Hz, 1H), 6.93 (ddd, J=8.0, 1.9, 0.9 Hz, 1H), 6.99 (t, J=2.2 Hz, 1H), 7.03 (ddd, J=8.0, 2.5, 0.9 Hz, 1H), 7.39 (t, J=8.0 Hz, 1H); ¹³C NMR (CD₃COCD₃): 38.9, 55.9, 114.7, 115.2, 118.3, 121.2, 131.0, 131.5, 133.1, 141.3, 145.8, 161.9.

### N,5-bis(3-Methoxyphenyl)-N-methylthiophene-2-carboxamide (47)

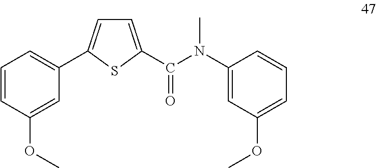 [SMILES: COc1cccc(-c2ccc(C(=O)N(C)c3cccc(OC)c3)s2)c1]

The title compound was prepared by reaction of 5-bromo-N-(3-methoxyphenyl)-N-methylthiophene-2-carboxamide (47a) (75 mg, 0.23 mmol) and 3-methoxyphenylboronic acid (41 mg, 0.27 mmol) with tetrakis(triphenylphosphine) palladium (27 mg, 0.023 mmol) as catalyst according to method B for 5 h. Purification by FC(CH₂Cl₂) afforded the desired compound as colorless solid (80 mg, 98%); ¹H NMR (CD₃COCD₃): 3.38 (s, 3H), 3.82 (s, 3H), 3.83 (s, 3H), 6.58 (d, J=4.1 Hz, 1H), 6.90 (ddd, J=8.2, 2.5, 0.9 Hz, 1H), 6.94 (ddd, J=7.6, 1.6, 0.9 Hz, 1H), 6.98-7.01 (m, 2H), 7.12 (t, J=2.0 Hz, 1H), 7.15 (ddd, J=7.6, 1.6, 0.9 Hz, 1H), 7.15 (d, J=4.1 Hz, 1H), 7.30 (t, J=8.0 Hz, 1H), 7.38 (td, J=8.0, 0.9 Hz, 1H); ¹³C NMR (CD₃COCD₃): 39.0, 55.6, 55.9, 112.0, 114.6, 114.7, 114.8, 118.8, 121.0, 124.1, 131.0, 131.3, 133.1, 135.7, 138.9, 146.6, 148.9, 161.2, 161.8, 162.4.

### 1.48. N,5-bis(3-Hydroxyphenyl)-N-methylthiophene-2-carboxamide (48)

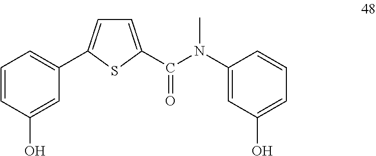 [SMILES: CN(C(=O)c1ccc(-c2cccc(O)c2)s1)c1cccc(O)c1]

The title compound was prepared by reaction of N,5-bis(3-methoxyphenyl)-N-methylthiophene-2-carboxamide (47) (60 mg, 0.17 mmol) with borontrifluoride dimethyl sulfide complex (0.21 ml, 2.04 mmol) according to method C for 8 h. Purification by FC(CH₂Cl₂/CH₃OH 100:1→50:1) yielded the title compound as colorless solid (50 mg, 90%); MS (ESI): 326 (M+H)⁺; ¹H NMR (CD₃COCD₃): 3.36 (s, 3H), 6.63 (d, J=3.8 Hz, 1H), 6.81 (ddd, J=8.2, 2.5, 0.9 Hz, 1H), 6.83-6.86 (m, 2H), 6.91 (ddd, J=8.2, 2.5, 0.9 Hz, 1H), 7.04-7.07 (m, 2H), 7.11 (d, J=4.1 Hz, 1H), 7.21 (t, J=7.9 Hz, 1H), 7.30 (td, J=7.9, 0.6 Hz, 1H), 8.53 (s, 1H), 8.70 (s, 1H); ¹³C NMR (CD₃COCD₃): 39.0, 113.3, 115.9, 116.1, 116.4, 117.9, 119.9, 123.8, 131.1, 131.4, 133.3, 135.7, 138.6, 146.5, 149.2, 158.8, 159.4, 162.4.

### 1.49. 5-(2-Methoxyphenyl)-N-(3-methoxyphenyl)-N-methylthiophene-2-carboxamide (49)

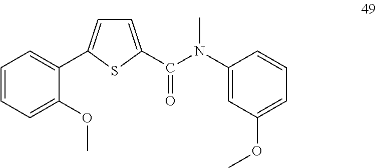 [SMILES: COc1cccc(N(C)C(=O)c2ccc(-c3ccccc3OC)s2)c1]

The title compound was prepared by reaction of 5-bromo-N-(3-methoxyphenyl)-N-methylthiophene-2-carboxamide (47a) (75 mg, 0.23 mmol) and 2-methoxyphenylboronic acid (41 mg, 0.27 mmol) with tetrakis(triphenylphosphine) palladium (27 mg, 0.023 mmol) as catalyst according to method B for 14 h. Purification by FC (n-hexane/ethyl acetate 10:1→6:1) afforded the desired compound as colorless solid (78 mg, 96%); MS (ESI): 354 (M+H)⁺; ¹H NMR (CD₃COCD₃): 3.38 (s, 3H), 3.82 (s, 3H), 3.90, (s, 3H), 6.69 (d, J=4.1 Hz, 1H), 6.93 (ddd, J=8.0, 1.9, 0.9 Hz, 1H), 6.96-7.01 (m, 3H), 7.09 (dd, J=8.0, 0.9 Hz, 1H), 7.28-7.32 (m, 2H), 7.37 (td, J=8.0, 0.6 Hz, 1H), 7.65 (dd, J=8.0, 1.6 Hz, 1H); ¹³C NMR (CD₃COCD₃): 38.9, 54.9, 55.9, 112.9, 114.6, 114.7, 121.2, 121.8, 123.1, 125.2, 128.7, 130.2, 131.3, 132.0, 138.7, 144.6, 146.8, 156.7, 161.8, 162.9.

### 1.50. 5-(2-Hydroxyphenyl)-N-(3-hydroxyphenyl)-N-methylthiophene-2-carboxamide (50)

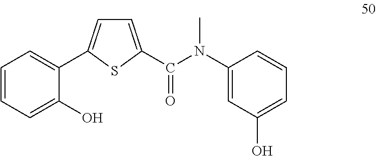 [SMILES: CN(C(=O)c1ccc(-c2ccccc2O)s1)c1cccc(O)c1]

The title compound was prepared by reaction of 5-(2-methoxyphenyl)-N-(3-methoxyphenyl)-N-methylthiophene-2-carboxamide (49) (50 mg, 0.14 mmol) with borontrifluoride dimethyl sulfide complex (0.18 ml, 1.68 mmol) according to method C for 14 h. Purification by FC(CH₂Cl₂/CH₃OH 100:1→50:1) yielded the title compound as colorless solid (40 mg, 88%); ¹H NMR (CD₃COCD₃): 3.36 (s, 3H), 6.59 (d, J=4.3 Hz, 1H), 6.81 (t, J=2.2 Hz, 1H), 6.84 (ddd, J=7.3, 1.8, 0.9 Hz, 1H), 6.86-6.89 (m, 2H), 6.99 (dd, J=8.0, 1.2 Hz, 1H), 7.15 (ddd, J=8.2, 7.3, 1.2 Hz, 1H), 7.28 (t, J=8.0 Hz, 1H), 7.32 (d, J=4.0 Hz, 1H), 7.57 (dd, J=8.0, 1.5 Hz, 1H), 8.67 (s, 1H), 9.18 (s, 1H); ¹³C NMR (CD₃COCD₃): 39.0, 115.9, 117.3, 119.8, 120.9, 121.5, 125.4, 128.9, 130.0, 131.3, 131.9, 138.5, 145.3, 146.8, 154.7, 159.4, 163.0.

### 1.51. N-(3-Methoxyphenyl)-N-methyl-5-phenylthiophene-2-carboxamide (51)

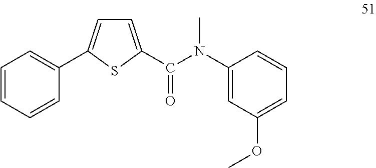 [SMILES: COc1cccc(N(C)C(=O)c2ccc(-c3ccccc3)s2)c1]

The title compound was prepared by reaction of 5-bromo-N-(3-methoxyphenyl)-N-methylthiophene-2-carboxamide (47a) (75 mg, 0.23 mmol) and phenylboronic acid (33 mg, 0.27 mmol) with tetrakis(triphenylphosphine) palladium (27 mg, 0.023 mmol) as catalyst according to method B for 4 h. Purification by FC (n-hexane/ethyl acetate 10:1→6:1) afforded the desired compound as colorless solid (70 mg, 94%); ¹H NMR (CD₃COCD₃): 3.38 (s, 3H), 3.82 (s, 3H), 6.60 (d, J=4.0 Hz, 1H), 6.94 (ddd, J=8.0, 2.1, 0.9 Hz, 1H), 6.99-7.01 (m, 2H), 7.16 (d, J=4.0 Hz, 1H), 7.32 (ddt, J=8.0, 6.3, 1.2 Hz, 1H), 7.36-7.41 (m, 3H), 7.59 (d, J=8.0 Hz, 2H); ¹³C NMR (CD₃COCD₃): 39.0, 55.9, 114.5, 114.7, 121.0, 123.9, 126.6, 129.2, 130.0, 131.3, 133.20, 134.5, 138.9, 146.6, 149.0, 161.8, 162.4.

### 1.52. N-(3-Hydroxyphenyl)-N-methyl-5-phenylthiophene-2-carboxamide (52)

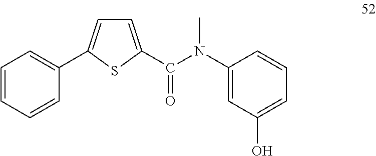 [SMILES: CN(C(=O)c1ccc(-c2ccccc2)s1)c1cccc(O)c1]

The title compound was prepared by reaction of N-(3-methoxyphenyl)-N-methyl-5-phenylthiophene-2-carboxamide (51) (40 mg, 0.12 mmol) with borontrifluoride dimethyl sulfide complex (0.08 ml, 0.72 mmol) according to method C for 6 h. Purification by FC(CH₂Cl₂/CH₃OH 100:1→50:1) yielded the title compound as colorless solid (30 mg, 79%); ¹H NMR (CD₃COCD₃): 3.36 (s, 3H), 6.62 (d, J=4.0 Hz, 1H), 6.83-6.86 (m, 2H), 6.92 (ddd, J=8.2, 2.4, 0.9 Hz, 1H), 7.17 (d, J=4.0 Hz, 1H), 7.28-7.34 (m, 2H), 7.38-7.41 (m, 2H), 7.59 (d, J=7.0 Hz, 2H), 8.74 (s, 1H); ¹³C NMR (CD₃COCD₃): 39.0, 115.9, 116.1, 119.9, 123.9, 126.6, 129.2, 130.0, 131.4, 133.3, 134.5, 138.9, 146.5, 149.1, 159.5, 162.4.

### 1.53. 5-(4-Cyanophenyl)-N-(3-methoxyphenyl)-N-methylthiophene-2-carboxamide (53)

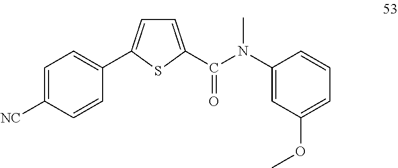 [SMILES: COc1cccc(N(C)C(=O)c2ccc(-c3ccc(C#N)cc3)s2)c1]

The title compound was prepared by reaction of 5-bromo-N-(3-methoxyphenyl)-N-methylthiophene-2-carboxamide (47a) (70 mg, 0.21 mmol) and 4-cyanophenylboronic acid (37 mg, 0.25 mmol) with tetrakis(triphenylphosphine) palladium (24 mg, 0.021 mmol) as catalyst according to method B for 6 h. Purification by FC (n-hexane/ethyl acetate 10:1→6:1) afforded the desired compound as colorless solid (60 mg, 82%); MS (ESI): 349 (M+H)⁺; ¹H NMR (CD₃COCD₃): 3.39 (s, 3H), 3.82 (s, 3H), 6.62 (d, J=4.1 Hz, 1H), 6.95 (ddd, J=7.6, 1.9, 1.2 Hz, 1H), 7.00-7.02 (m, 2H), 7.35 (d, J=4.1 Hz, 1H), 7.37-7.40 (m, 1H), 7.79 (s, 4H); ¹³C NMR (CD₃COCD₃): 39.0, 55.9, 112.3, 114.5, 114.8, 119.1, 121.0, 126.0, 127.1, 131.4, 133.3, 133.8, 138.6, 141.0, 146.4, 146.4, 161.8, 162.1.

### 1.54. 5-(4-Cyanophenyl)-N-(3-hydroxyphenyl)-N-methylthiophene-2-carboxamide (54)

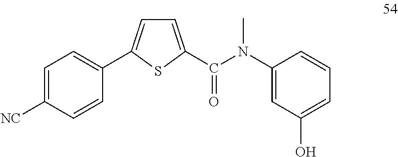 [SMILES: CN(C(=O)c1ccc(-c2ccc(C#N)cc2)s1)c1cccc(O)c1]

The title compound was prepared by reaction of 5-(4-cyanophenyl)-N-(3-methoxyphenyl)-N-methylthiophene-2-carboxamide (53) (40 mg, 0.11 mmol) with borontrifluoride dimethyl sulfide complex (0.07 ml, 0.66 mmol) according to method C for 6 h. Purification by FC(CH₂Cl₂/CH₃OH 100:1→50:1) yielded the title compound as colorless solid (30 mg, 79%); MS (ESI): 335 (M+H)⁺; ¹H NMR (CD₃COCD₃): 3.37 (s, 3H), 6.65 (d, J=4.1 Hz, 1H), 6.84-6.87 (m, 2H), 6.92 (ddd, J=8.0, 2.2, 0.9 Hz, 1H), 7.30 (t, J=8.0 Hz, 1H), 7.36 (d, J=4.1 Hz, 1H), 7.79 (s, 4H), 8.72 (s, 1H); ¹³C NMR (CD₃COCD₃): 39.0, 112.2, 115.9, 116.3, 119.1, 119.9, 126.1, 127.1, 131.5, 133.3, 133.8, 138.6, 141.1, 146.3, 146.4, 159.5, 162.0.

### 1.55. N-(3-Methoxyphenyl)-5-(4-methoxyphenyl)-N-methylthiophene-2-carboxamide (55)

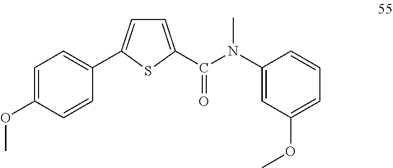 [SMILES: COc1ccc(-c2ccc(C(=O)N(C)c3cccc(OC)c3)s2)cc1]

The title compound was prepared by reaction of 5-bromo-N-(3-methoxyphenyl)-N-methylthiophene-2-carboxamide (47a) (40 mg, 0.12 mmol) and 4-methoxyphenyl-boronic acid (22 mg, 0.14 mmol) with tetrakis(triphenylphosphine) palladium (14 mg, 0.012 mmol) as catalyst according to method B for 6 h. Purification by FC (CH₂Cl₂/CH₃OH 200:1) afforded the desired compound as colorless solid (40 mg, 92%); MS (ESI): 354 (M+H)⁺; ¹H NMR (CD₃COCD₃): 3.37 (s, 3H), 3.81 (s, 3H), 3.82 (s, 3H), 6.55 (d, J=4.1 Hz, 1H), 6.92-6.96 (m, 3H), 6.97-7.01 (m, 2H), 7.02 (d, J=4.1 Hz, 1H), 7.37 (td, J=7.9, 0.6 Hz, 1H), 7.51 (d, J=9.1 Hz, 2H); ¹³C NMR (CD₃COCD₃): 39.0, 55.7, 55.9, 114.5, 115.6, 115.3, 121.0, 122.7, 127.1, 127.9, 131.3, 133.3, 137.8, 146.7, 149.3, 161.0, 161.8, 162.5.

### 1.56. 5-(2-Fluoro-3-methoxyphenyl)-N-(3-methoxyphenyl)-N-methylthiophene-2-carboxamide (56)

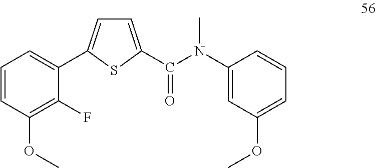 [SMILES: COc1cccc(N(C)C(=O)c2ccc(-c3cccc(OC)c3F)s2)c1]

The title compound was prepared by reaction of 5-bromo-N-(3-methoxyphenyl)-N-methylthiophene-2-carboxamide (47a) (40 mg, 0.12 mmol) and 2-fluoro-3-methoxyphenylboronic acid (25 mg, 0.14 mmol) with tetrakis(triphenylphosphine) palladium (14 mg, 0.012 mmol) as catalyst according to method B for 14 h. Purification by FC (n-hexane/ethyl acetate 10:1→6:1) afforded the desired compound as colorless solid (30 mg, 66%); MS (ESI): 372 (M+H)⁺; ¹H NMR (CD₃COCD₃): 3.38 (s, 3H), 3.82 (s, 3H), 3.90 (s, 3H), 6.65 (dd, J=4.0, 1.0 Hz, 1H), 6.95 (ddd, J=7.6, 1.8, 1.0 Hz, 1H), 6.99-7.01 (m, 2H), 7.09-7.20 (m, 3H), 7.23 (dd, J=4.0, 1.0 Hz, 1H), 7.37 (dd, J=9.1, 7.9 Hz, 1H); ¹³C NMR (CD₃COCD₃): 39.0, 55.9, 56.7, 114.0, 114.1, 114.6, 114.8, 120.4, 121.1, 122.7, 122.8, 125.5, 126.9, 127.0, 131.3, 132.5, 140.0, 141.7, 146.5, 148.8, 149.5, 149.6, 161.8, 162.4.

### 1.57. 5-(2,4-Dimethoxyphenyl)-N-(3-methoxyphenyl)-N-methylthiophene-2-carboxamide (57)

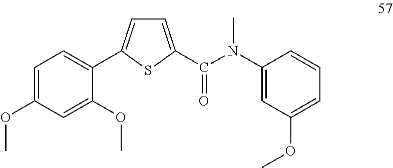 [SMILES: COc1cccc(N(C)C(=O)c2ccc(-c3ccc(OC)cc3OC)s2)c1]

The title compound was prepared by reaction of 5-bromo-N-(3-methoxyphenyl)-N-methylthiophene-2-carboxamide (47a) (40 mg, 0.12 mmol) and 2,4-dimethoxyphenylboronic acid (27 mg, 0.14 mmol) with tetrakis(triphenylphosphine) palladium (14 mg, 0.012 mmol) as catalyst according to method B for 14 h. Purification by FC (n-hexane/ethyl acetate 10:1→6:1) afforded the desired compound as colorless solid (35 mg, 74%); MS (ESI): 384 (M+H)⁺; ¹H NMR (CD₃COCD₃): 3.37 (s, 3H), 3.82 (s, 3H), 3.83 (s, 3H), 3.89 (s, 3H), 6.57 (ddd, J=8.5, 2.4 Hz, 1H), 6.63-6.64 (m, 2H), 6.92 (ddd, J=7.9, 2.1, 0.9 Hz, 1H), 6.97 (t, J=2.1 Hz, 1H), 7.00 (ddd, J=8.2 2.7, 0.9 Hz, 1H), 7.15 (d, J=4.3 Hz, 1H), 7.37 (t, J=8.1 Hz, 1H), 7.55 (d, J=8.5 Hz, 1H); ¹³C NMR (CD₃COCD₃): 38.9, 55.8, 55.9, 99.6, 106.8, 114.5, 114.7, 116.1, 121.2, 123.9, 129.6, 131.2, 132.0, 137.3, 145.1, 146.9, 157.9, 161.7, 162.0, 163.0.

### 1.58. 5-(3-(Dimethylamino)phenyl)-N-(3-methoxyphenyl)-N-methylthiophene-2-carboxamide (58)

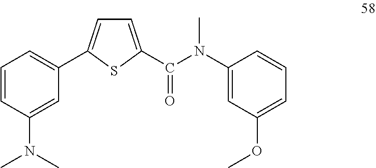 [SMILES: COc1cccc(N(C)C(=O)c2ccc(-c3cccc(N(C)C)c3)s2)c1]

The title compound was prepared by reaction of 5-bromo-N-(3-methoxyphenyl)-N-methylthiophene-2-carboxamide (47a) (49 mg, 0.15 mmol) and 3-(dimethylamino)phenylboronic acid (30 mg, 0.18 mmol) with tetrakis(triphenylphosphine) palladium (17 mg, 0.015 mmol) as catalyst according to method B for 14 h. Purification by FC (n-hexane/ethyl acetate 10:1→6:1) afforded the desired compound as yellowish solid (26 mg, 47%); MS (ESI): 367 (M+H)⁺; ¹H NMR (CD₃COCD₃): 2.96 (s, 6H), 3.38 (s, 3H), 3.82 (s, 3H), 6.56 (d, J=4.0 Hz, 1H), 6.72 (dd, J=8.2, 2.4 Hz, 1H), 6.86 (d, J=7.6 Hz, 1H), 6.89 (t, J=2.0 Hz, 1H), 6.94 (d, J=7.6 Hz, 1H), 6.98-7.01 (m, 2H), 7.10 (d, J=4.0 Hz, 1H), 7.19 (t, J=7.9 Hz, 1H), 7.37 (t, J=7.9 Hz, 1H); ¹³C NMR (CD₃COCD₃): 39.0, 40.5, 55.9, 110.3, 113.5, 114.6, 114.8, 121.0, 123.5, 130.5, 131.3, 133.0, 135.0, 138.3, 146.7, 150.4, 152.0, 161.8, 162.5.

### 1.59. 5-(3-Fluorophenyl)-N-(3-methoxyphenyl)-N-methylthiophene-2-carboxamide (59)

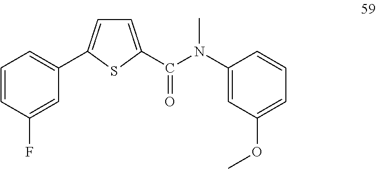 [SMILES: COc1cccc(N(C)C(=O)c2ccc(-c3cccc(F)c3)s2)c1]

The title compound was prepared by reaction of 5-bromo-N-(3-methoxyphenyl)-N-methylthiophene-2-carboxamide (47a) (49 mg, 0.15 mmol) and 3-fluorophenylboronic acid (25 mg, 0.18 mmol) with tetrakis(triphenylphosphine) palladium (17 mg, 0.015 mmol) as catalyst according to method B for 8 h. Purification by FC (n-hexane/ethyl acetate 10:1→6:1) afforded the desired compound as colorless solid (40 mg, 78%); MS (ESI): 342 (M+H)⁺; ¹H NMR (CD₃COCD₃): 3.38 (s, 3H), 3.82 (s, 3H), 6.59 (d, J=4.1 Hz, 1H), 6.94-6.96 (m, 1H), 6.99-7.02 (m, 2H), 7.08-7.12 (m, 1H), 7.23 (d, J=3.8 Hz, 1H), 7.34-7.46 (m, 4H); ¹³C NMR (CD₃COCD₃): 39.0, 55.9, 113.0, 113.2, 114.5, 114.8, 115.7, 115.9, 121.0, 122.6, 124.9, 131.4, 131.9, 132.0, 133.1, 136.7, 136.8, 139.7, 146.5, 147.3, 161.8, 162.2, 163.0, 165.0.

### 1.60. 5-(3,4-Difluorophenyl)-N-(3-methoxyphenyl)-N-methylthiophene-2-carboxamide (60)

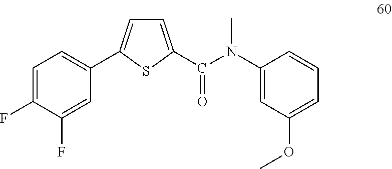 [SMILES: COc1cccc(N(C)C(=O)c2ccc(-c3ccc(F)c(F)c3)s2)c1]

The title compound was prepared by reaction of 5-bromo-N-(3-methoxyphenyl)-N-methylthiophene-2-carboxamide (47a) (49 mg, 0.15 mmol) and 3,4-difluorophenylboronic acid (30 mg, 0.19 mmol) with tetrakis(triphenylphosphine) palladium (17 mg, 0.015 mmol) as catalyst according to method B for 14 h. Purification by FC (n-hexane/ethyl acetate 10:1→6:1) afforded the desired compound as colorless solid (35 mg, 65%); MS (ESI): 360 (M+H)⁺; ¹H NMR (CD₃COCD₃): 3.38 (s, 3H), 3.82 (s, 3H), 6.57 (d, J=4.1 Hz, 1H), 6.95 (ddd, J=7.6, 1.8, 0.9 Hz, 1H), 6.99-7.02 (m, 2H), 7.18 (d, J=4.1 Hz, 1H), 7.33-7.43 (m, 3H), 7.56 (ddd, J=12.0, 7.6, 2.2 Hz, 1H); ¹³C NMR (CD₃COCD₃): 39.0, 55.9, 114.5, 114.8, 115.5, 118.9, 119.1, 121.0, 123.4, 123.4, 123.5, 131.4, 132.1, 133.1, 139.9, 146.4, 146.5, 149.9, 150.0, 150.2, 150.3, 151.8, 151.9, 152.2, 152.3, 154.4, 161.8, 162.2.

### 1.61. 5-(3-Fluoro-4-methoxyphenyl)-N-(3-methoxyphenyl)-N-methylthiophene-2-carboxamide (61)

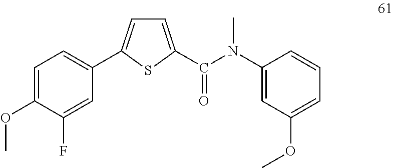 [SMILES: COc1cccc(N(C)C(=O)c2ccc(-c3ccc(OC)c(F)c3)s2)c1]

The title compound was prepared by reaction of 5-bromo-N-(3-methoxyphenyl)-N-methylthiophene-2-carboxamide (47a) (40 mg, 0.12 mmol) and 3-fluoro-4-methoxyphenylboronic acid (25 mg, 0.14 mmol) with tetrakis(triphenylphosphine) palladium (14 mg, 0.015 mmol) as catalyst according to method B for 6 h. Purification by FC (n-hexane/ethyl acetate 10:1→6:1) afforded the desired compound as colorless solid (40 mg, 72%); MS (ESI): 372 (M+H)⁺; ¹H NMR (CD₃COCD₃): 3.38 (s, 3H), 3.82 (s, 3H), 3.91 (s, 3H), 6.55 (d, J=4.0 Hz, 1H), 6.94 (ddd, J=7.9, 1.9, 0.9 Hz, 1H), 6.99-7.01 (m, 2H), 7.08 (d, J=4.0 Hz, 1H), 7.15 (t, J=8.5 Hz, 1H), 7.33-7.39 (m, 3H); ¹³C NMR (CD₃COCD₃): 39.0, 55.9, 56.6, 114.0, 114.2, 114.6, 114.7, 115.0, 115.1, 121.0, 122.9, 123.0, 123.6, 127.6, 127.7, 131.3, 133.2, 138.6, 146.6, 147.8, 148.8, 152.2, 154.2, 161.8, 162.3.

### 1.62. 5-(4-Fluoro-3-methoxyphenyl)-N-(3-methoxyphenyl)-N-methylthiophene-2-carboxamide (62)

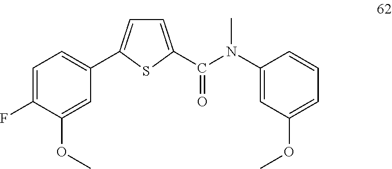 [SMILES: COc1cccc(N(C)C(=O)c2ccc(-c3ccc(F)c(OC)c3)s2)c1]

The title compound was prepared by reaction of 5-bromo-N-(3-methoxyphenyl)-N-methylthiophene-2-carboxamide (47a) (40 mg, 0.12 mmol) and 4-fluoro-3-methoxyphenylboronic acid (25 mg, 0.14 mmol) with tetrakis(triphenylphosphine) palladium (14 mg, 0.015 mmol) as catalyst according to method B for 8 h. Purification by FC (n-hexane/ethyl acetate 10:1→6:1) afforded the desired compound as colorless solid (43 mg, 96%); MS (ESI): 372 (M+H)⁺; ¹H NMR (CD₃COCD₃): 3.38 (s, 3H), 3.82 (s, 3H), 3.95 (s, 3H), 6.52 (d, J=4.0 Hz, 1H), 6.94 (ddd, J=7.2, 2.2, 0.9 Hz, 1H), 6.98-7.01 (m, 2H), 7.11-7.17 (m, 3H), 7.32 (dd, J=8.0, 2.1 Hz, 1H), 7.38 (td, J=8.0, 0.9 Hz, 1H); ¹³C NMR (CD₃COCD₃): 39.0, 55.9, 56.6, 112.2, 114.6, 117.1, 117.3, 119.2, 121.0, 124.2, 131.3, 131.4, 133.0, 139.1, 146.6, 148.1, 149.1, 152.2, 154.2, 161.8, 162.4.

### 1.63. N-(3-methoxyphenyl)-N-methyl-5-(3-(methylthio)phenyl)thiophene-2-carboxamide (63)

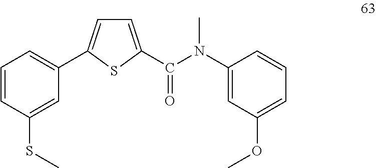 [SMILES: COc1cccc(N(C)C(=O)c2ccc(-c3cccc(SC)c3)s2)c1]

The title compound was prepared by reaction of 5-bromo-N-(3-methoxyphenyl)-N-methylthiophene-2-carboxamide (47a) (33 mg, 0.1 mmol) and 3-(thiomethyl)phenylboronic acid (23 mg, 0.14 mmol) with tetrakis(triphenylphosphine) palladium (12 mg, 0.01 mmol) as catalyst according to method B for 8 h. Purification by FC (n-hexane/ethyl acetate 10:17:1) afforded the desired compound as yellowish solid (22 mg, 59%); ¹H NMR (CD₃COCD₃): 2.52 (s, 3H), 3.38 (s, 3H), 3.82 (s, 3H), 6.59 (d, J=4.0 Hz, 1H), 6.94 (ddd, J=8.0, 1.9, 0.9 Hz, 1H), 6.98-7.02 (m, 2H), 7.18 (d, J=4.0 Hz, 1H), 7.23 (dt, J=6.9, 1.9 Hz, 1H), 7.30-7.35 (m, 2H), 7.38 (td, J=8.0, 0.9 Hz, 1H), 7.44-7.45 (m, 1H); ¹³C NMR (CD₃COCD₃): 15.3, 29.0, 55.9, 114.6, 114.7, 121.0, 123.2, 123.9, 124.3, 126.8, 130.4, 131.4, 133.1, 135.1, 139.2, 140.9, 146.6, 148.4, 161.8, 162.3.

### 1.64. N-(3-methoxyphenyl)-N-methyl-5-m-tolylthiophene-2-carboxamide (64)

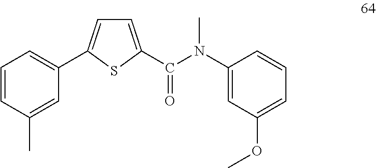 [SMILES: COc1cccc(N(C)C(=O)c2ccc(-c3cccc(C)c3)s2)c1]

The title compound was prepared by reaction of 5-bromo-N-(3-methoxyphenyl)-N-methylthiophene-2-carboxamide (47a) (33 mg, 0.1 mmol) and 3-methylphenylboronic acid (19 mg, 0.14 mmol) with tetrakis(triphenylphosphine) palladium (12 mg, 0.01 mmol) as catalyst according to method B for 8 h. Purification by FC (n-hexane/ethyl acetate 10:1→6:1) afforded the desired compound as colorless solid (26 mg, 77%); MS (ESI): 338 (M+H)⁺; ¹H NMR (CD₃COCD₃): 2.34 (s, 3H), 3.38 (s, 3H), 3.81 (s, 3H), 6.57 (d, J=4.0 Hz, 1H), 6.94 (ddd, J=7.9, 2.2, 0.9 Hz, 1H), 6.98-7.01 (m, 2H), 7.13 (d, J=4.0 Hz, 1H), 7.14-7.16 (m, 1H), 7.27 (t, J=7.9 Hz, 1H), 7.36-7.39 (m, 2H), 7.41-7.42 (m, 1H); ¹³C NMR (CD₃COCD₃): 21.3, 39.0, 55.9, 114.5, 114.6, 121.0, 123.7, 123.8, 127.2, 129.9, 130.0, 131.3, 133.1, 134.4, 138.8, 139.6, 146.7, 149.2, 161.8, 162.5.

### 1.65. 5-(2,6-Difluoro-3-methoxyphenyl)-N-(3-methoxyphenyl)-N-methylthiophene-2-carboxamide (65)

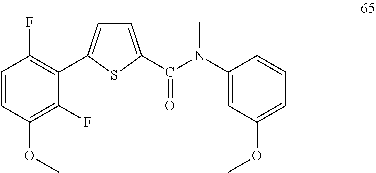 [SMILES: COc1cccc(N(C)C(=O)c2ccc(-c3c(F)ccc(OC)c3F)s2)c1]

The title compound was prepared by reaction of 5-bromo-N-(3-methoxyphenyl)-N-methylthiophene-2-carboxamide (47a) (33 mg, 0.1 mmol) and 2,6-difluoro-3-methoxyphenyl boronic acid (26 mg, 0.14 mmol) with tetrakis(triphenylphosphine) palladium (12 mg, 0.01 mmol) as catalyst according to method B for 14 h. Purification by FC (n-hexane/ethyl acetate 10:1) afforded the desired compound as colorless solid (10 mg, 26%); MS (ESI): 390 (M+H)⁺; ¹H NMR (CD₃COCD₃): 3.40 (s, 3H), 3.82 (s, 3H), 3.90 (s, 3H), 6.71 (dt, J=4.1, 0.9 Hz, 1H), 6.95 (ddd, J=7.6, 1.9, 0.9 Hz, 1H), 6.99-7.01 (m, 2H), 7.05 (ddd, J=11.4, 9.1, 2.2 Hz, 1H), 7.16 (td, J=9.1, 5.0 Hz, 1H), 7.23 (dt, J=4.1, 1.1 Hz, 1H), 7.36-7.39 (m, 1H); ¹³C NMR (CD₃COCD₃): 39.0, 55.9, 57.1, 111.5, 111.7, 114.0, 114.1, 114.6, 114.9, 121.1, 129.9 131.4, 131.9, 134.4, 141.0, 145.9, 146.0, 146.4, 148.8, 149.1, 149.2, 149.9, 150.8, 152.8, 154.7, 161.8, 162.3.

### 1.66. 5-(2-Fluoro-3-methylphenyl)-N-(3-methoxyphenyl)-N-methylthiophene-2-carboxamide (66)

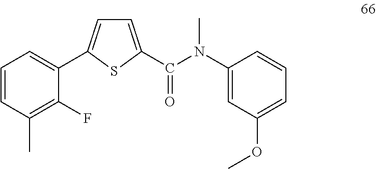 [SMILES: COc1cccc(N(C)C(=O)c2ccc(-c3cccc(C)c3F)s2)c1]

The title compound was prepared by reaction of 5-bromo-N-(3-methoxyphenyl)-N-methylthiophene-2-carboxamide (47a) (49 mg, 0.15 mmol) and 2-fluoro-3-methylphenylboronic acid (28 mg, 0.18 mmol) with tetrakis(triphenylphosphine) palladium (17 mg, 0.015 mmol) as catalyst according to method B for 14 h. Purification by FC (n-hexane/ethyl acetate 10:1) afforded the desired compound as colorless solid (50 mg, 94%); ¹H NMR (CD₃COCD₃): 2.29 (d, J=2.5 Hz, 3H), 3.39 (s, 3H), 3.81 (s, 3H), 6.63 (dd, J=4.1, 0.9 Hz, 1H), 6.94 (ddd, J=7.9, 1.9, 0.9 Hz, 1H), 6.98-7.01 (m, 2H), 7.11 (t, J=7.9 Hz, 1H), 7.22 (dd, J=4.1, 0.9 Hz, 1H), 7.23-7.25 (m, 1H), 7.36-7.39 (m, 1H), 7.45-7.48 (m, 1H). ¹³C NMR (CD₃COCD₃): 14.6, 39.0, 55.9, 114.5, 114.7, 121.0, 121.8, 121.9, 123.9, 125.3, 126.6, 126.7, 126.7, 126.8, 127.1, 127.2, 131.3, 132.2, 132.3, 132.5, 139.7, 139.8, 142.1, 146.6, 157.4, 159.3, 161.8, 162.4.

### 1.67. 5-(3-Methoxyphenyl)-N-methyl-N-phenylthiophene-2-carboxamide (67)

### 5-Bromo-N-methyl-N-phenylthiophene-2-carboxamide (67a)

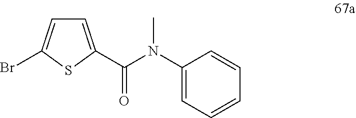 [SMILES: CN(C(=O)c1ccc(Br)s1)c1ccccc1]

The title compound was prepared by reaction of 5-bromothiophene-2-carbonyl chloride (113 mg, 0.5 mmol) and N-methylaniline (54 mg, 0.5 mmol) in presence of triethylamine (0.08 ml, 0.58 mmol) according to method A. Purification by FC (CH₂Cl₂) yielded the title compound as colorless solid (146 mg, 99%). MS (ESI): 296 (M+H)⁺; ¹H NMR (CD₃COCD₃): 3.38 (s, 3H), 6.43 (d, J=4.1 Hz, 1H), 6.90 (d, J=4.1 Hz, 1H), 7.27-7.40 (m, 2H), 7.44-7.52 (m, 3H); ¹³C NMR (CD₃COCD₃): 39.0, 118.3, 129.2, 129.4, 130.9, 131.0, 133.1, 141.4, 144.8, 161.3.

### 5-(3-Methoxyphenyl)-N-methyl-N-phenylthiophene-2-carboxamide (67)

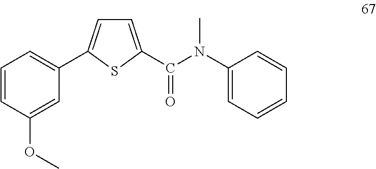 [SMILES: COc1cccc(-c2ccc(C(=O)N(C)c3ccccc3)s2)c1]

The title compound was prepared by reaction of 5-bromo-N-methyl-N-phenylthiophene-2-carboxamide (67a) (74 mg, 0.25 mmol) and 3-methoxyphenylboronic acid (45 mg, 0.3 mmol) with tetrakis(triphenylphosphine) palladium (29 mg, 0.025 mmol) as catalyst according to method B for 4 h. Purification by FC (n-hexane/ethyl acetate 10:1→6:1) afforded the desired compound as colorless solid (65 mg, 76%); MS (ESI): 324 (M+H)⁺; ¹H NMR (CD₃COCD₃): 3.39 (s, 3H), 3.83 (s, 3H), 6.50 (d, J=4.1 Hz, 1H), 6.90 (ddd, J=8.2, 2.5, 0.9 Hz, 1H), 7.10 (t, J=2.2 Hz, 1H), 7.12-7.14 (m, 2H), 7.30 (t, J=8.2 Hz, 1H), 7.38-7.40 (m, 2H), 7.42-7.45 (m, 1H), 7.47-7.51 (m, 2H); ¹³C NMR (CD₃COCD₃): 39.0, 55.6, 112.0, 114.8, 119.0, 124.0, 128.9, 129.0, 130.7, 131.0, 133.1, 135.7, 138.9, 145.6, 148.9, 161.2, 162.4.

### 1.68. 5-(3-Hydroxyphenyl)-N-methyl-N-phenylthiophene-2-carboxamide (68)

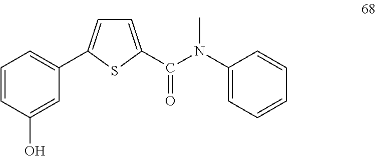 [SMILES: CN(C(=O)c1ccc(-c2cccc(O)c2)s1)c1ccccc1]

The title compound was prepared by reaction of 5-(3-methoxyphenyl)-N-methyl-N-phenylthiophene-2-carboxamide (67) (45 mg, 0.14 mmol) with borontrifluoride dimethyl sulfide complex (0.09 ml, 0.84 mmol) according to method C for 6 h. Purification by FC(CH₂Cl₂/CH₃OH 100:1→50:1) yielded the title compound as colorless solid (38 mg, 88%); MS (ESI): 310 (M+H)⁺; ¹H NMR (CD₃COCD₃): 3.39 (s, 3H), 6.52 (d, J=4.1 Hz, 1H), 6.81 (ddd, J=8.0, 2.5, 0.9 Hz, 1H), 7.02-7.06 (m, 2H), 7.08 (d, J=4.1 Hz, 1H), 7.21 (t, J=8.0 Hz, 1H), 7.37-7.40 (m, 2H), 7.41-7.45 (m, 1H), 7.47-7.51 (m, 2H), 8.50 (s, 1H). ¹³C NMR (CD₃COCD₃): 39.0, 113.3, 116.4, 117.9, 123.7, 128.9, 129.0, 130.7, 131.1, 133.2, 135.7, 138.6, 145.5, 149.2, 158.8, 162.5.

### 1.69. N-Methyl-N,5-diphenylthiophene-2-carboxamide (69)

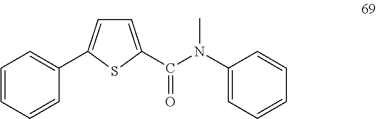 [SMILES: CN(C(=O)c1ccc(-c2ccccc2)s1)c1ccccc1]

The title compound was prepared by reaction of 5-bromo-N-methyl-N-phenylthiophene-2-carboxamide (67a) (57 mg, 0.19 mmol) and phenylboronic acid (35 mg, 0.23 mmol) with tetrakis(triphenylphosphine) palladium (22 mg, 0.019 mmol) as catalyst according to method B for 4 h. Purification by FC (n-hexane/ethyl acetate 10:1→6:1) afforded the desired compound as colourless solid (50 mg, 90%); MS (ESI): 294 (M+H)⁺; ¹H NMR (CD₃COCD₃): 3.39 (s, 3H), 6.52 (d, J=4.1 Hz, 1H), 7.14 (d, J=4.1 Hz, 1H), 7.30-7.34 (m, 1H), 7.37-7.41 (m, 4H), 7.42-7.45 (m, 1H), 7.47-7.51 (m, 2H), 7.56-7.58 (m, 2H); ¹³C NMR (CD₃COCD₃): 39.0, 123.8, 126.6, 128.9, 129.0, 129.2, 130.0, 130.7, 133.3, 134.4, 138.9, 145.6, 149.1, 162.5.

### 1.70. 5-(3-Methoxyphenyl)-N-methyl-N-m-tolylthiophene-2-carboxamide (70)

### 5-Bromo-N-methyl-N-m-tolylthiophene-2-carboxamide (70a)

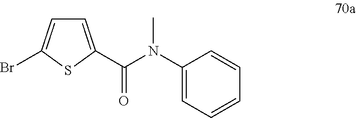 [SMILES: CN(C(=O)c1ccc(Br)s1)c1ccccc1]

The title compound was prepared by reaction of 5-bromothiophene-2-carbonyl chloride (225 mg, 1 mmol) and N-methyl-m-toluidine (121 mg, 1 mmol) in presence of triethylamine (0.16 ml, 1.15 mmol) according to method A. Purification by FC(CH₂Cl₂) yielded the desired compound as colourless solid (300 mg, 97%); MS (ESI): 309 (M+H)⁺; ¹H NMR (CD₃COCD₃): 2.37 (s, 3H), 3.34 (s, 3H), 6.46 (d, J=4.1 Hz, 1H), 6.90 (d, J=4.1 Hz, 1H), 7.14-7.16 (m, 1H), 7.22 (s, 1H), 7.28-7.29 (m, 1H), 7.37 (t, J=7.7 Hz, 1H); ¹³C NMR (CD₃COCD₃): 21.2, 39.0, 118.3, 126.1, 129.6, 130.1, 130.6, 131.0, 133.1, 141.0, 141.4, 144.7, 161.2.

### 5-(3-Methoxyphenyl)-N-methyl-N-m-tolylthiophene-2-carboxamide (70)

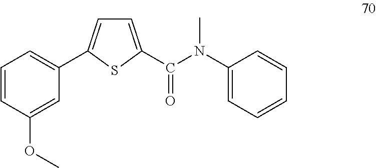 [SMILES: COc1cccc(-c2ccc(C(=O)N(C)c3ccccc3)s2)c1]

The title compound was prepared by reaction of 5-bromo-N-methyl-N-m-tolylthiophene-2-carboxamide (70a) (78 mg, 0.25 mmol) and 3-methoxyphenylboronic acid (45 mg, 0.3 mmol) with tetrakis(triphenylphosphine) palladium (29 mg, 0.025 mmol) as catalyst according to method B for 4 h. Purification by FC (n-hexane/ethyl acetate 25:1→10:1) afforded the desired compound as colorless solid (65 mg, 77%); MS (ESI): 338 (M+H)⁺; ¹H NMR (CD₃COCD₃): 2.36 (s, 3H), 3.37 (s, 3H), 3.83 (s, 3H), 6.51 (d, J=4.1 Hz, 1H), 6.90 (ddd, 3=8.2, 2.5, 0.9 Hz, 1H), 7.10-7.11 (m, 1H), 7.13-7.17 (m, 3H), 7.23 (s, 1H), 7.26 (d, J=7.6 Hz, 1H), 7.30 (t, J=7.6 Hz, 1H), 7.36 (t, J=7.7 Hz, 1H); ¹³C NMR (CD₃COCD₃): 21.2, 39.1, 55.6, 112.0, 114.8, 119.0, 124.0, 126.0, 129.5, 129.6, 130.5, 131.0, 133.1, 135.7, 139.1, 140.7, 145.4, 148.9, 161.2, 162.4.

### 1.71. 5-(3-Hydroxyphenyl)-N-methyl-N-m-tolylthiophene-2-carboxamide (71)

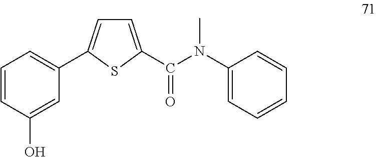 [SMILES: CN(C(=O)c1ccc(-c2cccc(O)c2)s1)c1ccccc1]

The title compound was prepared by reaction of 5-(3-methoxyphenyl)-N-methyl-N-m-tolylthiophene-2-carboxamide (70) (40 mg, 0.12 mmol) with borontrifluoride dimethyl sulfide complex (0.08 ml, 0.72 mmol) according to method C for 14 h. Purification by FC(CH₂Cl₂/CH₃OH 100:1→50:1) yielded the title compound as colorless solid (30 mg, 79%); MS (ESI): 324 (M+H)⁺; ¹H NMR (CD₃COCD₃): 2.36 (s, 3H), 3.37 (s, 3H), 6.52 (d, J=4.1 Hz, 1H), 6.81 (ddd, J=8.0, 2.5, 0.9 Hz, 1H), 7.03-7.06 (m, 2H), 7.08 (d, J=4.1 Hz, 1H), 7.16 (d, J=8.0 Hz, 1H), 7.20 (d, J=7.6 Hz, 1H), 7.22 (s, 1H), 7.26 (d, J=7.6 Hz, 1H), 7.36 (t, J=8.0 Hz, 1H), 8.51 (s, 1H); ¹³C NMR (CD₃COCD₃): 21.2, 39.1, 113.4, 116.4, 117.9, 123.7, 126.0, 129.5, 129.6, 130.5, 131.1, 133.2, 135.7, 138.8, 140.7, 145.4, 149.2, 158.8, 162.4.

### 1.72. 5-(2-Fluoro-3-methoxyphenyl)-N-methyl-N-(m-tolylthiophene)-2-carboxamide (72)

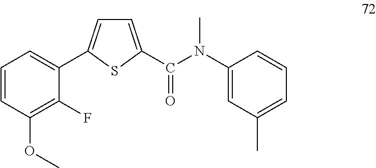 [SMILES: COc1cccc(-c2ccc(C(=O)N(C)c3cccc(C)c3)s2)c1F]

The title compound was prepared by reaction of 5-bromo-N-methyl-N-m-tolylthiophene-2-carboxamide (70a) (46 mg, 0.15 mmol) and 2-fluoro-3-methoxyphenyl-boronic acid (31 mg, 0.18 mmol) with tetrakis(triphenylphosphine) palladium (17 mg, 0.015 mmol) as catalyst according to method B for 14 h. Purification by FC (n-hexane/ethyl acetate 10:1) afforded the desired compound as colourless solid (45 mg, 85%); MS (ESI): 356 (M+H)⁺; ¹H NMR (CD₃COCD₃): 2.36 (s, 3H), 3.38 (s, 3H), 3.90 (s, 3H), 6.58 (dd, J=4.0, 1.0 Hz, 1H), 7.08-7.19 (m, 4H), 7.21 (dd, J=4.1, 1.0 Hz, 1H), 7.23-7.27 (m, 2H), 7.36 (t, J=7.7 Hz, 1H); ¹³C NMR (CD₃COCD₃): 21.2, 39.1, 56.7, 114.0, 118.8, 120.4, 122.7, 122.8, 125.4, 126.0, 126.9, 129.5, 129.7, 130.5, 132.6, 140.1, 140.8, 141.7, 145.3, 148.8, 149.5, 150.8, 162.3.

### 1.73. 5-(2-Fluoro-3-methoxyphenyl)-N-(4-methoxyphenyl)-N-methylthiophene-2-carboxamide (73)

### 5-Bromo-N-(4-methoxyphenyl)-N-methylthiophene-2-carboxamide (73a)

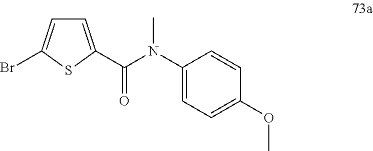 [SMILES: COc1ccc(N(C)C(=O)c2ccc(Br)s2)cc1]

The title compound was prepared by reaction of 5-bromothiophene-2-carbonyl chloride (113 mg, 0.5 mmol) and N-methyl-4-methoxyaniline (69 mg, 0.5 mmol) in presence of triethylamine (0.08 ml, 0.58 mmol) according to method A. Purification by FC (n-hexane/ethyl acetate 6:1) yielded the product as colorless solid (160 mg, 98%). MS (ESI): 326 (M+H)⁺; ¹H NMR (CD₃COCD₃): 3.31 (s, 3H), 3.86 (s, 3H), 6.55 (d, J=4.1 Hz, 1H), 6.93 (d, J=4.1 Hz, 1H), 7.04 (d, J=9.1 Hz, 2H), 7.30 (d, J=9.1 Hz, 2H); ¹³C NMR (CD₃COCD₃): 39.1, 55.9, 116.0, 118.4, 130.5, 130.9, 133.3, 137.3, 141.1, 160.8, 161.3.

### 5-(2-Fluoro-3-methoxyphenyl)-N-(4-methoxyphenyl)-N-methylthiophene-2-carboxamide (73)

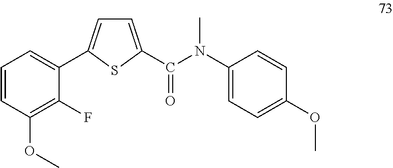 [SMILES: COc1ccc(N(C)C(=O)c2ccc(-c3cccc(OC)c3F)s2)cc1]

The title compound was prepared by reaction of 5-bromo-N-(4-methoxyphenyl)-N-methylthiophene-2-carboxamide (73a) (49 mg, 0.15 mmol) and 2-fluoro-3-methoxyphenylboronic acid (31 mg, 0.18 mmol) with tetrakis(triphenylphosphine) palladium (17 mg, 0.015 mmol) as catalyst according to method B for 14 h. Purification by FC (n-hexane/ethyl acetate 10:1→5:1) afforded the desired compound as colorless solid (40 mg, 72%); MS (ESI): 372 (M+H)⁺; ¹H NMR (CD₃COCD₃): 3.35 (s, 3H), 3.85 (s, 3H), 3.90 (s, 3H), 6.62 (dd, J=4.1, 1.3 Hz, 1H), 7.03 (d, J=8.8 Hz, 2H), 7.08-7.18 (m, 3H), 7.23 (dd, J=4.1, 0.9 Hz, 1H), 7.31 (d, J=8.8 Hz, 2H); ¹³C NMR (CD₃COCD₃): 39.2, 55.9, 56.7, 114.0, 115.8, 120.4, 120.5, 122.7, 122.8, 125.4, 125.5, 126.9, 127.0, 130.2, 132.6, 138.0, 140.1, 141.7, 141.8, 148.8, 149.5, 149.6, 150.8, 160.5, 162.4.

### 1.74. N-(2-fluorophenyl)-N-methyl-5-m-tolylthiophene-2-carboxamide (74)

### 5-Bromo-N-(2-fluorophenyl)-N-methylthiophene-2-carboxamide (74a)

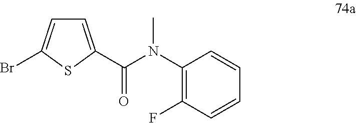 [SMILES: CN(C(=O)c1ccc(Br)s1)c1ccccc1F]

The title compound was prepared by reaction of 5-bromothiophene-2-carbonyl chloride (225 mg, 1 mmol) and 2-fluoro-N-methylaniline (140 mg, 1 mmol) in presence of triethylamine (0.16 ml, 1.16 mmol) according to method A. Purification by FC (n-hexane/ethyl acetate 6:1) yielded the product as colorless oil (300 mg, 94%). MS (ESI): 314 (M+H)⁺; ¹H NMR (CD₃COCD₃): 3.34 (s, 3H), 6.61 (d, J=4.1 Hz, 1H), 6.96 (d, J=4.1 Hz, 1H), 7.29-7.37 (m, 2H), 7.51-7.57 (m, 2H); ¹³C NMR (CD₃COCD₃): 38.2, 117.8, 118.0, 118.4, 126.6, 131.2, 131.5, 131.7, 131.8, 132.1, 132.2, 132.8, 140.4, 158.3, 160.3, 161.8.

### N-(2-fluorophenyl)-N-methyl-5-m-tolylthiophene-2-carboxamide (74)

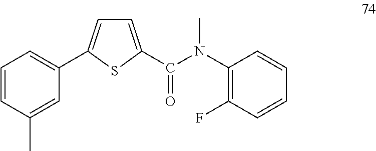 [SMILES: Cc1cccc(-c2ccc(C(=O)N(C)c3ccccc3F)s2)c1]

The title compound was prepared by reaction of 5-bromo-N-(2-fluorophenyl)-N-methylthiophene-2-carboxamide (74a) (47 mg, 0.15 mmol) and m-tolylboronic acid (25 mg, 0.18 mmol) with tetrakis(triphenylphosphine) palladium (17 mg, 0.015 mmol) as catalyst according to method B for 14 h. Purification by FC (n-hexane/ethyl acetate 10:1→5:1) afforded the desired compound as colorless solid (42 mg, 86%); ¹H NMR (CD₃COCD₃): 2.34 (s, 3H), 3.27 (s, 3H), 6.69 (d, J=4.1 Hz, 1H), 7.14-7.16 (m, 2H), 7.27 (t, J=7.9 Hz, 1H), 7.28-7.37 (m, 3H), 7.41 (s, 1H), 7.48-7.53 (m, 1H), 7.55 (td, J=7.9, 1.9 Hz, 1H); ¹³C NMR (CD₃COCD₃): 21.3, 38.2, 116.8, 117.7, 117.9, 123.8, 126.4, 126.5, 127.3, 129.9, 130.1, 131.2, 131.3, 131.4, 132.8, 132.9, 133.0, 134.2, 137.8, 139.7, 149.6, 158.3, 160.3, 162.9.

### 1.75. 543-Methoxyphenyl)-N-methyl-N-(3-(trifluoromethyl)phenyl)thiophene-2-carboxamide (75)

### 5-Bromo-N-methyl-N-(3-(trifluoromethyl)phenyl)thiophene-2-carboxamide (75a)

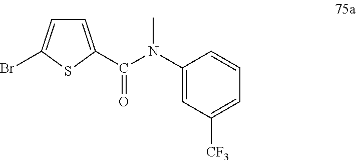 [SMILES: CN(C(=O)c1ccc(Br)s1)c1cccc(C(F)(F)F)c1]

The title compound was prepared by reaction of 5-bromothiophene-2-carbonyl chloride (225 mg, 1 mmol) and N-methyl-3-(trifluoromethyl)aniline (175 mg, 1 mmol) in presence of triethylamine (0.16 ml, 1.15 mmol) according to method A. Purification by FC (n-hexane/ethyl acetate 6:1) yielded the product as colorless solid (360 mg, 99%). MS (ESI): 366 (M+H)⁺; ¹H NMR (CD₃COCD₃): 3.43 (s, 3H), 6.52 (d, J=4.1 Hz, 1H), 6.95 (d, J=4.1 Hz, 1H), 7.70-7.75 (m, 2H), 7.78-7.82 (m, 2H); ¹³C NMR (CD₃COCD₃): 39.0, 118.4, 123.6, 125.7, 125.8, 125.9, 126.0, 131.2, 131.9, 132.4, 132.6, 133.1, 133.2, 140.9, 145.7, 161.5.

### 5-(3-Methoxyphenyl)-N-methyl-N-(3-(trifluoromethyl)phenyl)thiophene-2-carboxamide (75)

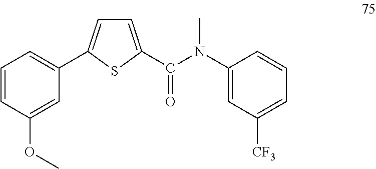 [SMILES: COc1cccc(-c2ccc(C(=O)N(C)c3cccc(C(F)(F)F)c3)s2)c1]

The title compound was prepared by reaction of 5-bromo-N-methyl-N-(3-(trifluoromethyl)phenyl)thiophene-2-carboxamide (75a) (36 mg, 0.1 mmol) and 3-methoxyphenylboronic acid (20 mg, 0.13 mmol) with tetrakis(triphenylphosphine) palladium (12 mg, 0.01 mmol) as catalyst according to method B for 6 h. Purification by FC (n-hexane/ethyl acetate 10:1→5:1) afforded the desired compound as colorless solid (35 mg, 90%); MS (ESI): 392 (M+H)⁺; ¹H NMR (CD₃COCD₃): 3.47 (s, 3H), 3.83 (s, 3H), 6.57 (d, J=4.1 Hz, 1H), 6.91 (ddd, J=8.2, 2.5, 0.9 Hz, 1H), 7.11 (t, J=2.2 Hz, 1H), 7.14 (ddd, J=7.6, 1.6, 0.9 Hz, 1H), 7.19 (d, J=4.1 Hz, 1H), 7.31 (t, J=8.2 Hz, 1H), 7.69-7.78 (m, 3H), 7.81-7.82 (m, 1H); ¹³C NMR (CD₃COCD₃): 39.0, 55.6, 112.0, 115.0, 119.0, 124.1, 125.3, 125.4, 125.8, 125.9, 131.1, 131.7, 132.2, 132.5, 133.0, 133.5, 135.5, 138.2, 146.3, 149.2, 161.2, 162.6.

### 1.76. N-(Biphenyl-3-yl)-5-(3-methoxyphenyl)-N-methylthiophene-2-carboxamide (76)

### N-methylbiphenyl-3-amine (76b)

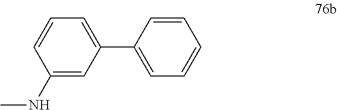

3-Bromo-N-methylaniline (93 mg, 0.5 mmol), phenyl boronic acid (78 mg, 0.6 mmol), sodium carbonate (2 ml, 2N aqueous) and tetrakis(triphenylphosphine) palladium (58 mg, 0.05 mmol) in a DME (2 ml) solution was stirred at 80° C. for 6 hours under nitrogen. The reaction mixture was cooled to rt. The aqueous layer was extracted with dichloromethane. The combined organic layers were washed with brine, dried over sodium sulphate, filtered and concentrated to dryness. The product was purified by FC with hexane/ethyl acetate (15:1) as eluent, yielded as colorless oil (60 mg, 66%); MS (ESI): 184 (M+H)⁺; ¹H NMR (CD₃COCD₃): 2.84 (s, 3H), 5.02 (br s, 1H), 6.60 (ddd, J=8.1, 2.1, 0.9 Hz, 1H), 6.85-6.87 (m, 2H), 7.19 (t, J=8.1 Hz, 1H), 7.30-7.33 (m, 1H), 7.42 (t, J=7.6 Hz, 2H), 7.59-7.61 (m, 2H); ¹³C NMR (CD₃COCD₃): 30.5, 111.2, 112.0, 115.8, 127.7, 127.8, 129.5, 130.2, 142.7, 142.9, 151.5.

### N-(biphenyl-3-yl)-5-bromo-N-methylthiophene-2-carboxamide (76a)

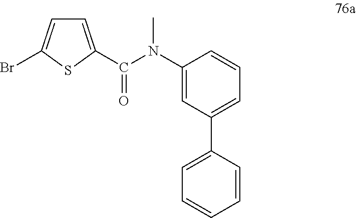 [SMILES: CN(C(=O)c1ccc(Br)s1)c1cccc(-c2ccccc2)c1]

The title compound was prepared from 5-bromothiophene-2-carbonyl chloride (62 mg, 0.27 mmol) and compound N-methylbiphenyl-3-amine (76b) (50 mg, 0.27 mmol), yielded as colorless solid (100 mg, 98%); MS (ESI): 372 (M+H)⁺; ¹H NMR (CD₃COCD₃): 3.43 (s, 3H), 6.57 (d, J=4.1 Hz, 1H), 6.92 (d, J=4.1 Hz, 1H), 7.36-7.41 (m, 2H), 7.47 (t, J=7.9 Hz, 2H), 7.59 (t, J=7.9 Hz, 1H), 7.67-7.70 (m, 2H), 7.72 (t, J=2.0 Hz, 1H), 7.76 (ddd, J=7.9, 2.0, 0.9 Hz, 1H); ¹³C NMR (CD₃COCD₃): 39.0, 118.3, 127.6, 127.7, 127.8, 127.9, 128.8, 129.8, 131.0, 131.3, 133.3, 140.5, 141.2, 143.7, 145.3, 161.3.

### N-(Biphenyl-3-yl)-5-(3-methoxyphenyl)-N-methylthiophene-2-carboxamide (76)

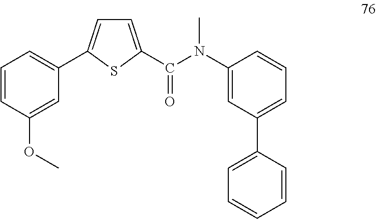 [SMILES: COc1cccc(-c2ccc(C(=O)N(C)c3cccc(-c4ccccc4)c3)s2)c1]

The title compound was prepared by reaction of N-(biphenyl-3-yl)-5-bromo-N-methylthiophene-2-carboxamide (76a) (37 mg, 0.1 mmol) and 3-methoxyphenylboronic acid (18 mg, 0.12 mmol) with tetrakis(triphenylphosphine) palladium (12 mg, 0.01 mmol) as catalyst according to method B for 14 h. Purification by FC (n-hexane/ethyl acetate 8:1) afforded the desired compound as colorless solid (38 mg, 95%); MS (ESI): 400 (M+H)⁺; ¹H NMR (CD₃COCD₃): 3.46 (s, 3H), 3.81 (s, 3H), 6.65 (d, J=4.0 Hz, 1H), 6.89 (dd, J=8.0, 2.1 Hz, 1H), 7.09 (s, 1H), 7.12 (d, J=7.6 Hz, 1H), 7.15 (d, J=4.0 Hz, 1H), 7.28 (t, J=8.0 Hz, 1H), 7.36-7.39 (m, 2H), 7.46 (t, J=7.8 Hz, 2H), 7.57 (t, J=7.8 Hz, 1H), 7.68 (d, J=8.0 Hz, 2H), 7.71-7.74 (m, 2H); ¹³C NMR (CD₃COCD₃): 39.0, 55.6, 112.0, 114.9, 119.0, 124.1, 127.3, 127.5, 127.7, 127.8, 128.7, 129.8, 131.0, 131.2, 133.3, 135.7, 138.8, 140.7, 143.6, 146.1, 149.0, 161.2, 162.5.

### 1.77. 5-(2-Fluoro-3-methoxyphenyl)-N-(3-methoxyphenyl)thiophene-2-carboxamide (77)

### 5-Bromo-N-(3-methoxyphenyl)thiophene-2-carboxamide (77a)

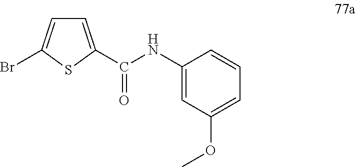 [SMILES: COc1cccc(NC(=O)c2ccc(Br)s2)c1]

The title compound was prepared by reaction of 5-bromothiophene-2-carbonyl chloride (113 mg, 0.5 mmol) and 3-methoxylaniline (62 mg, 0.5 mmol) in presence of triethylamine (0.08 ml, 0.58 mmol) according to method A. Purification by FC (n-hexane/ethyl acetate 10:1→6:1) yielded the product as colorless solid (150 mg, 96%); MS (ESI): 313 (M+H)⁺; ¹H NMR (CD₃COCD₃): 3.79 (s, 3H), 6.70 (d, J=8.0 Hz, 1H), 7.22-7.25 (m, 2H), 7.28 (d, J=8.0 Hz, 1H), 7.47 (s, 1H), 7.71 (d, J=4.1 Hz, 1H), 9.49 (br s, 1H); ¹³C NMR (CD₃COCD₃): 55.5, 106.8, 110.4, 113.2, 118.8, 129.7, 130.4, 132.2, 140.8, 143.4, 159.7, 161.1.

### 5-(2-Fluoro-3-methoxyphenyl)-N-(3-methoxyphenyl)thiophene-2-carboxamide (77)

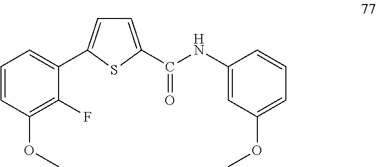 [SMILES: COc1cccc(NC(=O)c2ccc(-c3cccc(OC)c3F)s2)c1]

The title compound was prepared by reaction of 5-bromo-N-(3-methoxyphenyl)thiophene-2-carboxamide (77a) (62 mg, 0.2 mmol) and 2-fluoro-3-methoxyphenylboronic acid (38 mg, 0.24 mmol) with tetrakis(triphenylphosphine) palladium (23 mg, 0.02 mmol) as catalyst according to method B for 14 h. Purification by FC (n-hexane/ethyl acetate 6:1) afforded the desired compound as colorless solid (60 mg, 84%); MS (ESI): 358 (M+H)⁺; ¹H NMR (CD₃COCD₃): 3.80 (s, 3H), 3.94 (s, 3H), 6.70 (ddd, J=8.2, 2.5, 0.9 Hz, 1H), 7.17 (td, J=8.2, 1.6 Hz, 1H), 7.21 (td, J=7.6, 0.9 Hz, 1H), 7.24 (t, J=8.2 Hz, 1H), 7.32-7.36 (m, 2H), 7.53 (t, J=2.2 Hz, 1H), 7.57 (dd, J=4.1, 1.0, Hz, 1H), 7.91 (dd, J=4.1, 1.0 Hz, 1H), 9.52 (br s, 1H); ¹³C NMR (CD₃COCD₃): 55.5, 56.8, 106.8, 110.3, 113.2, 114.3, 118.6, 120.6, 122.7, 122.8, 125.6, 127.9, 128.0, 129.5, 130.3, 141.1, 141.3, 141.4, 142.6, 149.0, 149.6, 149.7, 150.9, 160.6, 161.1.

### 1.78. 5-(2-Fluoro-3-methoxyphenyl)-N-(3-methoxyphenyl)-N-phenylthiophene-2-carboxamide (78)

### 5-Bromo-N-(3-methoxyphenyl)-N-phenylthiophene-2-carboxamide (78a)

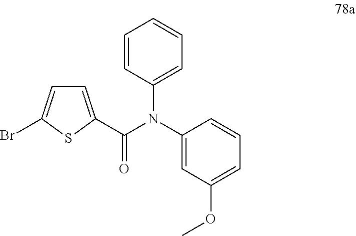 [SMILES: COc1cccc(N(C(=O)c2ccc(Br)s2)c2ccccc2)c1]

The title compound was prepared by reaction of 5-bromothiophene-2-carbonyl chloride (225 mg, 1 mmol) and 3-methoxy-N-phenylaniline (199 mg, 1 mmol) in presence of triethylamine (0.08 ml, 0.58 mmol) according to method A. Purification by FC (n-hexane/ethyl acetate 6:1) yielded the product as colorless oil (220 mg, 57%); MS (ESI): 388 (M+H)⁺; ¹H NMR (CD₃COCD₃): 3.79 (s, 3H), 6.67 (d, J=4.1 Hz, 1H), 6.93 (ddd, J=8.5, 2.0, 0.9 Hz, 1H), 6.96 (ddd, J=7.9, 2.0, 0.9 Hz, 1H), 6.99 (d, J=4.1 Hz, 1H), 7.01 (t, J=2.0 Hz, 1H), 7.32-7.36 (m, 2H), 7.39-7.45 (m, 4H); ¹³C NMR (CD₃COCD₃): 55.8, 113.9, 115.0, 119.1, 121.2, 128.2, 128.9, 130.2, 131.0, 131.2, 134.0, 141.6, 144.2, 145.2, 161.5.

### 5-(2-Fluoro-3-methoxyphenyl)-N-(3-methoxyphenyl)-N-phenylthiophene-2-carboxamide (78)

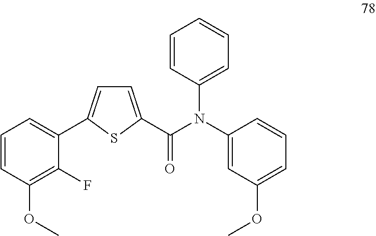 [SMILES: COc1cccc(N(C(=O)c2ccc(-c3cccc(OC)c3F)s2)c2ccccc2)c1]

The title compound was prepared by reaction of 5-bromo-N-(3-methoxyphenyl)-N-phenylthiophene-2-carboxamide (78a) (39 mg, 0.1 mmol) and 2-fluoro-3-methoxyphenylboronic acid (20 mg, 0.12 mmol) with tetrakis(triphenylphosphine) palladium (12 mg, 0.01 mmol) as catalyst according to method B for 14 h. Purification by FC (n-hexane/ethyl acetate 6:1) afforded the desired compound as colorless solid (30 mg, 69%); MS (ESI): 434 (M+H)⁺; ¹H NMR (CD₃COCD₃): 3.78 (s, 3H), 3.91 (s, 3H), 6.82 (dd, J=4.1, 1.0 Hz, 1H), 6.92 (ddd, J=8.5, 2.5, 0.9 Hz, 1H), 6.96 (ddd, J=8.0, 2.0, 1.0 Hz, 1H), 7.01 (t, J=2.0 Hz, 1H), 7.13 (dt, J=8.0, 2.0 Hz, 1H), 7.16 (dt, J=8.0, 1.0 Hz, 1H), 7.20-7.23 (m, 1H), 7.29 (dd, J=4.1, 1.0 Hz, 1H), 7.31-7.35 (m, 2H), 7.39-7.45 (m, 4H); ¹³C NMR (CD₃COCD₃): 55.8, 56.7, 113.6, 114.2, 115.0, 120.4, 120.5, 121.2, 125.5, 125.6, 127.0, 127.1, 127.9, 128.9, 130.1, 130.9, 133.4, 140.2, 140.3, 140.4, 142.4, 144.7, 145.7, 148.9, 149.5, 149.6, 150.9, 161.5, 162.7.

### 1.79. N-(3-Methoxyphenyl)-N-methyl-2-(3-methylphenyl)-1,3-thiazole-5-carboxamide (79)

### 2-Bromo-N-(3-methoxyphenyl)-N-methyl-1,3-thiazole-5-carboxamide (79a)

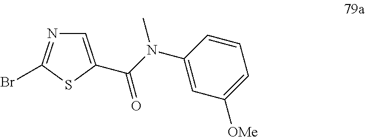 [SMILES: COc1cccc(N(C)C(=O)c2cnc(Br)s2)c1]

A solution of 2-bromo-1,3-thiazole-5-carboxylic acid (416 mg, 2 mmol), thionyl chloride (0.58 ml, 4 mmol), 5 drops of DMF in dry toluene (10 ml) was heated at 110° C. for 4 hours. After cooling, the solvent was removed under reduced pressure. This residue was diluted in dry CH₂Cl₂ and 3-methoxy-N-methylaniline (0.26 ml, 2 mmol) was added followed by triethylamine (0.28 ml, 2 mmol) according to method A and the solution was stirred at rt overnight under N₂ atmosphere. Purification on silica gel (n-hexane/ethyl acetate 7:3) afforded the desired product as pale brown solid (393 mg, 60%); MS (ESI): 328 (M+H)⁺; mp: 106° C.; IR (cm⁻¹): 3098, 3059, 3014, 2966, 2943, 2838, 1633, 1582; ¹H NMR (CD₃COCD₃): 3.67 (s, 3H), 3.85 (s, 3H), 6.99 (ddd, J=0.9, 1.95, 7.7 Hz, 1H), 7.05 (t, J=2.3 Hz, 1H), 7.09 (ddd, J=0.85, 2.5, 8.4 Hz, 1H), 7.18 (s, 1H), 7.42-7.46 (m, 1H); ¹³C NMR (CD₃COCD₃): 38.6, 56.0, 114.9, 115.8, 121.3, 131.8, 136.8, 144.9, 145.7, 155.6, 159.9, 162.1.

### N-(3-Methoxyphenyl)-N-methyl-2-(3-methylphenyl)-1,3-thiazole-5-carboxamide (79)

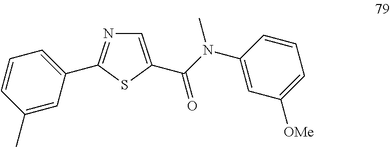 [SMILES: COc1cccc(N(C)C(=O)c2cnc(-c3cccc(C)c3)s2)c1]

A solution of 2-bromo-N-(3-methoxyphenyl)-N-methyl-1,3-thiazole-5-carboxamide (79a) (200 mg, 0.61 mmol), m-tolyl boronic acid (105 mg, 0.77 mmol), caesium carbonate (596 mg, 1.83 mmol) and tetrakis(triphenylphosphine) palladium (14 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified on silica gel (n-hexane/ethyl acetate 7:3) and afforded the desired product as beige solid (170 mg, 82%); MS (ESI): 339 (M+H)⁺; mp: 88° C.; IR (cm⁻¹): 3065, 3005, 2924, 2841, 1626, 1588; ¹H NMR (CD₃COCD₃): 2.36 (s, 3H), 3.40 (s, 3H), 3.83 (s, 3H), 6.98-7.00 (m, 1H), 7.04-7.06 (m, 2H), 7.28-7.30 (m, 2H), 7.32-7.35 (m, 1H), 7.42 (dt, J=0.7, 7.75 Hz, 1H), 7.64-6.66 (m, 1H), 7.70-7.71 (m, 1H); ¹³C NMR (CD₃COCD₃): 21.2, 38.7, 55.9, 114.7, 115.2, 121.1, 124.5, 127.7, 129.9, 131.6, 132.4, 134.0, 134.7, 139.8, 145.8, 147.6, 161.2, 161.9, 171.6.

### 1.80. 2-(2-Fluoro-3-methoxyphenyl)-N-(3-hydroxybenzyl)-N-methyl-1,3-thiazole-5-carboxamide (80)

### 2-Bromo-N-(3-methoxybenzyl)-N-methyl-1,3-thiazole-5-carboxamide (80b)

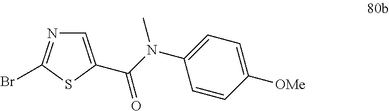 [SMILES: COc1ccc(N(C)C(=O)c2cnc(Br)s2)cc1]

A solution of 2-bromo-1,3-thiazole-5-carboxylic acid (416 mg, 2 mmol), thionyl chloride (0.58 ml, 4 mmol), 5 drops of DMF in dry toluene (10 ml) was heated at 110° C. for 4 h. After cooling, the solvent was removed under reduced pressure. This residue was diluted in dry CH₂Cl₂ and 3-methoxy-N-methylbenzylamine (0.30 ml, 2 mmol) was added followed by triethylamine (0.28 ml, 2 mmol) according method A. The solution was stirred at rt overnight under N₂ atmosphere. Purification on silica gel (n-hexane/ethyl acetate 7:3) afforded the desired product as yellow oil (530 mg, 78%); MS (ESI): 342 (M+H)⁺; IR (cm⁻¹): 2934, 2835, 1611; ¹H NMR (CD₃COCD₃): 2.98-3.34 (m, 3H), 3.79 (s, 3H), 4.68-4.82 (m, 2H), 6.86-6.93 (m, 3H), 7.63-8.14 (m, 1H); ¹³C NMR (CD₃COCD₃): 37.1, 52.4, 55.5, 113.7, 114.5, 119.4, 120.9, 130.7, 139.5, 143.2, 154.7, 161.1, 161.5.

### 2-Bromo-N-(3-hydroxybenzyl)-N-methyl-1,3-thiazole-5-carboxamide (80a)

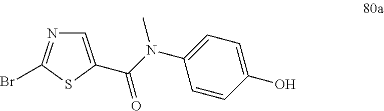 [SMILES: CN(C(=O)c1cnc(Br)s1)c1ccc(O)cc1]

The title compound was prepared by reaction of 2-bromo-N-(3-methoxybenzyl)-N-methyl-1,3-thiazole-5-carboxamide (80b) (530 mg, 1.55 mmol) with borontrifluoride dimethyl sulphide complex (0.98 ml, 9.32 mmol) according to method C for 2 h at 0° C. and 1 h at rt. The product was purified on MP-LC (n-hexane/ethyl acetate 100:0 to 80:20, 15 min, 80:20 to 60:40, 35 min) and afforded the desired product as pale red oil (236 mg, 47%); MS (ESI): 328 (M+H)⁺; IR (cm⁻¹): 3286, 3101, 2969, 2928, 2871, 1731, 1702, 1603; ¹H NMR (CD₃COCD₃): 2.99-3.34 (m, 3H), 4.61-4.82 (m, 2H), 6.72-6.92 (m, 3H), 7.60-8.13 (m, 1H), 8.40-8.55 (br s, 1H).

### 2-(2-Fluoro-3-methoxyphenyl)-N-(3-hydroxybenzyl)-N-methyl-1,3-thiazole-5-carboxamide (80)

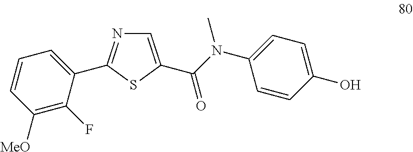 [SMILES: COc1cccc(-c2ncc(C(=O)N(C)c3ccc(O)cc3)s2)c1F]

A solution of 2-bromo-N-(3-hydroxybenzyl)-N-methyl-1,3-thiazole-5-carboxamide (80a) (104 mg, 0.32 mmol), 2-fluoro-3-methoxyphenyl boronic acid (81 mg, 0.48 mmol), caesium carbonate (311 mg, 0.95 mmol) and tetrakis(triphenylphosphine) palladium (7 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified by MP-LC (n-hexane/ethyl acetate 100:0 to 60:40, 60 min). Trituration of the residue in a little amount of cold acetonitrile afforded the desired product as beige solid (83 mg, 70%); MS (ESI): 373 (M+H)⁺; mp: 140° C.; IR (cm⁻¹): 3220, 3029, 2993, 2931, 2838, 1737, 1591; ¹H NMR (CD₃COCD₃): 2.82 (s, 1H), 3.2 (br s, 3H), 3.97 (s, 3H), 4.76 (br s, 2H), 6.77-6.85 (m, 3H), 7.22 (t, J=7.55 Hz, 1H), 7.26-7.32 (m, 2H), 7.82-7.87 (br s, 1H), 8.33-8.40 (br s, 1H); ¹³C NMR (CD₃COCD₃): 56.9, 104.4, 115.3, 115.4, 116.12, 116.14, 120.1, 122.26, 122.35, 125.57, 125.60, 139.7, 149.3, 149.4, 150.0, 152.0, 158.8, 162.9.

### 1.81. N-(3-Methoxyphenyl)-N-methyl-5-(3-methylphenyl)-1,3-thiazole-2-carboxamide (81)

### 5-(3-Methylphenyl)-1,3-thiazole (81b)

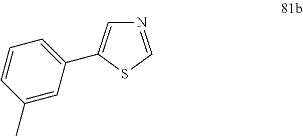

A solution of 5-bromo-1,3-thiazole (1 g, 6.10 mmol), m-tolyl boronic acid (1.244 g, 9.14 mmol), caesium carbonate (5.962 g, 18.30 mmol) and tetrakis(triphenylphosphine) palladium (140 mg, 0.02 eq) in oxygen free DME/EtOH/water (1:1:1) (6 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified on silica gel (n-hexane/ethyl acetate 7:3) and afforded the desired product as colourless oil (1.06 g, 99%); MS (ESI): 176 (M+H)⁺; IR (cm⁻¹): 3086, 3026, 2957, 2922, 2862, 1605; ¹H NMR (CD₃COCD₃): 2.37 (s, 3H), 7.17-7.20 (m, 1H), 7.32 (t, J=7.7 Hz, 1H), 7.45-7.48 (m, 1H), 7.50-7.52 (m, 1H), 8.19 (s, 1H), 8.93 (s, 1H); ¹³C NMR (CD₃COCD₃): 21.4, 124.8, 128.3, 129.99, 130.01, 132.1, 139.8, 140.1, 153.2.

### Lithium 5-(3-methylphenyl)-1,3-thiazole-2-carboxylate (81a)

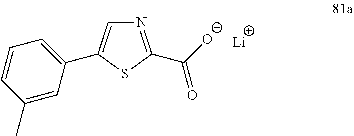

To a solution of 5-(3-methylphenyl)-1,3-thiazole (81b) (1.05 g, 6 mmol) in dry THF (50 ml) was added drop wise n-BuLi (2.88 ml, 7.21 mmol, 2.5M in hexane) at −78° C. under N₂ atmosphere. The reaction mixture was kept at −78° C. for 1 h and warmed up to −5° C. over 3 h. The reaction mixture was cooled at −78° C. and dry carbon dioxide gas was bubbled through the solution for 3 h. The reaction mixture was warmed up to rt keeping CO₂ gas bubbling overnight. Solvent was removed under reduced pressure (bath temperature of rotavapor at 20° C.). The residue was triturated with diethylether and filtered to afford the desired compound as a pale brown solid (977 mg, 72%); MS (ESI): 220 (M+H)⁺; IR (cm⁻¹): 3301, 3095, 3032, 2960, 2922, 2868, 1737, 1618; ¹H NMR (CD₃SOCD₃): 2.34 (s, 3H), 7.16 (d, J=7.2 Hz, 1H), 7.31 (t, J=7.45 Hz, 1H), 7.46 (d, J=7.2 Hz, 1H), 7.50 (s, 1H), 8.12 (s, 1H); ¹³C NMR (CD₃SOCD₃): 20.9, 123.6, 126.9, 128.99, 129.06, 131.2, 138.5, 141.0, 161.7, 170.3.

### N-(3-Methoxyphenyl)-N-methyl-5-(3-methylphenyl)-1,3-thiazole-2-carboxamide (81)

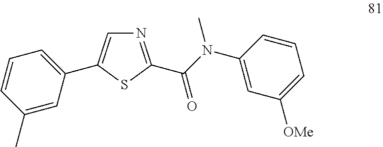 [SMILES: COc1cccc(N(C)C(=O)c2ncc(-c3cccc(C)c3)s2)c1]

To a solution of lithium 5-(3-methylphenyl)-1,3-thiazole-2-carboxylate (81a) (450 mg, 2 mmol) in CH₂Cl₂ (15 ml) was added drop wise oxalyl chloride (0.34 ml, 4 mmol) followed by few drops of DMF at 0° C. under N₂ atmosphere. The reaction mixture was stirred at 0° C. for 10 min and at rt for 3 h. Then, the solvent was removed under reduced pressure (bath temperature of rotavapor at 20° C.). The residue was diluted in dry CH₂Cl₂ and 3-methoxy-N-methylaniline (0.26 ml, 2 mmol) was added followed by triethylamine (0.28 ml, 2 mmol). The solution was stirred at rt overnight under N₂ atmosphere. The solvent was removed under reduced pressure (bath temperature of rotavapor at 20° C.). Aqueous Na₂CO₃ 2N (15 ml) was added followed by ethyl acetate (15 ml). The aqueous layer was extracted with ethyl acetate. The organic layer was washed twice with Na₂CO₃ 2N (15 ml), once with water (15 ml), dried over MgSO₄, filtered and concentrated under reduced pressure. Purification on silica gel (n-hexane/ethyl acetate 7:3) afforded the desired product as yellow solid (520 mg, 77%); MS (ESI): 339 (M+H)⁺; mp: 85° C.; IR (cm⁻¹): 3095, 3014, 2975, 2919, 2838, 1746, 1633, 1588; ¹H NMR (CD₃COCD₃): 2.36 (s, 3H), 3.54 (s, 2H), 3.78 (s, 3H), 6.87 (ddd, J=0.6, 2.5, 8.25 Hz, 2H), 6.92 (t, J=2.1 Hz, 1H), 7.20-7.22 (m, 1H), 7.25-7.29 (m, 1H), 7.30-7.34 (m, 1H), 7.43-7.47 (m, 1H), 7.48-7.50 (m, 1H), 7.91-7.96 (m, 1H); ¹³C NMR (CD₃COCD₃): 21.3, 39.5, 55.7, 113.6, 113.8, 120.1, 124.87, 124.93, 128.3, 128.4, 130.1, 130.55, 130.62, 131.5, 139.7, 139.9, 144.3, 146.6, 161.2.

### 1.82. N-Cyclopropyl-N-(3-methoxyphenyl)-5-(3-methylphenyl)thiophene-2-carboxamide (82)

### 5-Bromo-N-cyclopropyl-N-(3-methoxyphenyl)thiophene-2-carboxamide (82a)

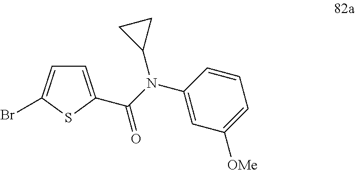 [SMILES: COc1cccc(N(C(=O)c2ccc(Br)s2)C2CC2)c1]

In a sealed tube was introduced 1-bromo-3-methoxybenzene (374 mg, 2 mmol), sodium tert-butoxide (288 mg, 3 mmol), 2,2′-bis(diphenylphosphino)-1,1′-binaphthyl (38 mg, 0.06 mmol), cyclopropylamine (183 mg, 3.2 mmol), tris(dibenzylideneacetone)dipalladium(0) (18 mg, 0.02 mmol) and dry toluene (2-3 ml). The tube was flushed few seconds with N₂, closed and wrapped with aluminum foil. The reaction mixture was heated at 80° C. overnight. After cooling, diethyl ether was added to quench the reaction and the solution was filtered on celite. The solvent was removed under reduced pressure and the product was used in the next step without further purification.

To a solution of freshly prepared 5-bromothiophene-2-carbonyl chloride (2 mmol) (prepared from 5-bromothiophene-2-carboxylic acid (414 mg, 2 mmol), thionyl chloride (0.58 ml, 4 mmol) in dry toluene (10 ml) and catalytic amount of DMF) was added the freshly synthesized N-cyclopropyl-3-methoxyaniline (2 mmol) diluted in little amount of dry CH₂Cl₂, followed by triethylamine (0.28 ml, 2 mmol) according to method A for 24 h. Purification on silica gel (n-hexane/ethyl acetate 7:3) afforded the desired product as yellow solid (343 mg, 49%); MS (ESI): 353 (M+H)⁺; mp: 60° C.; IR (cm⁻¹): 3086, 3014, 2946, 2841, 1740, 1630, 1597; ¹H NMR (CD₃COCD₃): 0.58-0.62 (m, 2H), 0.78-0.82 (m, 2H), 3.28-3.32 (m, 1H), 3.82 (s, 3H), 6.66 (d, J=4.1 Hz, 1H), 6.86 (ddd, J=0.95, 1.9, 7.85 Hz, 1H), 6.91 (t, J=2.3 Hz, 1H), 6.95 (d, J=4.1 Hz, 1H), 7.03 (ddd, J=0.9, 2.6, 8.45 Hz, 1H), 7.36-7.39 (m, 1H); ¹³C NMR (CD₃COCD₃): 7.8, 33.1, 55.9, 115.1, 116.0, 118.5, 122.5, 131.1, 132.9, 142.0, 143.3, 161.6, 162.6.

### N-Cyclopropyl-N-(3-methoxyphenyl)-5-(3-methylphenyl)thiophene-2-carboxamide (82)

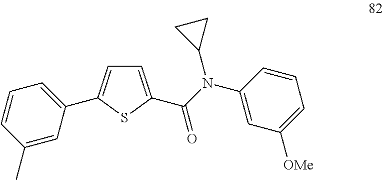 [SMILES: COc1cccc(N(C(=O)c2ccc(-c3cccc(C)c3)s2)C2CC2)c1]

A solution of 5-bromo-N-cyclopropyl-N-(3-methoxyphenyl)thiophene-2-carboxamide (82a) (205 mg, 0.58 mmol), m-tolyl boronic acid (118 mg, 0.87 mmol), caesium carbonate (570 mg, 1.75 mmol) and tetrakis(triphenylphosphine) palladium (14 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified on MP-LC (n-hexane/ethyl acetate 100:0 to 80:20, 10 min, 80:20 to 60:50, 45 min) and afforded the desired product as yellow solid (181 mg, 86%); MS (ESI): 364 (M+H)⁺; mp: 68° C.; IR (cm⁻¹): 3092, 3014, 2972, 2922, 2835, 1737, 1702, 1627, 1600; ¹H NMR (CD₃COCD₃): 0.61-0.64 (m, 2H), 0.79-0.83 (m, 2H), 2.33 (s, 3H), 3.31-3.35 (m, 1H), 3.81 (s, 3H), 6.73 (d, J=4 Hz, 1H), 6.88 (ddd, J=0.9, 1.95, 7.7 Hz, 1H), 6.92 (t, J=2.3 Hz, 1H), 6.99 (ddd, J=0.9, 2.55, 8.45 Hz, 1H), 7.13-7.14 (m, 1H), 7.15 (d, J=4 Hz, 1H), 7.25-7.28 (m, 1H), 7.34-7.38 (m, 1H), 7.37-7.39 (m, 1H), 7.42-7.43 (m, 1H); ¹³C NMR (CD₃COCD₃): 8.1, 21.3, 33.2, 55.8, 114.5, 115.8, 122.3, 123.7, 123.8, 127.2, 129.8, 129.9, 130.9, 132.9, 134.4, 139.5, 139.6, 144.0, 149.3, 161.5, 163.7.

### 1.83. N-Cyclopropyl-5-(2-fluoro-3-methoxyphenyl)-N-(3-hydroxybenzyl)thiophene-2-carboxamide (83)

### 5-Bromo-N-cyclopropyl-N-(3-methoxybenzyl)thiophene-2-carboxamide (83b)

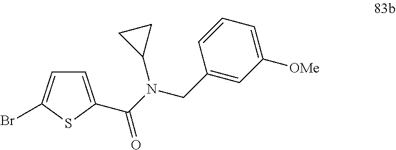 [SMILES: COc1cccc(CN(C(=O)c2ccc(Br)s2)C2CC2)c1]

In a sealed tube was introduced 1-(bromomethyl)-3-methoxybenzene (402 mg, 2 mmol), sodium tert-butoxide (288 mg, 3 mmol), 2,2′-bis(diphenylphosphino)-1,1′-binaphthyl (38 mg, 0.06 mmol), cyclopropylamine (183 mg, 3.2 mmol), tris(dibenzylideneacetone)dipalladium(0) (18 mg, 0.02 mmol) and dry toluene (2-3 ml). The tube was flushed few seconds with N₂, closed and wrapped with aluminum foil. The reaction mixture was heated at 80° C. overnight. After cooling, diethyl ether was added to quench the reaction and the solution was filtered on celite. The solvent was removed under reduced pressure and the product was used for the next step without further purification.

To a solution of freshly prepared 5-bromothiophene-2-carbonyl chloride (2 mmol) (prepared from 5-bromothiophene-2-carboxylic acid (414 mg, 2 mmol), thionyl chloride (0.58 ml, 4 mmol) in dry toluene (10 ml) and catalytic amount of DMF) was added the freshly synthesized N-(3-methoxybenzyl)cyclopropanamine (2 mmol) diluted in little amount of dry CH₂Cl₂, followed by triethylamine (0.28 ml, 2 mmol) according to method A for 24 h. Purification on silica gel (n-hexane/ethyl acetate 7:3) afforded the desired product as yellow oil (302 mg, 41%); MS (ESI): 367 (M+H)⁺; IR (cm⁻¹): 3089, 3008, 2946, 2838, 1737, 1600; ¹H NMR (CD₃COCD₃): 0.78-0.81 (m, 2H), 0.88-0.92 (m, 2H), 2.98-3.04 (m, 1H), 3.77 (s, 3H), 4.74 (s, 2H), 6.82-6.85 (m, 1H), 6.88-6.91 (m, 2H), 7.18 (d, J=4.05 Hz, 1H), 7.23-7.27 (m, 1H), 7.61 (d, J=4.05 Hz, 1H); ¹³C NMR (CD₃COCD₃): 11.3, 32.0, 51.9, 55.4, 113.2, 114.1, 117.9, 120.5, 130.4, 131.5, 132.3, 141.0, 142.5, 160.9, 164.1.

### 5-Bromo-N-cyclopropyl-N-(3-hydroxybenzyl)thiophene-2-carboxamide (83a)

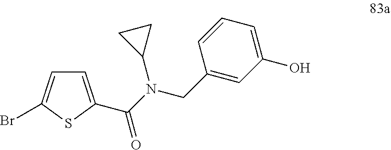 [SMILES: O=C(c1ccc(Br)s1)N(Cc1cccc(O)c1)C1CC1]

The title compound was prepared by reaction of 5-bromo-N-cyclopropyl-N-(3-methoxybenzyl)thiophene-2-carboxamide (83b) (278 mg, 0.76 mmol) with borontrifluoride dimethyl sulphide complex (0.48 ml, 4.56 mmol) according to method C for 2 h at 0° C. and overnight at rt. The product as brown solid (270 mg, 100%) was analytically pure and used in the next step without further purification; MS (ESI): 353 (M+H)⁺; mp: 128° C.; IR (cm⁻¹): 3268, 3098, 3002, 2975, 2928, 2429, 1740, 1603, 1570; ¹H NMR (CD₃COCD₃): 0.77-0.81 (m, 2H), 0.87-0.92 (m, 2H), 2.96-3.02 (m, 1H), 4.70 (s, 2H), 6.73 (ddd, J=0.85, 2.65, 7.8 Hz, 1H), 6.78-6.80 (m, 2H), 7.13-7.17 (m, 1H), 7.18 (d, J=4.05 Hz, 1H), 7.60 (d, J=4.05 Hz, 1H), 8.28 (br s, 1H); ¹³C NMR (CD₃COCD₃): 11.3, 31.9, 51.9, 114.9, 115.1, 117.9, 119.5, 130.4, 131.5, 132.3, 140.9, 142.5, 158.5, 164.1.

### N-Cyclopropyl-5-(2-fluoro-3-methoxyphenyl)-N-(3-hydroxybenzyl)thiophene-2-carboxamide (83)

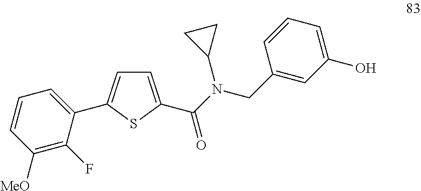 [SMILES: COc1cccc(-c2ccc(C(=O)N(Cc3cccc(O)c3)C3CC3)s2)c1F]

A solution of 5-bromo-N-cyclopropyl-N-(3-hydroxybenzyl)thiophene-2-carboxamide (83a) (104 mg, 0.32 mmol), 2-fluoro-3-methoxyphenyl boronic acid (81 mg, 0.48 mmol), cesium carbonate (311 mg, 0.95 mmol) and tetrakis(triphenylphosphine) palladium (7 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified by HPLC preparative (acetonitrile/water 40:60 to 100:0, solvent containing 1 ml of TFA for 1 l of solvent) and afforded the desired product as beige solid (41 mg, 37%); MS (ESI): 398 (M+H)⁺; mp: 150° C.; IR (cm⁻¹): 3253, 3008, 2972, 2937, 2841, 2426, 1740, 1600, 1570; ¹H NMR (CD₃COCD₃): 0.77-0.80 (m, 2H), 0.87-0.92 (m, 2H), 3.00-3.06 (m, 1H), 3.94 (s, 3H), 4.74 (s, 2H), 6.73-6.76 (m, 1H), 6.81-6.84 (m, 2H), 7.13-7.22 (m, 3H), 7.32-7.35 (m, 1H), 7.52 (dd, J=1.0, 4.05 Hz, 1H), 7.78 (m, 1H); ¹³C NMR (CD₃COCD₃): 11.2, 32.1, 51.9, 56.7, 114.07, 114.08, 114.9, 115.1, 119.5, 120.57, 120.58, 122.8, 122.9, 125.5, 125.6, 127.2, 127.3, 130.4, 131.8, 140.67, 140.71, 141.1, 141.29, 141.31, 148.9, 149.6, 149.7, 150.9, 158.5, 165.1.

### 1.84. N-(3-Methoxyphenyl)-N-methyl-5-(3-methylphenyl)thiophene-2-sulfonamide (84)

### 5-Bromo-N-(3-methoxyphenyl)-N-methylthiophene-2-sulfonamide (84a)

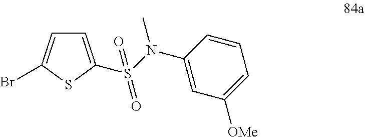 [SMILES: COc1cccc(N(C)S(=O)(=O)c2ccc(Br)s2)c1]

To a solution of 3-methoxy-N-methylaniline (137 mg, 1 mmol) in CH₂Cl₂ (3 ml) was added NaOH 50% (1 ml) under stirring followed by tetrabutyl ammonium hydrogen sulphate (51 mg, 0.15 mmol). After few minutes, 5-bromothiophene-2-sulfonyl chloride (262 mg, 1 mmol) was added to the reaction mixture. The solution was stirred at rt for 5 h, then water (10 ml) was added to quench the reaction followed by ethyl acetate (10 ml). The aqueous layer was extracted twice with ethyl acetate. The organic layer was dried over MgSO₄, filtered and concentrated under reduced pressure. The product was purified on silica gel (n-hexane/ethyl acetate 8:2) and afforded the desired compound as yellowish oil (277 mg, 77%); IR (cm⁻¹): 3101, 2938, 2835, 1734, 1602, 1354; ¹H NMR (CD₃COCD₃): 3.27 (s, 3H), 3.77 (s, 3H), 6.78-6.81 (m, 2H), 6.90-6.92 (m, 1H), 7.26-7.30 (m, 2H), 7.31 (d, J=4.1 Hz, 1H); ¹³C NMR (CD₃COCD₃): 38.7, 55.8, 113.5, 114.2, 119.3, 120.1, 130.5, 132.2, 134.3, 138.9, 143.2, 161.0.

### N-(3-Methoxyphenyl)-N-methyl-5-(3-methylphenyl)thiophene-2-sulfonamide (84)

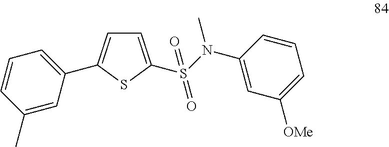 [SMILES: COc1cccc(N(C)S(=O)(=O)c2ccc(-c3cccc(C)c3)s2)c1]

A solution of 5-bromo-N-(3-methoxyphenyl)-N-methylthiophene-2-sulfonamide (84) (142 mg, 0.39 mmol), m-tolyl boronic acid (80 mg, 0.59 mmol), caesium carbonate (385 mg, 1.18 mmol) and tetrakis(triphenylphosphine) palladium (10 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified on silica gel (n-hexane/ethyl acetate 8:2) and afforded the desired product as yellow oil (147 mg, 99%); MS (ESI): 374 (M+H)⁺; IR (cm⁻¹): 3101, 3008, 2925, 2838, 1708, 1702, 1602; ¹H NMR (CD₃COCD₃): 2.38 (s, 3H), 3.29 (s, 3H), 3.75 (s, 3H), 6.80 (dd, J=0.9, 2.15 Hz, 1H), 6.81-6.82 (m, 1H), 6.89 (ddd, J=0.8, 2.5, 8.4 Hz, 1H), 7.23-7.28 (m, 2H), 7.33-7.36 (m, 1H), 7.39 (d, J=4.0 Hz, 1H), 7.48-7.49 (m, 1H), 7.50 (d, J=4.0 Hz, 1H), 7.53-7.54 (m, 1H); ¹³C NMR (CD₃COCD₃): 21.3, 38.8, 55.7, 113.5, 114.0, 119.3, 124.1, 124.3, 127.6, 130.1, 130.4, 130.8, 133.5, 134.7, 135.9, 140.0, 143.6, 152.0, 160.9.

### 1.85. 5-(2-Fluoro-3-methoxyphenyl)-N-(3-hydroxybenzyl)-N-methylthiophene-2-sulfonamide (85)

### 5-Bromo-N-(3-methoxybenzyl)-N-methylthiophene-2-sulfonamide (85b)

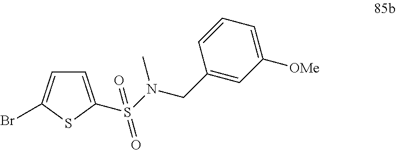 [SMILES: COc1cccc(CN(C)S(=O)(=O)c2ccc(Br)s2)c1]

To a solution of 3-methoxy-N-methylbenzylamine (302 mg, 2 mmol) in CH₂Cl₂ (6 ml) was added NaOH 50% (2 ml) under stirring followed by tetrabutyl ammonium hydrogen sulphate (102 mg, 0.30 mmol). After few minutes, 5-bromothiophene-2-sulfonyl chloride (524 mg, 2 mmol) was added to the reaction mixture. The solution was stirred at rt for 5 h, then water (10 ml) was added to quench the reaction followed by ethyl acetate (10 ml). The aqueous layer was extracted twice with ethyl acetate. The organic layer was dried over MgSO₄, filtered and concentrated under reduced pressure. The product was purified on silica gel (n-hexane/ethyl acetate 8:2) afforded the desired compound as yellowish oil (620 mg, 82%); IR (cm⁻¹): 3101, 2937, 2835, 1740, 1600, 1345; ¹H NMR (CD₃COCD₃): 2.70 (s, 3H), 3.80 (s, 3H), 4.21 (s, 2H), 6.88-6.91 (m, 1H), 6.92-6.95 (m, 2H), 7.28-7.31 (m, 1H), 7.38 (d, J=4.05 Hz, 1H), 7.52 (d, J=4.05 Hz, 1H); ¹³C NMR (CD₃COCD₃): 35.0, 54.7, 55.5, 114.2, 114.7, 119.7, 121.3, 130.6, 132.5, 133.7, 138.3, 140.0, 161.0.

### 5-Bromo-N-(3-hydroxybenzyl)-N-methylthiophene-2-sulfonamide (85a)

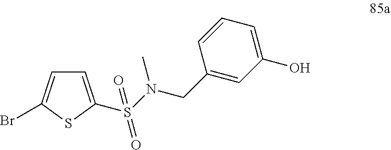 [SMILES: CN(Cc1cccc(O)c1)S(=O)(=O)c1ccc(Br)s1]

The title compound was prepared by reaction of 5-bromo-N-(3-methoxybenzyl)-N-methylthiophene-2-sulfonamide (85a) (528 mg, 1.40 mmol) with borontrifluoride dimethyl sulphide complex (0.88 ml, 8.42 mmol) according to method C overnight at rt. The solvent was removed under reduced pressure and the residue was triturated in water. The precipitate formed was filtered to give the desired product as yellow solid (445 mg, 88%); mp: 106° C.; IR (cm⁻¹): 3438, 3104, 3026, 2981, 2931, 1600, 1337; ¹H NMR (CD₃COCD₃): 2.69 (s, 3H), 4.16 (s, 2H), 6.80 (ddd, J=0.8, 2.55, 8.95 Hz, 1H), 6.81-6.84 (m, 1H), 6.86-6.88 (m, 1H), 7.18-7.21 (m, 1H), 7.38 (d, J=3.95 Hz, 1H), 7.51 (d, J=3.95 Hz, 1H), 8.38 (s, 1H); ¹³C NMR (CD₃COCD₃): 35.0, 54.7, 115.8, 116.0, 119.6, 120.3, 130.6, 132.4, 133.6, 138.3, 140.0, 158.7.

### 5-(2-Fluoro-3-methoxyphenyl)-N-(3-hydroxybenzyl)-N-methylthiophene-2-sulfonamide (85)

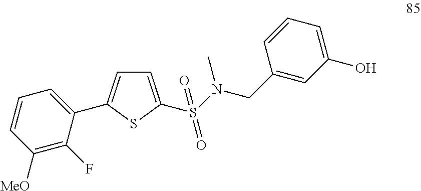 [SMILES: COc1cccc(-c2ccc(S(=O)(=O)N(C)Cc3cccc(O)c3)s2)c1F]

A solution of 5-bromo-N-(3-hydroxybenzyl)-N-methylthiophene-2-sulfonamide (85a) (200 mg, 0.55 mmol), 2-fluoro-3-methoxyphenyl boronic acid (141 mg, 0.83 mmol), caesium carbonate (540 mg, 1.66 mmol) and tetrakis(triphenylphosphine) palladium (13 mg, 0.02 eq) in oxygen free DME/EtOH/water (1:1:1) (3 mL) was heated under microwave irradiation at 150° C. (150 W, 5 bar) for 15 min. The reaction mixture was cooled to rt and quenched with ethyl acetate. The aqueous layer was extracted with ethyl acetate. The combined organic layers were washed with brine, dried over magnesium sulphate, filtered and concentrated under reduced pressure. The residue was purified by HPLC preparative (acetonitrile/water 40:60 to 100:0, solvent containing 1 ml of TFA for 1 l of solvent) and afforded the desired product as colourless oil (42 mg, 19%); MS (ESI): 408 (M+H)⁺; IR (cm⁻¹): 3438, 3107, 3029, 2981, 2928, 2856, 1725, 1600, 1337; ¹H NMR (CD₃COCD₃): 2.72 (s, 3H), 3.95 (s, 3H), 4.19 (s, 2H), 6.80 (ddd, J=0.85, 2.5, 8.25 Hz, 1H), 6.83-6.85 (m, 1H), 6.89-6.90 (m, 1H), 7.17-7.27 (m, 3H), 7.37-7.40 (m, 1H), 7.67 (dd, J=0.55, 4.05 Hz, 1H), 7.71 (dd, J=1.25, 4.0 Hz, 1H); ¹³C NMR (CD₃COCD₃): 35.1, 54.7, 56.8, 114.8, 115.7, 115.9, 120.3, 120.5, 121.9, 125.8, 127.7, 130.5, 133.3, 138.5, 143.7, 148.9, 149.6, 150.9, 158.6.

### Example 2

### Biological Methods

[2,4,6,7-³H]-E1 and [2,4,6,7-³H]-E2 were purchased from Perkin Elmer, Boston. Quickszint Flow 302 scintillator fluid was bought from Zinsser Analytic, Frankfurt. Other chemicals were purchased from Sigma, Roth or Merck.

### 2.1 17β-HSD2 and 17β-HSD1 Enzyme Preparation from Human Placental Enzyme

17β-HSD2 and 17β-HSD1 were obtained from human placenta according to previously described procedures (Kruchten, P. et al., Mol. Cell. Endocrinol., 301: 154-159 (2009)). Fresh human placenta was homogenized. Cytosolic and microsomal fractions were separated by centrifugation at 1000 g, 10000 g and 150000 g. 17β-HSD2 was obtained directly from the microsomal fraction. For the partial purification of 17β-HSD1, the cytosolic fraction was precipitated with ammonium sulfate. Aliquots containing 17β-HSD1 or 17β-HSD2 were stored frozen.

### 2.2 Inhibition of 17β-HSD2 in Cell-Free Assay

Inhibitory activities were evaluated by an established method with minor modifications (Kruchten, P. et al., Mol. Cell. Endocrinol., 301:154-159 (2009)). Briefly, the enzyme preparation was incubated with NAD⁻ [1500 μM] in the presence of potential inhibitors at 37° C. in a phosphate buffer (50 mM) supplemented with 20% of glycerol and EDTA 1 mM. Inhibitor stock solutions were prepared in DMSO. Final concentration of DMSO was adjusted to 1% in all samples. The enzymatic reaction was started by addition of a mixture of unlabelled- and [³H]-E2 (final concentration: 500 nM, 0.11 μCi). After 20 min at 37° C., the incubation was stopped with HgCl₂ and the mixture was extracted with ether. After evaporation, the steroids were dissolved in acetonitrile. E1 and E2 were separated using acetonitrile/water (45:55) as mobile phase in a C18 RP chromatography column (Nucleodur C18, 3 μm, Macherey-Nagel, Duren) connected to a HPLC-system (Agilent 1100 Series, Agilent Technologies, Waldbronn). Detection and quantification of the steroids were performed using a radioflow detector (Berthold Technologies, Bad Wildbad). The conversion rate was calculated according to the following equation:

$$\% conversion = \frac{\% E 1}{\% E 1 + \% E 2} \cdot 100.$$

Each value was calculated from at least three independent experiments.

### 2.3 Inhibition of 17β-HSD1 in Cell-Free Assay

The 17β-HSD1 inhibition assay was performed similarly to the 17β-HSD2 test. The cytosolic fraction was incubated with NADH [500 μM], test compound and a mixture of unlabelled- and [³H]-E1 (final concentration: 500 nM, 0.15 μCi) for 10 min at 37° C. Further treatment of the samples and HPLC separation was carried out as mentioned above for 17β-HSD2.

### 2.4 Inhibition of 17β-HSD2 in a Cellular Assay

Cellular 17β-HSD2 activity is measured using the breast cancer cell-line MDA-MB-23 (17β-HSD1 activity negligible). [³H-E2] (200 nM) is used as substrate, and is incubated with the inhibitor for 3.5 h at 37° C. After ether extraction, substrate and product are separated by HPLC and detected with a radioflow detector following the method previously described. Potency is evaluated as percentage of inhibition (inhibitor concentration: 1 μM) as well as IC₅₀ values as previously mentioned in the 17β-HSD2 cell-free assay.

### 2.5 ER Affinity Assay

The binding affinity of selected compounds to the ERα and ERβ was determined according to Zimmermann et al. (Zimmermann, J. et al., 3. Steroid Biochem. Mol. Biol., 94:57-66 (2005)) using recombinant human proteins. Briefly, 0.25 μM of ERα or ERβ, respectively, were incubated with [³H]-E2 (10 nM) and test compound for 1 h at room temperature. The potential inhibitors were dissolved in DMSO (5% final concentration). Evaluation of non-specific-binding was performed with diethylstilbestrol (10 μM). After incubation, ligand-receptor complexes were selectively bound to hydroxyapatite (5 g/60 ml TE-buffer). The formed complex was separated, washed and resuspended in ethanol. For radiodetection, scintillator cocktail (Quickszint 212, Zinsser Analytic, Frankfurt) was added and the samples were measured in a liquid scintillation counter (Rack Beta Primo 1209, Wallac, Turku). From these results the percentage of [³H]-E2 displacement by the compounds was calculated. The plot of % displacement versus compound concentration resulted in sigmoidal binding curves. The compound concentration to displace 50% of the receptor bound [³H]-E2 were determined. Unlabelled E2 was used as a reference. For determination of the relative binding affinity (RBA) the ratio was calculated according to the following equation:

$$RBA [ \% ] = \frac{{IC}_{50} ( E 2 )}{{IC}_{50} ( compound )} \cdot 100.$$

This results in an RBA value of 100% for E2. After the assay was established and validated a modification was made to increase throughput. Compounds were tested at concentration of 1000·IC₅₀(E2). Results were reported as RBA ranges. Compounds with less than 50% displacement of [³H]-E2 at a concentration of 1000·IC₅₀(E2) were classified as RBA <0.1%. The results obtained with the title compounds I-78 are shown in tables 1 to 3.

TABLE 1
Inhibition of human 17β-HSD2 and 17β-HSD1
by compounds 1-78 on cell-free protein.

| | IC₅₀ [nM]^{a} | | |
|---|---|---|---|
| compound | 17β-HSD2^{b} | 17β-HSD1^{c} | selectivity factor^{d} |
| 1 | 494 | >100000 | >202 |
| 2 | 594 | 13009 | 22 |
| 3 | 265 | >100000 | >375 |
| 4 | 261 | 64532 | 247 |
| 5 | 11%^{e} | n.i.^{e} | |
| 6 | 638 | 9099 | 14 |
| 7 | 13%^{e} | n.i.^{e} | |
| 8 | 481 | 3801 | 8 |
| 9 | 1169 | 98840 | 85 |
| 10 | 28%^{e} | 9%^{e} | |
| 11 | 517 | >60000 | >115 |
| 12 | 35%^{e} | 13%^{e} | |
| 13 | 40%^{e} | n.i.^{e} | |
| 14 | 18%^{e} | n.i.^{e} | |
| 15 | 36%^{e} | n.i.^{e} | |
| 16 | 28%^{e} | n.i.^{e} | |
| 17 | 31%^{e} | n.i.^{e} | |
| 18 | 37%^{e} | 23%^{e} | |
| 19 | 18%^{e} | 11%^{e} | |
| 20 | 11%^{e} | n.i.^{e} | |
| 21 | n.i.^{e} | n.i.^{e} | |
| 22 | 17%^{e} | 12%^{e} | |
| 23 | 370 | >100000 | >250 |
| 24 | 394 | >5449 | 14 |
| 25 | 496 | >120000 | >240 |
| 26 | 335 | 5105 | 15 |
| 27 | 48%^{e} | n.i.^{e} | |
| 28 | 148 | 2982 | 20 |
| 29 | 49%^{e} | n.i.^{e} | |
| 30 | 185 | 3915 | 21 |
| 31 | n.i.^{e} | n.i.^{e} | |
| 32 | 789 | 43396 | 55 |
| 33 | 221 | >100000 | >450 |
| 34 | 42%^{e} | 10%^{e} | |
| 35 | 370 | >100000 | >265 |
| 36 | 36%^{e} | n.i.^{e} | |
| 37 | 1127 | 55741 | 49 |
| 38 | 520 | >100000 | >190 |
| 39 | 161 | >40000 | >250 |
| 40 | 246 | 18597 | 76 |
| 41 | 326 | 3150 | 10 |
| 42 | 430 | 18040 | 42 |
| 43 | 61 | 4451 | 74 |
| 44 | 40 | 202 | 5 |
| 45 | 278 | 23345 | 84 |
| 46 | 163 | 10645 | 65 |
| 47 | 68 | 7593 | 113 |
| 48 | 33%^{e} | 33%^{e} | |
| 49 | 492 | 14746 | 30 |
| 50 | 410 | 1304 | 3 |
| 51 | 207 | 4337 | 21 |
| 52 | 722 | 1346 | 2 |
| 53 | 48%^{e} | n.i.^{e} | |
| 54 | 27%^{e} | n.i.^{e} | |
| 55 | 147 | 6216 | 42 |
| 56 | 62 | >50.000 | >800 |
| 57 | 313 | 1926 | 6 |
| 58 | 169 | 10573 | 62 |
| 59 | 515 | 9927 | 19 |
| 60 | 49%^{e} | n.i.^{e} | |
| 61 | 242 | >40000 | >165 |
| 62 | 726 | >100000 | >135 |
| 63 | 242 | 5306 | 22 |
| 64 | 58 | 6751 | 116 |
| 65 | 184 | 4812 | 26 |
| 66 | 130 | 5425 | 42 |
| 67 | 450 | 13338 | 30 |
| 68 | 31%^{e} | 12%^{e} | |
| 69 | 48%^{e} | 14%^{e} | |
| 70 | 207 | 11454 | 55 |
| 71 | 644 | 6800 | 11 |
| 72 | 62 | 8209 | 133 |
| 73 | 49%^{e} | 18%^{e} | |
| 74 | 390 | 10221 | 26 |
| 75 | 721 | 12259 | 17 |
| 76 | 137 | 1109 | 8 |
| 77 | n.i.^{e} | n.i.^{e} | |
| 78 | n.i.^{e} | 13%^{e} | |
| 79 | 54%^{e} | 5%^{e} | |
| 80 | 39%^{e} | 15%^{e} | |
| 81 | 75% (288 nM) | 18% (28494 nM) | 99 |
| 82 | 11%^{e} | 18%^{e} | |
| 83 | 61% (664 nM) | 15% (95624 nM) | 144 |
| 84 | 20%^{e} | 0%^{e} | |
| 85 | 47%^{e} | 23%^{e} | |

^{a}Mean value of three determinations, except for 79-82 (two data) and 83 (one data), SD less than 20%;
^{b}Human placenta, microsomal fraction, substrate E2, 500 nM, cofactor NAD⁺, 1500 μM;
^{c}Human placenta, cytosolic fraction, substrate E1, 500 nM, cofactor NADH, 500 μM;
^{d}IC₅₀(17β-HSD2)/IC₅₀(17β-HSD1);
^{e}% inhibition at inhibitor concentration of 1 μM;
n.i. = no inhibition

TABLE 2
Inhibition of 17β-HSD2 in MDA-MB-231 cellular assay.

| | % Inhibition @ 1 μM^{a} |
|---|---|
| compound | MDA-MB-231 cells^{b} |
| 28 | 75% |
| 30 | 60% |
| 33 | 39% |
| 39 | 88% |
| 44 | 99% |
| 47 | 76% |
| 51 | 67% |
| 70 | 50% |
| 56 | 70% |

^{a}MDA-MB-231 cells, substrate [³H]-E2: 200 nM;
^{b}% inhibition tested at 1 μM concentration

TABLE 3
Estrogen receptor (ERaα and ERβ) relative binding affinity

| compound | ERα RBA (%)^{a} | ERβ RBA (%)^{a} |
|---|---|---|
| 1 | <0.1 | <0.1 |
| 2 | <0.1 | <0.1 |
| 3 | <0.1 | <0.1 |
| 4 | <0.1 | <0.1 |
| 6 | <0.1 | <0.1 |
| 11 | <0.1 | <0.1 |
| 23 | <0.1 | <0.1 |
| 24 | <0.1 | <0.1 |
| 25 | <0.1 | <0.1 |
| 26 | <0.1 | <0.1 |
| 28 | <0.1 | <0.1 |
| 30 | <0.1 | <0.1 |
| 33 | <0.1 | <0.1 |
| 35 | <0.1 | <0.1 |
| 39 | <0.1 | <0.1 |
| 40 | <0.1 | <0.1 |
| 42 | <0.1 | <0.1 |
| 43 | <0.1 | <0.1 |
| 45 | <0.1 | <0.1 |
| 46 | <0.1 | <0.1 |
| 47 | <0.1 | <0.1 |
| 51 | <0.1 | <0.1 |
| 55 | <0.1 | <0.1 |
| 56 | <0.1 | <0.1 |
| 58 | <0.1 | <0.1 |
| 61 | <0.1 | <0.1 |
| 63 | <0.1 | <0.1 |
| 64 | <0.1 | <0.1 |
| 65 | <0.1 | <0.1 |
| 66 | <0.1 | <0.1 |
| 67 | <0.1 | <0.1 |
| 70 | <0.1 | <0.1 |
| 71 | <0.1 | <0.1 |
| 72 | <0.1 | <0.1 |
| 74 | <0.1 | <0.1 |
| 75 | <0.1 | <0.1 |

^{a}RBA: Relative Binding Affinity, E2 = 100%

1. A compound having 17beta-hydroxysteroid dehydrogenase type 2 (17β-HSD2) inhibitory activity of the formula (I): 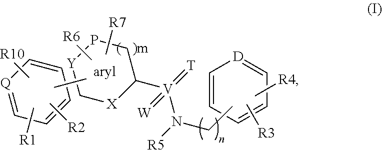
wherein
R1 and R3 are independently selected from H, —OH, alkoxy, acyloxy, alkyl, —N(R8)₂, —SR8, halogen, haloalkyl and phenyl;
R2, R4, R6 and R7 are independently selected from H, —OR8, —COR8, —COOR8, —CONHR8, —OCOR8, —SR8, —SON(R8)₂, —SO₂N(R8)₂, —N(R8)₂, —NHCOR8, —NHSOR8, —NHSO₂R8, -halogen, —NO₂, —CN, —SO₂R8, —CH₂N(R8)₂, —CH₂OR8, aryl optionally substituted with up to three R8, and alkyl optionally substituted with up to three R8;
R5 is lower alkyl;
R8 is selected from H, alkyl optionally substituted with up to three R9, —OH, —NO₂, lower alkoxy, lower alkyl, halogen, haloalkyl, —NH₂, —CN, —NHCOR9, —CONHR9, —NHSO₂R9, —SO₂NHR9, a 5- or 6-membered aromatic or heteroaromatic aryl ring optionally substituted with up to three R9, a homoaromatic ring that is optionally condensed with a 5- or 6-membered, aliphatic or aromatic heterocyclic ring and that (condensed) homoaromatic ring is optionally substituted with up to three R9;
R9 is selected from —OH, alkoxy, halogen, haloalkyl, lower alkyl, —NH₂, —NO₂, —CN and phenyl;
R10 is selected from H, halogen, haloalkyl, lower alkyl;
m is an integer selected from 0 and 1; n is an integer selected from 0 to 2;
the aryl ring is a 5- or 6-membered heteroaromatic ring which carries up to 3 heteroatoms X, Y and P independently selected from N, S, O and said substituents R6 or R7 may be bound to heteroatoms or to ring carbon atoms;
X, Y and P are independently selected from CH, N, N(H), S and O;
Q and D are independently selected from CH and N;
V is C or S; W is O or S; and
T, if V is S, represents O or is absent, or, if V is C, is absent;
or a salt thereof.

2. The compound of claim 1, which has the formula (II): 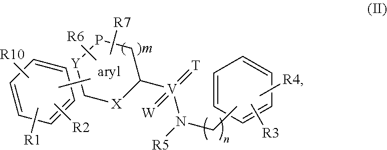
or a salt thereof,
wherein the variables R1, R2, R3, R4, R5, R6, R7, R10, the aryl ring, n, m, X, Y, P, V, W and T, if present, have the same meaning as in claim 1, or a salt thereof.

3. The compound of claim 1, which has the formula (IV): 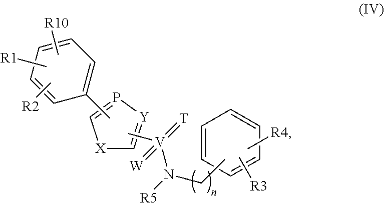
wherein the variables R1, R2, R3, R4, R5, R10, n, X, Y, P, V, W and T, if present, have the same meaning as in claim 1, or a salt thereof.

4. The compound of claim 1, which has the formula (Va) or (Vb): 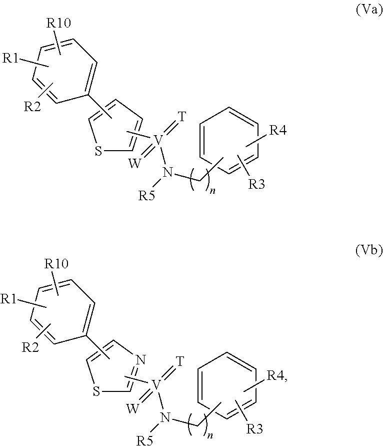
or a salt thereof,
wherein the variables R1, R2, R3, R4, R5, R10, n, V, W and T, if present, have the same meaning as in claim 1, or a salt thereof.

5. The compound according to claim 1, wherein the variables, if present, have the following meaning:
R1 and R3 are independently selected from H, —OH, alkoxy, alkyl, —N(R8)₂, halogen, haloalkyl and phenyl;
R2, R4, R6 and R7 are independently selected from H, —OR8, —COR8, —COOR8, —CONHR8, —OCOR8, —SO₂N(R8)₂, —N(R8)₂, —NHCOR8, —NHSO₂R8, halogen, —CH₂N(R8)₂, —CH₂OR8, aryl optionally substituted with up to three R8, C₁₋₆ alkyl optionally substituted with up to three R8 and halo C₁₋₆ alkyl;
R5 is lower alkyl;
R8 is selected from H, C₁₋₆ alkyl optionally substituted with up to three R9, halogen, halo C₁₋₆ alkyl, —OH, lower alkoxy, —NH₂, and 5- or 6-membered aromatic or heteroaromatic ring optionally substituted with up to three R9;
R9 is selected from —OH, alkoxy, halogen, haloalkyl, C₁₋₄ alkyl, —NH₂ and phenyl;
X, Y and P are independently selected from CH, N, N(H) and S; and/or
n is 0 or 1.

6. The compound of claim 1, which has the formula (VIIa) or (VIIb); 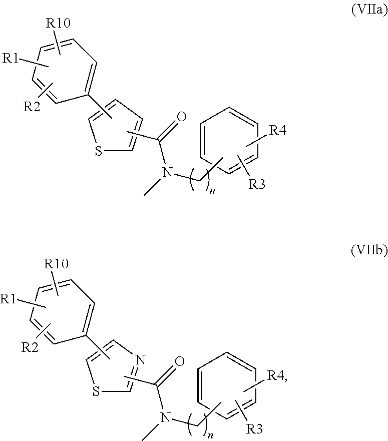
wherein the variables R1 and R3 are independently selected from H, —OH, —OMe, -Me, —NMe₂ and phenyl; R2, R4 and R10 are independently selected from H and halogen; and n is 0 or 1; or a salt thereof.

7. The compound of claim 1, which has the formula (VIIIa), (VIIIb) or (VIIIc): 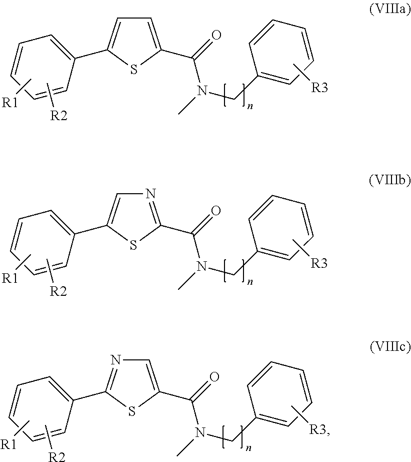
wherein R1 and R3 are independently selected from —OH, —OCH₃ and —CH₃; R2 is H, fluoro or methyl; and n is 0 or 1; or a salt thereof.

8. The compound according to claim 1, which is selected from the group consisting of
3′-methoxy-N-(3-methoxybenzyl)-N-methylbiphenyl-4-carboxamide (1),
3′-hydroxy-N-(3-hydroxybenzyl)-N-methylbiphenyl-4-carboxamide (2),
N-(3-methoxybenzyl)-N,3′-dimethylbiphenyl-4-carboxamide (3),
N-(3-hydroxybenzyl)-N,3′-dimethylbiphenyl-4-carboxamide (4),
4′-methoxy-N-(3-methoxybenzyl)-N-methylbiphenyl-3-carboxamide (5),
4′-hydroxy-N-(3-hydroxybenzyl)-N-methylbiphenyl-3-carboxamide (6),
3′-methoxy-N-(3-methoxybenzyl)-N-methylbiphenyl-3-carboxamide (7,
3′-hydroxy-N-(3-hydroxybenzyl)-N-methylbiphenyl-3-carboxamide (8),
4′-methoxy-N-(3-methoxyphenyl)-N-methylbiphenyl-3-carboxamide (9),
4′-hydroxy-N-(3-hydroxyphenyl)-N-methylbiphenyl-3-carboxamide (10),
3′-methoxy-N-(3-methoxyphenyl)-N-methylbiphenyl-3-carboxamide (11),
3′-hydroxy-N-(3-hydroxyphenyl)-N-methylbiphenyl-3-carboxamide (12),
2′-methoxy-N-(3-methoxyphenyl)-N-methylbiphenyl-3-carboxamide (13),
2′-hydroxy-N-(3-hydroxyphenyl)-N-methylbiphenyl-3-carboxamide (14),
N-(3-methoxyphenyl)-N-methylbiphenyl-3-carboxamide (15),
N-(3-hydroxyphenyl)-N-methylbiphenyl-3-carboxamide (16),
N-(3-methoxyphenethyl)-5-(3-methoxyphenyl)-N-methylthiophene-2-carboxamide (17),
N-(3-hydroxyphenethyl)-5-(3-hydroxyphenyl)-N-methylthiophene-2-carboxamide (18),
N-(3-methoxyphenethyl)-N-methyl-5-phenylthiophene-2-carboxamide (19),
N-(3-hydroxyphenethyl)-N-methyl-5-phenylthiophene-2-carboxamide (20),
5-(3-fluorophenyl)-N-(3-methoxyphenethyl)-N-methylthiophene-2-carboxamide (21),
5-(3-fluorophenyl)-N-(3-hydroxyphenethyl)-N-methylthiophene-2-carboxamide (22),
N-(3-methoxybenzyl)-5-(3-methoxyphenyl)-N-methylthiophene-2-carboxamide (23),
N-(3-hydroxybenzyl)-5-(3-hydroxyphenyl)-N-methylthiophene-2-carboxamide (24),
N-(3-methoxybenzyl)-5-(4-methoxyphenyl)-N-methylthiophene-2-carboxamide (25),
N-(3-hydroxybenzyl)-5-(4-hydroxyphenyl)-N-methylthiophene-2-carboxamide (26),
N-(3-methoxybenzyl)-5-(2-methoxyphenyl)-N-methylthiophene-2-carboxamide (27),
N-(3-hydroxybenzyl)-5-(2-hydroxyphenyl)-N-methylthiophene-2-carboxamide (28),
N-(3-methoxybenzyl)-N-methyl-5-phenylthiophene-2-carboxamide (29),
N-(3-hydroxybenzyl)-N-methyl-5-phenylthiophene-2-carboxamide (30),
5-(4-cyanophenyl)-N-(3-methoxybenzyl)-N-methylthiophene-2-carboxamide (31),
5-(4-cyanophenyl)-N-(3-hydroxybenzyl)-N-methylthiophene-2-carboxamide (32),
N-benzyl-5-(3-methoxyphenyl)-N-methylthiophene-2-carboxamide (33),
N-benzyl-5-(3-hydroxyphenyl)-N-methylthiophene-2-carboxamide (34),
N-benzyl-5-(4-methoxyphenyl)-N-methylthiophene-2-carboxamide (35),
N-benzyl-5-(4-hydroxyphenyl)-N-methylthiophene-2-carboxamide (36),
N-benzyl-5-(2-methoxyphenyl)-N-methylthiophene-2-carboxamide (37),
N-benzyl-5-(2-hydroxyphenyl)-N-methylthiophene-2-carboxamide (38),
N-(3-hydroxybenzyl)-5-(4-methoxyphenyl)-N-methylthiophene-2-carboxamide (39),
5-(3-(dimethylamino)phenyl)-N-(3-hydroxybenzyl)-N-methylthiophene-2-carboxamide (40),
5-(3-fluorophenyl)-N-(3-hydroxybenzyl)-N-methylthiophene-2-carboxamide (41),
5-(4-fluorophenyl)-N-(3-hydroxybenzyl)-N-methylthiophene-2-carboxamide (42),
5-(2-fluoro-3-methoxyphenyl)-N-(3-hydroxybenzyl)-N-methylthiophene-2-carboxamide (43),
5-(2-fluoro-3-hydroxyphenyl)-N-(3-hydroxybenzyl)-N-methylthiophene-2-carboxamide (44),
N-(3-hydroxybenzyl)-5-(3-methoxyphenyl)-N-methylthiophene-2-carboxamide (45),
N-(3-hydroxybenzyl)-N-methyl-5-m-tolylthiophene-2-carboxamide (46),
N,5-bis(3-methoxyphenyl)-N-methylthiophene-2-carboxamide (47),
N,5-bis(3-hydroxyphenyl)-N-methylthiophene-2-carboxamide (48),
5-(2-methoxyphenyl)-N-(3-methoxyphenyl)-N-methylthiophene-2-carboxamide (49),
5-(2-hydroxyphenyl)-N-(3-hydroxyphenyl)-N-methylthiophene-2-carboxamide (50),
N-(3-methoxyphenyl)-N-methyl-5-phenylthiophene-2-carboxamide (51),
N-(3-hydroxyphenyl)-N-methyl-5-phenylthiophene-2-carboxamide (52),
5-(4-cyanophenyl)-N-(3-methoxyphenyl)-N-methylthiophene-2-carboxamide (53),
5-(4-cyanophenyl)-N-(3-hydroxyphenyl)-N-methylthiophene-2-carboxamide (54),
N-(3-methoxyphenyl)-5-(4-methoxyphenyl)-N-methylthiophene-2-carboxamide (55),
5-(2-fluoro-3-methoxyphenyl)-N-(3-methoxyphenyl)-N-methylthiophene-2-carboxamide (56),
5-(2,4-dimethoxyphenyl)-N-(3-methoxyphenyl)-N-methylthiophene-2-carboxamide (57),
5-(3-(dimethylamino)phenyl)-N-(3-methoxyphenyl)-N-methylthiophene-2-carboxamide (58),
5-(3-fluorophenyl)-N-(3-methoxyphenyl)-N-methylthiophene-2-carboxamide (59),
5-(3,4-difluorophenyl)-N-(3-methoxyphenyl)-N-methylthiophene-2-carboxamide (60),
5-(3-fluoro-4-methoxyphenyl)-N-(3-methoxyphenyl)-N-methylthiophene-2-carboxamide (61),
5-(4-fluoro-3-methoxyphenyl)-N-(3-methoxyphenyl)-N-methylthiophene-2-carboxamide (62),
N-(3-methoxyphenyl)-N-methyl-5-(3-(methylthio)phenyl)thiophene-2-carboxamide (63),
N-(3-methoxyphenyl)-N-methyl-5-m-tolylthiophene-2-carboxamide (64),
5-(2,6-difluoro-3-methoxyphenyl)-N-(3-methoxyphenyl)-N-methylthiophene-2-carboxamide (65),
5-(2-fluoro-3-methylphenyl)-N-(3-methoxyphenyl)-N-methylthiophene-2-carboxamide (66),
5-(3-methoxyphenyl)-N-methyl-N-phenylthiophene-2-carboxamide (67),
5-(3-hydroxyphenyl)-N-methyl-N-phenylthiophene-2-carboxamide (68),
N-methyl-N,5-diphenylthiophene-2-carboxamide (69),
5-(3-methoxyphenyl)-N-methyl-N-m-tolylthiophene-2-carboxamide (70),
5-(3-hydroxyphenyl)-N-methyl-N-m-tolylthiophene-2-carboxamide (71),
5-(2-fluoro-3-methoxyphenyl)-N-methyl-N-(m-tolylthiophene)-2-carboxamide (72),
5-(2-fluoro-3-methoxyphenyl)-N-(4-methoxyphenyl)-N-methylthiophene-2-carboxamide (73),
N-(2-fluorophenyl)-N-methyl-5-m-tolylthiophene-2-carboxamide (74),
5-(3-methoxyphenyl)-N-methyl-N-(3-(trifluoromethyl)phenyl)thiophene-2-carboxamide (75),
N-(biphenyl-3-yl)-5-(3-methoxyphenyl)-N-methylthiophene-2-carboxamide (76),
5-(2-fluoro-3-methoxyphenyl)-N-(3-methoxyphenyl)thiophene-2-carboxamide (77),
5-(2-fluoro-3-methoxyphenyl)-N-(3-methoxyphenyl)-N-phenylthiophene-2-carboxamide (78),
N-(3-methoxyphenyl)-N-methyl-2-(3-methylphenyl)-1,3-thiazole-5-carboxamide (79),
2-(2-fluoro-3-methoxyphenyl)-N-(3-hydroxybenzyl)-N-methyl-1,3-thiazole-5-carboxamide (80),
N-(3-methoxyphenyl)-N-methyl-5-(3-methylphenyl)-1,3-thiazole-2-carboxamide (81),
N-cyclopropyl-N-(3-methoxyphenyl)-5-(3-methylphenyl)thiophene-2-carboxamide (82),
N-cyclopropyl-5-(2-fluoro-3-methoxyphenyl)-N-(3-hydroxybenzyl)thiophene-2-carboxamide (83),
N-(3-methoxyphenyl)-N-methyl-5-(3-methylphenyl)thiophene-2-sulfonamide (84) and
5-(2-fluoro-3-methoxyphenyl)-N-(3-hydroxybenzyl)-N-methylthiophene-2-sulfonamide (85),
or a salt thereof.

9. A process for producing a compound formula (I) or a salt thereof according to claim, which comprises condensing an activated acyl or sulfonyl compound of the formula (B) with a secondary amine of the formula (C), followed by a cross-coupling reaction with a compound of the formula (A): 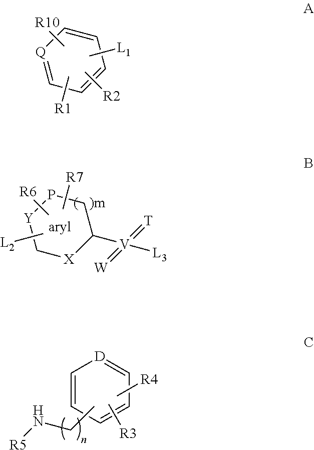
wherein all the variables are as defined in claim 1, and L1, L2 and L3 are leaving groups.

10. A method for the treatment and/or prophylaxis of a sex steroid-deficient disease in a patient in need thereof, said method comprising administering to said patient an effective amount therefor of a compound having 17β-HSD2 inhibitory activity according to claim 1 or a pharmaceutically acceptable salt thereof.

11. The method according to claim 10, wherein the sex steroid-deficient disease is selected from the group consisting of diseases caused by a misbalance of OB/OC activity, osteoporosis, osteopenia, bone repair, post-menopausal symptoms, vaginal atrophy, colon cancer, neuronal diseases, Alzheimer's disease, Parkinson's disease, depression, anxiety, hypercholesterolemia, cardiovascular diseases, hair loss, rheumatic diseases and inflammatory diseases.

12. A pharmaceutical composition comprising at least one compound having 17β-HSD2 inhibitory activity according to claim 1 or a pharmaceutically acceptable salt thereof, and optionally a suitable carrier or excipient.

13. The pharmaceutical composition of claim 12, which is formulated for the treatment and prophylaxis of a sex steroid deficient disease selected from the group consisting of diseases caused by a misbalance of OB/OC activity, osteoporosis, osteopenia, bone repair, post-menopausal symptoms, vaginal atrophy, colon cancer, neuronal diseases, Alzheimer's disease, Parkinson's disease, depression, anxiety, hypercholesterolemia, cardiovascular diseases, hair loss, rheumatic diseases and inflammatory diseases.

14. (canceled)

15. A method for the treatment and prophylaxis of osteoporosis in a patient in need thereof, which method comprises administering the patient an effective amount therefor of a compound having 17β-HSD2 inhibitory activity according to claim 1 or a pharmaceutically acceptable salt thereof.
